(19) United States
(12) Patent Application Publication
(10) Pub. No.: US 2022/0170044 A1
(43) Pub. Date: Jun. 2, 2022

(54) TRANSPOSON-BASED MODIFICATIONS OF IMMUNE CELLS

(71) Applicants: DNA Twopointo, Inc., Newark, CA (US); The General Hospital Corporation, Boston, MA (US)

(72) Inventors: Mark Cobbold, Winchester, MA (US); Maggie Lee, San Jose, CA (US); Jeremy Minshull, Los Altos, CA (US); Feng Shi, Winchester, MA (US); Yifang Shui, Boston, MA (US)

(73) Assignees: DNA Twopointo, Inc., Newark, CA (US); The General Hospital Corporation, Boston, MA (US)

(21) Appl. No.: 17/429,342

(22) PCT Filed: Feb. 7, 2020
(86) PCT No.: PCT/US20/17283

§ 371 Date: Aug. 8, 2021

Related U.S. Application Data
(60) Provisional application No. 62/803,142, filed on Feb. 8, 2019.

(51) Int. Cl. C12N 15/86; C07K 14/47; A61K 35/17; C07K 14/705; C07K 14/725

(52) U.S. Cl. CPC C12N 15/86; C07K 14/4705; A61K 35/17; C07K 14/4747; C12N 2740/16043; C07K 14/7051; C07K 14/70578; C12N 2800/90; C07K 14/70521

(57) ABSTRACT

The present invention provides methods and compositions for stable genetic modification of immune cells. The genetic modifications can be used to produce immune cells for therapeutic or diagnostic purposes.

### CROSS REFERENCE TO RELATED APPLICATIONS

The present application claims priority from 62/803,142 filed Feb. 8, 2019, incorporated by reference in its entirety for all purposes.

### REFERENCE TO A SEQUENCE LISTING

The application refers to sequences disclosed in a txt file named ST25_20200128, of 889,000 bytes, created Jan. 28, 2020, incorporated by reference.

### BACKGROUND OF THE INVENTION

Genetic modification of immune cells can be used to modify their properties. Genetically modifiable immune cell properties include the molecules that are recognized by the immune cell, cellular responses within the immune cell, the ability of the immune cell to survive under certain environmental conditions, and the proteins produced by the immune cell. Genetic modifications of immune cells can be used to improve their disease-targeting response. By enhancing the function of specific immune cells, the immune response may be augmented, for example to achieve long-lasting cancer regression.

Stable genetic modifications of immune cells can be made by integrating a heterologous polynucleotide into the genome of the immune cell. Heterologous DNA may be introduced into immune cells in different ways: by transfecting with naked plasmid DNA, by packaging the DNA into viral particles used to infect the immune cells, or by introducing into immune cells a transposon and its cognate transposase.

Non-viral vector systems, including plasmid DNA, generally suffer from inefficient cellular delivery, pronounced cellular toxicity and limited duration of transgene expression due to the lack of genomic insertion and resulting degradation and/or dilution of the vector in transfected cell populations. Transgenes delivered by non-viral approaches often form long, repeated arrays (concatemers) that are targets for transcriptional silencing by heterochromatin formation.

Viral packaging generally imposes limits on the size of the DNA that can be inserted into the viral vector. Some viruses (such as AAV) are retained as non-replicated episomes that are therefore diluted out as cells divide. For viruses that integrate their genomes into the target cell genome there are safety concerns regarding viral integration sites. For all viral delivery methods there are concerns about the costs of viral manufacture and potential immunogenicity of viral components.

Transposons provide an alternative delivery system that is as simple to manufacture and non-immunogenic as naked DNA, but highly efficient at integrating into the target cell genome. Transposons comprise two ends that are recognized by a transposase. The transposase acts on the transposon to excise it from one DNA molecule and integrate it into another: this process is referred to as transposition. The DNA between the two transposon ends is transposed by the transposase along with the transposon ends. Heterologous DNA flanked by a pair of transposon ends, such that it is recognized and transposed by a transposase is referred to herein as a synthetic transposon. Introduction of a synthetic transposon and a corresponding transposase into the nucleus of a eukaryotic cell may result in transposition of the transposon into the genome of the cell. Transposon/transposase gene delivery platforms have the potential to overcome the limitations of naked DNA and viral delivery. In particular, the piggyBac-like transposons are attractive because of their unlimited gene cargo capacity.

The expression levels of genes encoded on a polynucleotide integrated into the genome of a cell depend on the configuration of sequence elements within the polynucleotide. The efficiency of integration and thus the number of copies of the polynucleotide that are integrated into each genome, and the genomic loci where integration occurs also influence the expression levels of genes encoded on the polynucleotide. The efficiency with which a polynucleotide may be integrated into the genome of a target cell can often be increased by placing the polynucleotide into a transposon.

Transposition by a piggyBac-like transposase is perfectly reversible. The transposon is integrated at an integration target sequence in a recipient DNA molecule, during which the target sequence becomes duplicated at each end of the transposon inverted terminal repeats (ITRs). Subsequent transposition removes the transposon and restores the recipient DNA to its former sequence, with the target sequence duplication and the transposon removed. However, this is not sufficient to remove a transposon from a genome into which it has been integrated, as it is highly likely that the transposon will be excised from the first integration target sequence but integrated into a second integration target sequence in the genome. Transposases that are deficient for the integration function, on the other hand, can excise the transposon from the first target sequence, but will be unable to integrate into a second target sequence. Integration-deficient transposases are thus useful for reversing the genomic integration of a transposon.

### SUMMARY OF THE INVENTION

Transposons capable of stably modifying the genome of immune cells are an aspect of the invention. Genes that are advantageous in modifying immune cells to enhance their function are an aspect of the invention. Methods for modifying immune cells to enhance their function are an aspect of the invention.

Methods for modifying the genomes of immune cells are described. Immune cells include lymphocytes such as T-cells and B-cells and natural killer cells, T-helper cells, antigen-presenting cells, dendritic cells, neutrophils and macrophages. Modifications include enhancing the ability of an immune cell to survive and/or proliferate under certain conditions or in certain environments, altering the amount or type of proteins expressed on the immune cell surface, and altering the response of the immune cell to proteins or small molecules that contact the cell. Sequences of polynucleotide constructs for effecting genomic modifications of immune cells are provided and are an aspect of the invention.

The ability to enhance the function, persistence and proliferation of human T-cells is a current bottle neck for cancer immunotherapy. Technologies that allow improved performance, expansion and genetic manipulation of T-cells are in high demand. The ability to control and expand T-cells has many applications, including the following. (i) Improving the function of T-cell therapy for greater efficacy and or safety, for example in combination with CAR-T. (ii) Reversing T-cell exhaustion and/or restimulation-induced cell death of tumor infiltrating T-cells, allowing T-cells to survive and function within the tumor microenvironment. (iii) Improving the survival of human T-cells in mice for preclinical study (in vivo). (iv) Identification of antigen-specific T-cells and cloning T-cell receptors in vitro. (v) Developing T-cell lines that can be maintained ex-vivo, and that still perform biological functions of T-cells (such as cell killing).

Immune cell survival-enhancing genes include anti-apoptotic genes such as Survivin, Bcl2, Bcl6, Bcl-XL and genes encoding mutants of the normal apoptotic pathway that exert a dominant negative effect such as dominant negative mutants of Casp3, Casp7, Casp8, Casp9 or Casp10. Immune cell survival-enhancing genes also include activating mutants of STAT3, including STAT3 mutants comprising one of the following mutations: F174S, H410R, S614R, E616K, G618R, Y640F, N6471, E652K, K658Y, K658R, K658N, K658M, K658R, K658H, K658N, D661Y or D661V. Immune cell survival-enhancing genes also include activating mutants of CD28, including CD28 mutants comprising one of the following mutations: D124E, D124V, T1951 or T195P. Immune cell survival-enhancing genes also include activating mutants of RhoA, including RhoA mutants comprising one of the following mutations: G17V or K18N. Immune cell survival-enhancing genes also include activating mutants of phospholipase C gamma, including phospholipase C gamma mutants comprising one of the following mutations: S345F, S520F or R707Q. Immune cell survival-enhancing genes also include activating mutants of STAT5B, including STAT5B mutants comprising one of the following mutations: N642H, T648S, S652Y, Y665F or P267A. Immune cell survival-enhancing genes also include activating mutants of CCND1, including CCND1 mutants comprising one of the following mutations: E36G, E36Q, E36K, A39S, S41L, S41P, S41T, V42E, V42A, V42L, V42M, Y44S, Y44D, Y44C, Y44H, K46T, K46R, K46N, K46E, C47G, C47R, C47S, C47W, P199R, P199S, P199L, S201F, T2851, T285A, P286L, P286H, P286S, P286T or P286A.

Immune cell survival-enhancing genes also include enhanced signaling receptor (ESR) wherein the ESR comprises a sequence derived from the extracellular domain of a receptor that normally transmits an inhibitory signal to an immune cell, a sequence derived from the intracellular domain of an intracellular domain of a receptor that transmits a stimulatory signal to an immune cell and a transmembrane domain. Exemplary extracellular domains include a human protein selected from TNFRSF3 (LTRβ), TNFRSF6 (Fas), TNFRSF8 (CD30), TNFRSF10A (DR4), TNFRSF10B (DR5), TNFRSF19 (TROY), TNFRSF21 (DR6) and CTLA4, such as SEQ ID NOs: 322-340. Exemplary intracellular domains include a human protein selected from TNFRSF4 (OX40), TNFRSF5 (CD40), TNFRSF7 (CD27), TNFRSF9 (4-1BB), TNFRSF11A (RANK), TNFRSF13B (TACI), TNFRSF13C (BAFF-R), TNFRSF14 (HVEM), TNFRSF17 (CD269), TNFRSF18 (GITR), CD28, CD28H (TMIGD2), Inducible T-cell Costimulator (ICOS/CD278), DNAX Accessory Molecule-1 (DNAM-1/CD226), Signaling Lymphocytic Activation Molecule (SLAM/CD150), T-cell Immunoglobulin and Mucin domain (TIM-1/HAVcr-1), interferon receptor alpha chain (IFNAR1), interferon receptor beta chain IFNAR2), interleukin-2 receptor beta subunit (IL2RB), interleukin-2 receptor gamma subunit (IL2RG), Tumor Necrosis Factor Superfamily 14 (TNFSF14/LIGHT), Natural Killer Group 2 member D (NKG2D/CD314) and CD40 ligand (CD40L), such as SEQ ID NOs: 341-364. Exemplary transmembrane domains include a human protein selected from 365-396. Exemplary enhanced signaling receptors include sequences comprising a sequence selected from SEQ ID NOs: 274-318.

Preferably immune cell survival-enhancing genes are provided to immune cells using transposon vectors. Transposons are efficiently integrated into an immune cell genome by a corresponding transposase. Several different classes of transposons are useful for integrating genes into the genome of an immune cell. PiggyBac-like transposons such as the looper moth piggyBac transposon which comprises ITR sequences comprising SEQ ID NO: 18 and 19, or the piggyBat transposon which comprises ITR sequences comprising SEQ ID NO: 20 and 21, or the Xenopus piggyBac-like transposon which comprises ITR sequences comprising SEQ ID NO: 6 and 7, or the Bombyx piggyBac-like transposon which comprises ITR sequences comprising SEQ ID NO: 14 and 15 can all be transposed by a corresponding transposase into an immune cell genome. Also, Mariner type transposons such as Sleeping Beauty which comprises ITRs comprising SEQ ID NO: 26 and 27 can be transposed by a corresponding transposase into an immune cell genome. Also hAT type transposons such as TcBuster which comprises ITRs comprising SEQ ID NO: 399 and 400 can be transposed by a corresponding transposase into an immune cell genome. Any of these transposons may be used to integrate survival-enhancing genes into an immune cell genome. A transposon may be introduced into an immune cell with a corresponding transposase. The transposase may be provided as protein, or as a nucleic acid encoding the transposase such as an mRNA molecule or a DNA molecule with a sequence encoding the transposase operably linked to a promoter expressible in the immune cell. Other genes may also be introduced into the immune cell to modify its function. For example, genes encoding receptors that allow the immune cell to bind to antigens on the surface of a target cell may be introduced. Genes that can be used to kill the immune cell may also be introduced. The benefit of using a transposon to deliver combinations of genes to the immune cell is that a transposase typically integrates all of the DNA between the transposon ITRs into the genome of the immune cell. Thus multiple genes can be introduced simultaneously.

It is feasible to integrate immune cell survival genes into the genome of an immune cell precursor such as a stem cell, and then differentiate the immune cell precursor into an immune cell. Such manipulations are expressly contemplated. To enhance survival of the immune cell, a survival-enhancing gene should be operably linked to a promoter such that the survival-enhancing gene is expressible in the immune cell. Exemplary promoters include an EF1 promoter, a PGK promoter, a GAPDH promoter, an EEF2 promoter, a ubiquitin promoter, an SV40 promoter or an HSVTK promoter, such as a promoter selected from SEQ ID NOs 94-154.

Modified human immune cells are an aspect of the invention. In addition, animal immune cells that have been modified to enhance their survival or proliferation are also of value as experimental models and as animal therapeutic agents. Modified immune cells from mammals including primates, rodents, cats, dogs and horses are an aspect of the invention.

### BRIEF DESCRIPTION OF THE FIGURES

FIG. 1. FACS analysis of Jurkat human T-cell line transfected with Xenopus or Bombyx piggyBac-like gene transfer systems. Human Jurkat cells were transfected with transposases and corresponding transposons comprising a CD19 gene as described in Section 6.1.1. After 70 days, CD19-expressing cells were selected using a FACS sorter and grown in culture for a further 85 days. Cells were then stained for CD19 and analyzed on a FACS. Panel A: Cells originally transfected with transposon with sequence given by SEQ ID NO: 223 were analyzed for CD19 (y-axis) and GFP (x-axis). Panel B: Cells originally transfected with transposon with sequence given by SEQ ID NO: 224 were analyzed for CD19 (y-axis) and RFP (x-axis).

FIG. 2. FACS analysis of primary T-cells transfected with a gene encoding a mutated STAT3. Human primary T-cells were co-transfected with a transposase and a corresponding transposon comprising a gene encoding a mutated version of STAT3: STAT3-Y640F, and a gene encoding a green fluorescent protein (GFP). Panel A: Cells were cultured for various times (indicated at the top), after which samples were taken, labelled with a fluorescently-labelled anti-CD8 antibody and analyzed using a fluorescence-activated cell sorter. CD8 expressed on the surface of T-cells is shown on the y-axis, GFP (which indicates the presence of the transposon comprising the STAT3 Y640F gene) is shown along the x-axis. Panel B: the fraction of CD8+ cells showing GFP fluorescence was calculated from the data shown in Panel A.

FIG. 3. FACS analysis of a mixture of transfected primary T-cells and cells from a JY B-cell line. Human primary T-cells were co-transfected with a transposase and one of three corresponding transposons comprising a gene encoding a chimeric antigen receptor with sequence given by SEQ ID NO: 229 and a GFP reporter as described in Section 6.2.1.3. One transposon comprised no further genes (Panels A and D), one transposon further comprised a gene encoding Survivin (Panels B and E) and one transposon further comprised a gene encoding CD28-D124E-T195P (Panels C and F). Cells were cultured for approximately 5 weeks, at which point approximately 10% of the T-cells were expressing GFP. At that point 200,000 T-cells (=20,000 GFP-expressing T-cells) were mixed with 200,000 cells of the JY transformed B-cell line. Three days (Panels A, C and E) or 7 days (Panels B, D and F) post-mixing, cells were labelled with fluorescently-labelled anti-CD8 and anti-CD19 antibodies and analyzed using a fluorescence-activated cell sorter. CD8 expressed on the surface of T-cells is shown on the y-axis, CD19 expressed on the surface of the JY cells is shown on the x-axis.

FIG. 4. FACS analysis of primary T-cells transfected with genes encoding Bcl-2 and Bel-6. Human primary T-cells were co-transfected with a transposase and a corresponding transposon comprising a gene encoding Bcl2 and Bcl6, and a gene encoding a green fluorescent protein (GFP). Panel A: Cells were cultured for various times (indicated at the top), after which samples were taken, labelled with a fluorescently-labelled anti-CD8 antibody and analyzed using a fluorescence-activated cell sorter. CD8 expressed on the surface of T-cells is shown on the y-axis, GFP (which indicates the presence of the transposon comprising the Bcl2-2A-Bcl6 gene) is shown along the x-axis. Panel B: the fraction of CD8+ cells showing GFP fluorescence was calculated from the data shown in Panel A.

FIG. 5. FACS analysis of primary T-cells from 3 donors transfected with a gene encoding Bcl-XL. Human primary T-cells were co-transfected with a transposase and a corresponding transposon comprising a gene encoding Bcl-XL, and a gene encoding a green fluorescent protein (GFP). Cells were grown in culture for 240 days, stained with a fluorescently-labeled antibody against human CD8 and analyzed on a flow cytometer. Panel A: Donor 81; Panel A: Donor 82; Panel A: Donor 84. CD8 expressed on the surface of T-cells is shown on the y-axis, GFP (which indicates the presence of the transposon comprising the Bcl-XL gene) is shown along the x-axis.

### DESCRIPTION

### Definitions

Use of the singular forms “a,” “an,” and “the” include plural references unless the context clearly dictates otherwise. Thus, for example, reference to “a polynucleotide” includes a plurality of polynucleotides, reference to “a substrate” includes a plurality of such substrates, reference to “a variant” includes a plurality of variants, and the like.

Terms such as “connected,” “attached,” “linked,” and “conjugated” are used interchangeably herein and encompass direct as well as indirect connection, attachment, linkage or conjugation unless the context clearly dictates otherwise. Where a range of values is recited, it is to be understood that each intervening integer value, and each fraction thereof, between the recited upper and lower limits of that range is also specifically disclosed, along with each subrange between such values. The upper and lower limits of any range can independently be included in or excluded from the range, and each range where either, neither or both limits are included is also encompassed within the invention. Where a value being discussed has inherent limits, for example where a component can be present at a concentration of from 0 to 100%, or where the pH of an aqueous solution can range from 1 to 14, those inherent limits are specifically disclosed. Where a value is explicitly recited, it is to be understood that values which are about the same quantity or amount as the recited value are also within the scope of the invention. Where a combination is disclosed, each sub combination of the elements of that combination is also specifically disclosed and is within the scope of the invention. Conversely, where different elements or groups of elements are individually disclosed, combinations thereof are also disclosed. Where any element of an invention is disclosed as having a plurality of alternatives, examples of that invention in which each alternative is excluded singly or in any combination with the other alternatives are also hereby disclosed; more than one element of an invention can have such exclusions, and all combinations of elements having such exclusions are hereby disclosed.

Unless defined otherwise herein, all technical and scientific terms used herein have the same meaning as commonly understood by one of ordinary skill in the art to which this invention belongs. Singleton, et al., Dictionary of Microbiology and Molecular Biology, 2nd Ed., John Wiley and Sons, New York (1994), and Hale & Marham, The Harper Collins Dictionary of Biology, Harper Perennial, N Y, 1991, provide one of skill with a general dictionary of many of the terms used in this invention. Although any methods and materials similar or equivalent to those described herein can be used in the practice or testing of the present invention, the preferred methods and materials are described. Unless otherwise indicated, nucleic acids are written left to right in 5′ to 3′ orientation; amino acid sequences are written left to right in amino to carboxy orientation, respectively. The terms defined immediately below are more fully defined by reference to the specification as a whole.

The “configuration” of a polynucleotide means the functional sequence elements within the polynucleotide, and the order and direction of those elements.

The terms “corresponding transposon” and “corresponding transposase” are used to indicate an activity relationship between a transposase and a transposon. A transposase transposases its corresponding transposon.

The term “counter-selectable marker” means a polynucleotide sequence that confers a selective disadvantage on a host cell. Examples of counter-selectable markers include sacB, rpsL, tetAR, pheS, thyA, gata-1, ccdB, kid and barnase (Bernard, 1995, Journal/Gene, 162: 159-160; Bernard et al., 1994. Journal/Gene, 148: 71-74; Gabant et al., 1997, Journal/Biotechniques, 23: 938-941; Gababt et al., 1998, Journal/Gene, 207: 87-92; Gababt et al., 2000, Journal/Biotechniques, 28: 784-788; Galvao and de Lorenzo, 2005, Journal/Appl Environ Microbiol, 71: 883-892; Hartzog et al., 2005, Journal/Yeat, 22:789-798; Knipfer et al., 1997, Journal/Plasmid, 37: 129-140; Reyrat et al., 1998, Journal/Infect Immun, 66: 4011-4017; Soderholm et al., 2001, Journal/Biotechniques, 31: 306-310, 312; Tamura et al., 2005, Journal/Appl Environ Microbiol, 71: 587-590; Yazynin et al., 1999, Journal/FEBS Lett, 452: 351-354). Counter-selectable markers often confer their selective disadvantage in specific contexts. For example, they may confer sensitivity to compounds that can be added to the environment of the host cell, or they may kill a host with one genotype but not kill a host with a different genotype. Conditions which do not confer a selective disadvantage on a cell carrying a counter-selectable marker are described as “permissive”. Conditions which do confer a selective disadvantage on a cell carrying a counter-selectable marker are described as “restrictive”.

The term “coupling element” or “translational coupling element” means a DNA sequence that allows the expression of a first polypeptide to be linked to the expression of a second polypeptide. Internal ribosome entry site elements (IRES elements) and cis-acting hydrolase elements (CHYSEL elements) are examples of coupling elements.

The terms “DNA sequence”, “RNA sequence” or “polynucleotide sequence” mean a contiguous nucleic acid sequence. The sequence can be an oligonucleotide of 2 to 20 nucleotides in length to a full length genomic sequence of thousands or hundreds of thousands of base pairs.

The term “Enhanced Signaling Receptor” (or “ESR”) means a protein in which the extracellular/ligand binding domain of a receptor that transmits an inhibitory signal to an immune cell, is fused to the intracellular domain of a receptor that transmits a stimulatory signal.

The term “expression construct” means any polynucleotide designed to transcribe an RNA. For example, a construct that contains at least one promoter which is or may be operably linked to a downstream gene, coding region, or polynucleotide sequence (for example, a cDNA or genomic DNA fragment that encodes a polypeptide or protein, or an RNA effector molecule, for example, an antisense RNA, triplex-forming RNA, ribozyme, an artificially selected high affinity RNA ligand (aptamer), a double-stranded RNA, for example, an RNA molecule comprising a stem-loop or hairpin dsRNA, or a bi-finger or multi-finger dsRNA or a microRNA, or any RNA). An “expression vector” is a polynucleotide comprising a promoter which can be operably linked to a second polynucleotide. Transfection or transformation of the expression construct into a recipient cell allows the cell to express an RNA effector molecule, polypeptide, or protein encoded by the expression construct. An expression construct may be a genetically engineered plasmid, virus, recombinant virus, or an artificial chromosome derived from, for example, a bacteriophage, adenovirus, adeno-associated virus, retrovirus, lentivirus, poxvirus, or herpesvirus. Such expression vectors can include sequences from bacteria, viruses or phages. Such vectors include chromosomal, episomal and virus-derived vectors, for example, vectors derived from bacterial plasmids, bacteriophages, yeast episomes, yeast chromosomal elements, and viruses, vectors derived from combinations thereof, such as those derived from plasmid and bacteriophage genetic elements, cosmids and phagemids. An expression construct can be replicated in a living cell, or it can be made synthetically. For purposes of this application, the terms “expression construct”, “expression vector”, “vector”, and “plasmid” are used interchangeably to demonstrate the application of the invention in a general, illustrative sense, and are not intended to limit the invention to a particular type of expression construct.

The term “expression polypeptide” means a polypeptide encoded by a gene on an expression construct.

The term “expression system” means any in vivo or in vitro biological system that is used to produce one or more gene product encoded by a polynucleotide.

A “gene transfer system” comprises a vector or gene transfer vector, or a polynucleotide comprising the gene to be transferred which is cloned into a vector (a “gene transfer polynucleotide” or “gene transfer construct”). A gene transfer system may also comprise other features to facilitate the process of gene transfer. For example, a gene transfer system may comprise a vector and a lipid or viral packaging mix for enabling a first polynucleotide to enter a cell, or it may comprise a polynucleotide that includes a transposon and a second polynucleotide sequence encoding a corresponding transposase to enhance productive genomic integration of the transposon. The transposases and transposons of a gene transfer system may be on the same nucleic acid molecule or on different nucleic acid molecules. The transposase of a gene transfer system may be provided as a polynucleotide or as a polypeptide.

Two elements are “heterologous” to one another if not naturally associated. For example, a nucleic acid sequence encoding a protein linked to a heterologous promoter means a promoter other than that which naturally drives expression of the protein. A heterologous nucleic acid flanked by transposon ends or ITRs means a heterologous nucleic acid not naturally flanked by those transposon ends or ITRs, such as a nucleic acid encoding a polypeptide other than a transposase, including an antibody heavy or light chain. A nucleic acid is heterologous to a cell if not naturally found in the cell or if naturally found in the cell but in a different location (e.g., episomal or different genomic location) than the location described.

The term “host” means any prokaryotic or eukaryotic organism that can be a recipient of a nucleic acid. A “host,” as the term is used herein, includes prokaryotic or eukaryotic organisms that can be genetically engineered. For examples of such hosts, see Maniatis et al., Molecular Cloning. A Laboratory Manual, Cold Spring Harbor Laboratory, Cold Spring Harbor, N.Y. (1982). As used herein, the terms “host,” “host cell,” “host system” and “expression host” can be used interchangeably.

An “IRES” or “internal ribosome entry site” means a specialized sequence that directly promotes ribosome binding, independent of a cap structure.

An ‘isolated’ polypeptide or polynucleotide means a polypeptide or polynucleotide that has been either removed from its natural environment, produced using recombinant techniques, or chemically or enzymatically synthesized. Polypeptides or polynucleotides of this invention may be purified, that is, essentially free from any other polypeptide or polynucleotide and associated cellular products or other impurities.

The terms “nucleoside” and “nucleotide” include those moieties which contain not only the known purine and pyrimidine bases, but also other heterocyclic bases which have been modified. Such modifications include methylated purines or pyrimidines, acylated purines or pyrimidines, or other heterocycles. Modified nucleosides or nucleotides can also include modifications on the sugar moiety, for example, where one or more of the hydroxyl groups are replaced with halogen, aliphatic groups, or is functionalized as ethers, amines, or the like. The term “nucleotidic unit” is intended to encompass nucleosides and nucleotides.

An “Open Reading Frame” or “ORF” means a portion of a polynucleotide that, when translated into amino acids, contains no stop codons. The genetic code reads DNA sequences in groups of three base pairs, which means that a double-stranded DNA molecule can read in any of six possible reading frames-three in the forward direction and three in the reverse. An ORF typically also includes an initiation codon at which translation may start.

The term “operably linked” refers to functional linkage between two sequences such that one sequence modifies the behavior of the other. For example, a first polynucleotide comprising a nucleic acid expression control sequence (such as a promoter, IRES sequence, enhancer or array of transcription factor binding sites) and a second polynucleotide are operably linked if the first polynucleotide affects transcription and/or translation of the second polynucleotide. Similarly, a first amino acid sequence comprising a secretion signal, or a subcellular localization signal and a second amino acid sequence are operably linked if the first amino acid sequence causes the second amino acid sequence to be secreted or localized to a subcellular location.

The term “overhang” or “DNA overhang” means the single-stranded portion at the end of a double-stranded DNA molecule. Complementary overhangs are those which will base-pair with each other.

A “piggyBac-like transposase” means a transposase with at least 20% sequence identity as identified using the TBLASTN algorithm to the piggyBac transposase from Trichoplusia ni (SEQ ID NO: 30), and as more fully described in Sakar, A. et. al., (2003). Mol. Gen. Genomics 270: 173-180. “Molecular evolutionary analysis of the widespread piggyBac transposon family and related ‘domesticated’ species”, and further characterized by a DDE-like DDD motif, with aspartate residues at positions corresponding to D268, D346, and D447 of Trichoplusia ni piggyBac transposase on maximal alignment. PiggyBac-like transposases are also characterized by their ability to excise their transposons precisely with a high frequency. A “piggyBac-like transposon” means a transposon having transposon ends which are the same or at least 80% and preferably at least 90, 95, 96, 97, 98 or 99% identical to the transposon ends of a naturally occurring transposon that encodes a piggyBac-like transposase. A piggyBac-like transposon includes an inverted terminal repeat (ITR) sequence of approximately 12-16 bases at each end. These repeats may be identical at the two ends, or the repeats at the two ends may differ at 1 or 2 or 3 or 4 positions in the two ITRs. The transposon is flanked on each side by a 4 base sequence corresponding to the integration target sequence which is duplicated on transposon integration (the Target Site Duplication or Target Sequence Duplication or TSD).

The terms “polynucleotide,” “oligonucleotide,” “nucleic acid” and “nucleic acid molecule” and “gene” are used interchangeably to refer to a polymeric form of nucleotides of any length, and may comprise ribonucleotides, deoxyribonucleotides, analogs thereof, or mixtures thereof. This term refers only to the primary structure of the molecule. Thus, the term includes triple-, double- and single-stranded deoxyribonucleic acid (“DNA”), as well as triple-, double- and single-stranded ribonucleic acid (“RNA”). It also includes modified, for example by alkylation, and/or by capping, and unmodified forms of the polynucleotide. More particularly, the terms “polynucleotide,” “oligonucleotide,” “nucleic acid” and “nucleic acid molecule” include polydeoxyribonucleotides (containing 2-deoxy-D-ribose), polyribonucleotides (containing D-ribose), including tRNA, rRNA, hRNA, siRNA and mRNA, whether spliced or unspliced, any other type of polynucleotide which is an N- or C-glycoside of a purine or pyrimidine base, and other polymers containing nonnucleotidic backbones, for example, polyamide (for example, peptide nucleic acids (“PNAs”)) and polymorpholino (commercially available from the Anti-Virals, Inc., Corvallis, Oreg., as Neugene) polymers, and other synthetic sequence-specific nucleic acid polymers providing that the polymers contain nucleobases in a configuration which allows for base pairing and base stacking, such as is found in DNA and RNA. There is no intended distinction in length between the terms “polynucleotide,” “oligonucleotide,” “nucleic acid” and “nucleic acid molecule,” and these terms are used interchangeably herein. These terms refer only to the primary structure of the molecule. Thus, these terms include, for example, 3′-deoxy-2′, 5′-DNA, oligodeoxyribonucleotide N3′ P5′ phosphoramidates, 2′-O-alkyl-substituted RNA, double- and single-stranded DNA, as well as double- and single-stranded RNA, and hybrids thereof including for example hybrids between DNA and RNA or between PNAs and DNA or RNA, and also include known types of modifications, for example, labels, alkylation, “caps,” substitution of one or more of the nucleotides with an analog, internucleotide modifications such as, for example, those with uncharged linkages (for example, methyl phosphonates, phosphotriesters, phosphoramidates, carbamates, or the like) with negatively charged linkages (for example, phosphorothioates, phosphorodithioates, or the like), and with positively charged linkages (for example, aminoalkylphosphoramidates, aminoalkylphosphotriesters), those containing pendant moieties, such as, for example, proteins (including enzymes (for example, nucleases), toxins, antibodies, signal peptides, poly-L-lysine, or the like), those with intercalators (for example, acridine, psoralen, or the like), those containing chelates (of, for example, metals, radioactive metals, boron, oxidative metals, or the like), those containing alkylators, those with modified linkages (for example, alpha anomeric nucleic acids, or the like), as well as unmodified forms of the polynucleotide or oligonucleotide.

A “promoter” means a nucleic acid sequence sufficient to direct transcription of an operably linked nucleic acid molecule. A promoter can be used together with other transcription control elements (for example, enhancers) that are sufficient to render promoter-dependent gene expression controllable in a cell type-specific, tissue-specific, or temporal-specific manner, or that are inducible by external signals or agents; such elements, may be within the 3′ region of a gene or within an intron. Desirably, a promoter is operably linked to a nucleic acid sequence, for example, a cDNA or a gene sequence, or an effector RNA coding sequence, in such a way as to enable expression of the nucleic acid sequence, or a promoter is provided in an expression cassette into which a selected nucleic acid sequence to be transcribed can be conveniently inserted.

The term “selectable marker” means a polynucleotide segment that allows one to select for or against a molecule or a cell that contains it, often under particular conditions. These markers can encode an activity, such as, but not limited to, production of RNA, peptide, or protein, or can provide a binding site for RNA, peptides, proteins, inorganic and organic compounds or compositions. Examples of selectable markers include but are not limited to: (1) DNA segments that encode products which provide resistance against otherwise toxic compounds (e.g., antibiotics); (2) DNA segments that encode products which are otherwise lacking in the recipient cell (e.g., tRNA genes, auxotrophic markers); (3) DNA segments that encode products which suppress the activity of a gene product; (4) DNA segments that encode products which can be readily identified (e.g., phenotypic markers such as beta-galactosidase, green fluorescent protein (GFP), and cell surface proteins); (5) DNA segments that bind products which are otherwise detrimental to cell survival and/or function; (6) DNA segments that otherwise inhibit the activity of any of the DNA segments described in Nos. 1-5 above (e.g., antisense oligonucleotides); (7) DNA segments that bind products that modify a substrate (e.g. restriction endonucleases); (8) DNA segments that can be used to isolate a desired molecule (e.g. specific protein binding sites); (9) DNA segments that encode a specific nucleotide sequence which can be otherwise non-functional (e.g., for PCR amplification of subpopulations of molecules); and/or (10) DNA segments, which when absent, directly or indirectly confer sensitivity to particular compounds.

Sequence identity can be determined by aligning sequences using algorithms, such as BESTFIT, FASTA, and TFASTA in the Wisconsin Genetics Software Package Release 7.0, Genetics Computer Group, 575 Science Dr., Madison, Wis.), using default gap parameters, or by inspection, and the best alignment (i.e., resulting in the highest percentage of sequence similarity over a comparison window). Percentage of sequence identity is calculated by comparing two optimally aligned sequences over a window of comparison, determining the number of positions at which the identical residues occurs in both sequences to yield the number of matched positions, dividing the number of matched positions by the total number of matched and mismatched positions not counting gaps in the window of comparison (i.e., the window size), and multiplying the result by 100 to yield the percentage of sequence identity. Unless otherwise indicated the window of comparison between two sequences is defined by the entire length of the shorter of the two sequences.

A Sleeping Beauty transposase is a Mariner type transposase with a sequence at least 90, 95, 99 or 100% identical to SEQ ID NO: 28 that is capable of transposing a transposon with left end sequence SEQ ID NO: 24 and right end sequence SEQ ID NO: 25 into the genome of a host cell. A Sleeping Beauty transposon is comprises a left ITR that is at least 90, 95, 99 or 100% identical to SEQ ID NO: 26 and a right ITR that is 90% identical to SEQ ID NO: 27. A Sleeping Beauty transposon may comprise a transposon end (including the ITR) that is at least 90, 95, 99 or 100% identical to SEQ ID NO: 24 and a right transposon end (including the ITR) that is at least 90, 95, 99 or 100% identical to SEQ ID NO: 25, and that can be transposed into the genome of a host cell by the Sleeping Beauty transposase with SEQ ID NO: 28.

A “target nucleic acid” is a nucleic acid into which a transposon is to be inserted. Such a target can be part of a chromosome, episome or vector.

An “integration target sequence” or “target sequence” or “target site” for a transposase is a site or sequence in a target DNA molecule into which a transposon can be inserted by a transposase. The piggyBac transposase from Trichoplusia ni inserts its transposon predominantly into the target sequence 5′-TTAA-3′. PiggyBac-like transposases transpose their transposons using a cut-and-paste mechanism, which results in duplication of their 4 base pair target sequence on insertion into a DNA molecule. The target sequence is thus found on each side of an integrated piggyBac-like transposon.

The term “translation” refers to the process by which a polypeptide is synthesized by a ribosome ‘reading’ the sequence of a polynucleotide.

A ‘transposase’ is a polypeptide that catalyzes the excision of a corresponding transposon from a donor polynucleotide, for example a vector, and (providing the transposase is not integration-deficient) the subsequent integration of the transposon into a target nucleic acid. A transposase may be a piggyBac-like transposase or a mariner transposase such as Sleeping Beauty.

The term “transposition” is used herein to mean the action of a transposase in excising a transposon from one polynucleotide and then integrating it, either into a different site in the same polynucleotide, or into a second polynucleotide.

The term “transposon” means a polynucleotide that can be excised from a first polynucleotide, for instance, a vector, and be integrated into a second position in the same polynucleotide, or into a second polynucleotide, for instance, the genomic or extrachromosomal DNA of a cell, by the action of a corresponding trans-acting transposase. A transposon comprises a first transposon end and a second transposon end, which are polynucleotide sequences recognized by and transposed by a transposase. A transposon usually further comprises a first polynucleotide sequence between the two transposon ends, such that the first polynucleotide sequence is transposed along with the two transposon ends by the action of the transposase. Natural transposons frequently comprise DNA encoding a transposase that acts on the transposon. Transposons of the present invention are “synthetic transposons” comprising a heterologous polynucleotide sequence which is transposable by virtue of its juxtaposition between two transposon ends. A transposon may be a piggyBac-like transposon or a mariner transposon such as Sleeping Beauty.

The term “transposon end” means the cis-acting nucleotide sequences that are sufficient for recognition by and transposition by a corresponding transposase. Transposon ends of piggyBac-like transposons comprise perfect or imperfect repeats such that the respective repeats in the two transposon ends are reverse complements of each other. These are referred to as inverted terminal repeats (ITR) or terminal inverted repeats (TIR). A transposon end may or may not include additional sequence proximal to the ITR that promotes or augments transposition.

The term “vector” or “DNA vector” or “gene transfer vector” refers to a polynucleotide that is used to perform a “carrying” function for another polynucleotide. For example, vectors are often used to allow a polynucleotide to be propagated within a living cell, or to allow a polynucleotide to be packaged for delivery into a cell, or to allow a polynucleotide to be integrated into the genomic DNA of a cell. A vector may further comprise additional functional elements, for example it may comprise a transposon.

An immune cell can refer to any cell of an immune system including cells of adaptive and innate immune systems and including cells of myeloid or lymphoid origin. Examples of immune cells include leucocytes, lymphocytes, macrophages, neutrophils, dendritic cells, lymphoid cells, mast cells eosinophils basophils and natural killer cells. Lymphocytes include B and T lymphocytes. T lymphocytes include killer T cells, helper T cells and gamma delta T cells. Immune cells can be primary cells isolated from a subject or can be the result of further culturing including in the form of a cell line. Immune cells can be the subject of genetic engineering in addition to that described herein, e.g., expression of a CAR-T receptor.

The disclosure refers to several proteins for which it provides an exemplary SEQ ID NO. representing the wildtype human sequence of the protein. Unless otherwise apparent from the context reference to a protein should be understood as including the exemplified SEQ ID NO. as well as allelic, species and induced variants thereof having at least 90, 95, or 99% identity thereto. Examples of allelic and species variants can be found in the SwissProt and other databases. Any such sequences for the protein can be modified to include one or more of the activating mutations described herein to confer enhanced survival of an immune cell expressing the protein as further described herein.

Mutations are sometimes referred to in the form XnY, wherein X is a wildtype amino acid, n is an amino acid position of X in a wildtype sequence, and Y is a replacement amino acid. If the mutation occurs in a sequence having a different number of amino acids than the wildtype sequence, it is present at the position in the sequence aligned with position n in the wildtype sequence when the respective sequences are maximally aligned.

If a nucleic acid is said to encode an activating mutant of a specified protein what is meant is the nucleic acid encodes the protein including the activating mutation.

An apoptosis inhibitor is a substance that interferes with the process of programmed cell death (apoptosis). Apoptosis is a highly regulated process in which cell death is induced by activation of intracellular caspase proteases. Apoptosis inhibitors include proteins whose natural function is to oppose apoptosis, and proteins whose natural function is to participate in apoptosis, but which comprise mutations that interfere with apoptosis.

An apoptosis assay detects and quantifies the cellular events associated with programmed cell death, including caspase activation, cell surface exposure of phosphatidylserine (PS) and DNA fragmentation. The initiator and effector caspases are particularly good targets for detecting apoptosis in cells. Caspase activity assays either use peptide substrates, which are cleaved by caspases, or similar substrates that bind to activated caspases in live cells (McStay et al., 2014 Cold Spring Harbor Protocols, Measuring Apoptosis: Caspase Inhibitors and Activity assays; Niles et al, 2008, Methods Mol Biol., 414:137-50). An exemplary assay to measure apoptosis inhibition is the bioluminescence assay that uses luciferase described herein in paragraph [00174]. A number of caspase assay kits are commercially available that use either fluorescence or luminescence readouts, for example the caspase-Glo® assays from Promega use the luminogenic caspase-8 tetrapeptide substrate (Z-LETD-aminoluciferin), the caspase-9 tetrapeptide substrate (Z-LEHD-aminoluciferin), the caspase-3/7 substrate (Z-DEVD-aminoluciferin), the caspase-6 substrate (Z-VEID-aminoluciferin), or the caspase-2 substrate (Z-VDVAD-aminoluciferin) and a stable luciferase in proprietary buffers. In the absence of active caspase or inhibition of caspase, the caspase substrates do not act as substrates for luciferase and thus produce no light. On cleavage of the substrates by the respective caspase, aminoluciferin is liberated and can contribute to the generation of light in a luminescence reaction. The resulting luminescent signal is directly proportional to the amount of caspase activity present in the sample. An example of a caspase activity assay kit that uses a fluorescence substrate N-AcetylAsp-Glu-Val-Asp-7-amino-4-methylcoumarin or Ac-DEVDAMC for caspase-3 is the Caspase-3 Activity assay kit from Cell Signaling Technology. Activated caspase-3 cleaves this substrate between DEVD and AMC, generating highly fluorescent AMC that can be detected using a fluorescence reader with excitation at 380 nm and emission between 420-460 nm. Cleavage of the substrate only occurs in lysates of apoptotic cells; therefore, the amount of AMC produced is proportional to the number of apoptotic cells in the sample.

### Genetic Elements Useful for Expression in Immune Cells

### Transposon Elements

The consistency of expression of a gene from a heterologous polynucleotide in an immune cell can be improved if the heterologous polynucleotide is integrated into the genome of the host cell. Integration of a polynucleotide into the genome of a host cell also generally makes it stably heritable, by subjecting it to the same mechanisms that ensure the replication and division of genomic DNA. Such stable heritability is desirable for achieving good and consistent expression over long growth periods. For stable modification of immune cells, particularly for therapeutic applications, the stability of the modification and consistency of expression levels are important.

Heterologous polynucleotides may be more efficiently integrated into a target genome if they are part of a transposon, for example so that they may be integrated by a transposase. A particular benefit of a transposon is that the entire polynucleotide between the transposon ITRs is integrated. This is in contrast with random integration, where a polynucleotide introduced into a eukaryotic cell is often fragmented at random in the cell, and only parts of the polynucleotide become incorporated into the target genome, usually at a low frequency. Heterologous polynucleotides incorporated into piggyBac-like transposons may be integrated into immune cells, as well as hepatocytes, neural cells, muscle cells, blood cells, embryonic stem cells, somatic stem cells, hematopoietic cells, embryos, zygotes and sperm cells (some of which are open to be manipulated in an in vitro setting). Preferred cells can also be pluripotent cells (cells whose descendants can differentiate into several restricted cell types, such as hematopoietic stem cells or other stem cells) or totipotent cells (i.e., a cell whose descendants can become any cell type in an organism, e.g., embryonic stem cells).

Preferred gene transfer systems comprise a transposon in combination with a corresponding transposase protein that transposases the transposon, or a nucleic acid that encodes the corresponding transposase protein and is expressible in the target cell. piggyBac-like transposons are advantageous as gene transfer systems for the applications described herein compared with lentiviral vectors for several reasons. Lentiviruses are not packaged efficiently if they exceed a certain size, and a significant amount of their DNA is already occupied with sequences required for viral synthesis, assembly and packaging. Genes integrated through lentiviral vectors can show highly variable expression due to promoter silencing (Antoniou et al., 2013. Hum Gene Ther 24, 363-374. “Optimizing retroviral gene expression for effective therapies”): silencing can be reduced either by increasing copy number or by incorporating insulators into the integrating polynucleotide (Emery, 2011. Hum Gene Ther 22, 761-774. “The use of chromatin insulators to improve the expression and safety of integrating gene transfer vectors”). Including insulators in lentiviral constructs can be challenging because of size limitations and because of effects of including these sequences on viral packaging and titer. In contrast the efficient integration of a piggyBac-like transposon into a target genome by its corresponding transposase is unperturbed by increasing the transposon size. It is therefore possible to include multiple genes for modification of the properties of an immune cell into a single transposon, together with flanking insulators, without compromising the ability of the corresponding transposase to integrate the transposon into the genome of an immune cell. Safety is also of significant concern when modifying the genome of a cell that is to be placed into a human. When making modifications of immune cells such as T-cells to enhance their ability to kill tumor cells and to improve their ability to survive and proliferate, it is therefore useful to be able to also incorporate into the genome of the cell a gene that provides a means of killing the modified immune cell. Examples of such “kill switches” include expression of an antigen that is efficiently recognized by an existing therapeutic agent (for example a surface-expressed antigen such as CD20 that is normally found exclusively on B-cells and is recognized and treated by the drug rituximab or CD19 that is normally found exclusively on B-cells and is recognized and treated by the drug blinotumomab) and an inducible caspase 9 suicide switch (Straathof et. al., 2005. Blood 105, 4247-4254. “An inducible caspase 9 safety switch for T-cell therapy”). For kill switches to be useful, they must be present in the genome of every modified cell. An example of an attempt to do this in a lentiviral vector carrying a gene for a chimeric antigen receptor plus sequences encoding a CD19 selectable marker and an inducible caspase 9 as a kill switch is described by Budde et al (PLoS One 8(12): e82742. doi: 10.1371/journal.pone.0082742. eCollection 2013. “Combining a CD20 chimeric antigen receptor and an inducible caspase 9 suicide switch to improve the efficacy and safety of T cell adoptive immunotherapy for lymphoma.”). In order to combine kill switches with the chimeric antigen receptor gene, the authors had to use viral CHYSL/2A sequences to separate the polypeptide comprising the chimeric antigen receptor from the inducible caspase 9, and they had to truncate the CD19 gene. This cargo plus the regulatory elements for expression occupied essentially the entire capacity of the lentiviral vector, leaving no additional space for the addition of insulators or for other genes such as those for enhancing the survival or proliferation or function of the T-cell. Gene transfer systems comprising a piggyBac-like transposon and its corresponding transposase are thus advantageous for integrating genes including genes encoding chimeric antigen receptors into the genomes of immune cells including T-cells.

A Xenopus transposon is an advantageous piggyBac-like transposon for modifying the genome of an immune cell and comprises an ITR with the with sequence given by SEQ ID NO: 6, a heterologous polynucleotide to be transposed and a second ITR with sequence given by SEQ ID NO: 7. The transposon may further be flanked by a copy of the tetranucleotide 5′-TTAA-3′ on each side, immediately adjacent to the ITRs and distal to the heterologous polynucleotide. The transposon may further comprise a sequence immediately adjacent to the ITR and proximal to the heterologous polynucleotide that is at least 95% identical to SEQ ID NO: 1 or 2 on one side of the heterologous polynucleotide, preferably the left side, and a sequence immediately adjacent to the ITR and proximal to the heterologous polynucleotide that is at least 95% identical to SEQ ID NO: 4 or 5 on the other side of the heterologous polynucleotide, preferably the right side. This transposon may be transposed by a transposase comprising a sequence at least 90% identical to the sequence given by SEQ ID 31 or 32, for example any of SEQ ID NOs: 33-63. Preferably the transposase is a hyperactive variant of a naturally occurring transposase. Preferably the hyperactive variant transposase comprises one of the following amino acid changes, relative to the sequence of SEQ ID NO: 31: Y6L, Y6H, Y6V, Y61, Y6C, Y6G, Y6A, Y6S, Y6F, Y6R, Y6P, Y6D, Y6N, S7G, S7V, S7D, E9W, E9D, E9E, M16E, M16N, M16D, M16S, M16Q, M16T, M16A, M16L, M16H, M16F, M161, S18C, 518Y, S18M, S18L, S18Q, S18G, S18P, S18A, S18W, S18H, S18K, S18I, S18V, S19C, S19V, S19L, S19F, S19K, S19E, S19D, S19G, S19N, S19A, S19M, S19P, 519Y, S19R, S19T, S19Q, 520G, 520M, S20L, 520V, S20H, S20W, 520A, 520C, 520Q, 520D, 520F, S20N, S20R, E21N, E21W, E21G, E21Q, E21L, E21D, E21A, E21P, E21T, E21S, E21Y, E21V, E21F, E21M, E22C, E22H, E22R, E22L, E22K, E22S, E22G, E22M, E22V, E22Q, E22A, E22Y, E22W, E22D, E22T, F23Q, F23A, F23D, F23W, F23K, F23T, F23V, F23M, F23N, F23P, F23H, F23E, F23C, F23R, F23Y, S24L, S24W, S24H, S24V, S24P, S241, S24F, S24K, S24Y, S24D, S24C, S24N, S24G, S24A, S26F, S26H, S26V, S26Q, S26Y, S26W, S28K, S28Y, S28C, S28M, S28L, S28H, S28T, S28Q, V31L, V31T, V311, V31Q, V31K, A34L, A34E, L67A, L67T, L67M, L67V, L67C, L67H, L67E, L67Y, G73H, G73N, G73K, G73F, G73V, G73D, G73S, G73W, G73L, A76L, A76R, A76E, A761, A76V, D77N, D77Q, D77Y, D77L, D77T, P88A, P88E, P88N, P88H, P88D, P88L, N91D, N91R, N91A, N91L, N91H, N91V, Y1411, Y141M, Y141Q, Y141S, Y141E, Y141W, Y141V, Y141F, Y141A, Y141C, Y141K, Y141L, Y141H, Y141R, N145C, N145M, N145A, N145Q, N145I, N145F, N145G, N145D, N145E, N145V, N145H, N145W, N145Y, N145L, N145R, N145S, P146V, P146T, P146W, P146C, P146Q, P146L, P146Y, P146K, P146N, P146F, P146E, P148M, P148R, P148V, P148F, P148T, P148C, P148Q, P148H, Y150W, Y150A, Y150F, Y150H, Y150S, Y150V, Y150C, Y150M, Y150N, Y150D, Y150E, Y150Q, Y150K, H157Y, H157F, H157T, H157S, H157W, A162L, A162V, A162C, A162K, A162T, A162G, A162M, A162S, A1621, A162Y, A162Q, A179T, A179K, A179S, A179V, A179R, L182V, L1821, L182Q, L182T, L182W, L182R, L182S, T189C, T189N, T189L, T189K, T189Q, T189V, T189A, T189W, T189Y, T189G, T189F, T189S, T189H, L192V, L192C, L192H, L192M, L1921, S193P, S193T, S193R, S193K, S193G, S193D, S193N, S193F, S193H, S193Q, S193Y, V196L, V196S, V196W, V196A, V196F, V196M, V1961, S198G, S198R, S198A, S198K, T200C, T200I, T200M, T200L, T200N, T200W, T200V, T200Q, T200Y, T200H, T200R, S202A, S202P, L210H, L210A, F212Y, F212N, F212M, F212C, F212A, N218V, N218R, N218T, N218C, N218G, N218I, N218P, N218D, N218E, A248S, A248L, A248H, A248C, A248N, A2481, A248Q, A248Y, A248M, A248D, L263V, L263A, L263M, L263R, L263D, Q270V, Q270K, Q270A, Q270C, Q270P, Q270L, Q2701, Q270E, Q270G, Q270Y, Q270N, Q270T, Q270W, Q270H, S294R, S294N, S294G, S294T, S294C, T297C, T297P, T297V, T297M, T297L, T297D, E304D, E304H, E304S, E304Q, E304C, S308R, S308G, L310R, L3101, L310V, L333M, L333W, L333F, Q336Y, Q336N, Q336M, Q336A, Q336T, Q336L, Q3361, Q336G, Q336F, Q336E, Q336V, Q336C, Q336H, A354V, A354W, A354D, A354C, A354R, A354E, A354K, A354H, A354G, C357Q, C357H, C357W, C357N, C3571, C357V, C357M, C357R, C357F, C357D, L358A, L358F, L358E, L358R, L358Q, L358V, L358H, L358C, L358M, L358Y, L358K, L358N, L3581, D359N, D359A, D359L, D359H, D359R, D359S, D359Q, D359E, D359M, L377V, L3771, V423N, V423P, V423T, V423F, V423H, V423C, V423S, V423G, V423A, V423R, V423L, P426L, P426K, P426Y, P426F, P426T, P426W, P426V, P426C, P426S, P426Q, P426H, P426N, K428R, K428Q, K428N, K428T, K428F, S434A, S434T, S438Q, S438A, S438M, T447S, T447A, T447C, T447Q, T447N, T447G, L450M, L450V, L450A, L450I, L450E, A462M, A462T, A462Y, A462F, A462K, A462R, A462Q, A462H, A462E, A462N, A462C, V467T, V467C, V467A, V467K, I469V, I469N, I472V, I472L, I472W, I472M, I472F, L476I, L476V, L476N, L476F, L476M, L476C, L476Q, P488E, P488H, P488K, P488Q, P488F, P488M, P488L, P488N, P488D, Q498V, Q498L, Q498G, Q498H, Q498T, Q498C, Q498E, Q498M, L5021, L502M, L502V, L502G, L502F, E517M, E517V, E517A, E517K, E517L, E517G, E517S, E517I, P520W, P520R, P520M, P520F, P520Q, P520V, P520G, P520D, P520K, P520Y, P520E, P520L, P520T, S521A, S521H, S521C, S521V, S521W, S521T, S521K, S521F, S521G, N523W, N523A, N523G, N523S, N523P, N523M, N523Q, N523L, N523K, N523D, N523H, N523F, N523C, I533M, I533V, I533T, I533S, I533F, I533G, 1533E, D534E, D534Q, D534L, D534R, D534V, D534C, D534M, D534N, D534A, D534G, D534F, D534T, D534H, D534K, D534S, F576L, F576K, F576V, F576D, F576W, F576M, F576C, F576R, F576Q, F576A, F576Y, F576N, F576G, F576I, F576E, K577L, K577G, K577D, K577R, K577H, K577Y, K577I, K577E, K577V, K577N, I582V, I582K, I582R, I582M, I582G, I582N, 1582E, I582A, I582Q, Y583L, Y583C, Y583F, Y583D, Y583Q, L587F, L587D, L587R, L587I, L587P, L587N, L587E, L587S, L587Y, L587M, L587Q, L587G, L587W, L587K or L587T.

A Bombyx transposon is an advantageous piggyBac-like transposon for modifying the genome of an immune cell and comprises an ITR with the sequence of SEQ ID NO: 14, a heterologous polynucleotide to be transposed and a second ITR with the sequence of SEQ ID NO: 15. The transposon may further be flanked by a copy of the tetranucleotide 5′-TTAA-3′ on each side, immediately adjacent to the ITRs and distal to the heterologous polynucleotide. The transposon may further comprise a sequence immediately adjacent to the ITR and proximal to the heterologous polynucleotide that is at least 95% identical to SEQ ID NO: 12 on one side of the heterologous polynucleotide, preferably the left side, and a sequence immediately adjacent to the ITR and proximal to the heterologous polynucleotide that is at least 95% identical to SEQ ID NO: 13 on the other side of the heterologous polynucleotide, preferably the right side. This transposon may be transposed by a transposase comprising a sequence at least 90% identical to SEQ ID NO: 64, for example any of SEQ ID NOs: 65-86. Preferably the transposase is a hyperactive variant of a naturally occurring transposase. Preferably the hyperactive variant transposase comprises one of the following amino acid changes, relative to the sequence of SEQ ID NO: BM-Tpase1: Q85E, Q85M, Q85K, Q85H, Q85N, Q85T, Q85F, Q85L, Q92E, Q92A, Q92P, Q92N, Q92I, Q92Y, Q92H, Q92F, Q92R, Q92D, Q92M, Q92W, Q92C, Q92G, Q92L, Q92V, Q92T, V93P, V93K, V93M, V93F, V93W, V93L, V93A, V93I, V93Q, P96A, P96T, P96M, P96R, P96G, P96V, P96E, P96Q, P96C, F97Q, F97K, F97H, F97T, F97C, F97W, F97V, F97E, F97P, F97D, F97A, F97R, F97G, F97N, F97Y, H165E, H165G, H165Q, H165T, H165M, H165V, H165L, H165C, H165N, H165D, H165K, H165W, H165A, E178S, E178H, E178Y, E178F, E178C, E178A, E178Q, E178G, E178V, E178D, E178L, E178P, E178W, C189D, C189Y, C1891, C189W, C189T, C189K, C189M, C189F, C189P, C189Q, C189V, A196G, L200I , L200F, L200C, L200M, L200Y, A201Q, A201L, A201M, L203V, L203D, L203G, L203E, L203C, L203T, L203M, L203A, L203Y, N207G, N207A, L211G, L211M, L211C, L211T, L211V, L211A, W215Y, T217V, T217A, T217I, T217P, T217C, T217Q, T217M, T217F, T217D, T217K, G219S, G219A, G219C, G219H, G219Q, Q235C, Q235N, Q235H, Q235G, Q235W, Q235Y, Q235A, Q235T, Q235E, Q235M, Q235F, Q238C, Q238M, Q238H, Q238V, Q238L, Q238T, Q2381, R242Q, K2461, K253V, M258V, F261L, S263K, C271S, N303C, N303R, N303G, N303A, N303D, N303S, N303H, N303E, N303R, N303K, N303L, N303Q, 1312F, 1312C, 1312A, 1312L, 1312T, 1312V, 1312G, 1312M, F321H, F321R, F321N, F321Y, F321W, F321D, F321G, F321E, F321M, F321K, F321A, F321Q, V3231, V323L, V323T, V323M, V323A, V324N, V324A, V324C, V3241, V324L, V324T, V324K, V324Y, V324H, V324F, V324S, V324Q, V324M, V324G, A330K, A330V, A330P, A330S, A330C, A330T, A330L, Q333P, Q333T, Q333M, Q333H, Q333S, P337W, P337E, P337H, P3371, P337A, P337M, P337N, P337D, P337K, P337Q, P337G, P337S, P337C, P337L, P337V, F368Y, L373C, L373V, L3731, L373S, L373T, V3891, V389M, V389T, V389L, V389A, R394H, R394K, R394T, R394P, R394M, R394A, Q395P, Q395F, Q395E, Q395C, Q395V, Q395A, Q395H, Q395S, Q395Y, S399N, S399E, S399K, S399H, S399D, S399Y, S399G, S399Q, S399R, S399T, S399A, S399V, S399M, R402Y, R402K, R402D, R402F, R402G, R402N, R402E, R402M, R402S, R402Q, R402T, R402C, R402L, R402V, T403W, T403A, T403V, T403F, T403L, T403Y, T403N, T403G, T403C, T4031, T403S, T403M, T403Q, T403K, T403E, D404I, D404S, D404E, D404N, D404H, D404C, D404M, D404G, D404A, D404Q, D404L, D404P, D404V, D404W, D404F, N408F, N408I, N408A, N408E, N408M, N408S, N408D, N408Y, N408H, N408C, N408Q, N408V, N408W, N408L, N408P, N408K, S409H, S409Y, S409N, S409I, S409D, S409F, S409T, S409C, S409Q, N441F, N441R, N441M, N441G, N441C, N441D, N441L, N441A, N441V, N441W, G448W, G448Y, G448H, G448C, G448T, G448V, G448N, G448Q, E449A, E449P, E449T, E449L, E449H, E449G, E449C, E449I, V469T, V469A, V469H, V469C, V469L, L472K, L472Q, L472M, C473G, C473Q, C473T, C473I, C473M, R484H, R484K, T507R, T507D, T507S, T507G, T507K, T507I, T507M, T507E, T507C, T507L, T507V, G523Q, G523T, G523A, G523M, G523S, G523C, G523I, G523L, I527M, I527V, Y528N, Y528W, Y528M, Y528Q, Y528K, Y528V, Y528I, Y528G, Y528D, Y528A, Y528E, Y528R, Y543C, Y543W, Y543I, Y543M, Y543Q, Y543A, Y543R, Y543H, E549K, E549C, E549I, E549Q, E549A, E549H, E549C, E549M, E549S, E549F, E549L, K550R, K550M, K550Q, S556G, S556V, S556I, P557W, P557T, P557S, P557A, P557Q, P557K, P557D, P557G, P557N, P557L, P557V, H559K, H559S, H559C, H559I, H559W, V560F, V560P, V560I, V560H, V560Y, V560K, N561P, N561Q, N561G, N561A, V562Y, V562I, V562S, V562M, V567I, V567H, V567N, S583M, E601V, E601F, E601Q, E601W, E605R, E605W, E605K, E605M, E605P, E605Y, E605C, E605H, E605A, E605Q, E605S, E605V, E6051, E605G, D607V, D607Y, D607C, D607N, D607W, D607T, D607A, D607H, D607Q, D607E, D607L, D607K, D607G, S609R, S609W, S609H, S609V, S609Q, S609G, S609T, S609K, S609N, S609Y, L610T, L610I, L610K, L610G, L610A, L610W, L610D, L610Q, L610S, L610F or L610N.

A piggyBat transposon is an advantageous piggyBac-like transposon for modifying the genome of an immune cell and comprises an ITR with the sequence of SEQ ID NO: 20, a heterologous polynucleotide to be transposed and a second ITR with the sequence of SEQ ID NO: 21. The transposon may further be flanked by a copy of the tetranucleotide 5′-TTAA-3′ on each side, immediately adjacent to the ITRs and distal to the heterologous polynucleotide. The transposon may further comprise a sequence immediately adjacent to the ITR and proximal to the heterologous polynucleotide that is at least 95% identical to SEQ ID NO: 22 on one side of the heterologous polynucleotide, preferably the left side, and a sequence immediately adjacent to the ITR and proximal to the heterologous polynucleotide that is at least 95% identical to SEQ ID NO: 23 on the other side of the heterologous polynucleotide, preferably the right side. This transposon may be transposed by a transposase comprising a sequence at least 90% identical to SEQ ID NO: 29. Preferably the transposase is a hyperactive variant of a naturally occurring transposase. Preferably the hyperactive variant transposase comprises one of the following amino acid changes, relative to the sequence of SEQ ID NO: 29: A14V, D475G, P491Q, A561T, T546T, T300A, T294A, A520T, C239S, S5P, S8, S54N, D9N, D9G. 1345 V, M481V, E11G, K130T G9G, R427H, S8P, S36G D1OG, S36G.

An advantageous piggyBac-like transposon for modifying the genome of an immune cell comprises an ITR with the sequence of SEQ ID NO: 16, a heterologous polynucleotide to be transposed and a second ITR with the sequence of SEQ ID NO: 17. The transposon may further be flanked by a copy of the tetranucleotide 5′-TTAA-3′ on each side, immediately adjacent to the ITRs and distal to the heterologous polynucleotide. The transposon may further comprise a sequence immediately adjacent to the ITR and proximal to the heterologous polynucleotide that is at least 95% identical to SEQ ID NO: 18 on one side of the heterologous polynucleotide, preferably the left side, and a sequence immediately adjacent to the ITR and proximal to the heterologous polynucleotide that is at least 95% identical to SEQ ID NO: 19 on the other side of the heterologous polynucleotide preferably the right side. This transposon may be transposed by a transposase comprising a sequence at least 90% identical to SEQ ID NO: 30. Preferably the transposase is a hyperactive variant of a naturally occurring transposase. Preferably the hyperactive variant transposase comprises one of the following amino acid changes, relative to the sequence of SEQ ID NO: 30: G2C, Q40R, I30V, G1655, T43A, S61R, S103P, S103T, M194V, R281G, M282V, G316E, I426V, Q497L, N505D, Q573L, 5509G, N570S, N538K, Q591P, Q591R, F594L, M194V, 130V, S103P, G1655, M282V, 5509G, N538K, N571S, C41T, A1424G, C1472A, G1681A, T150C, A351G, A279G, T1638C, A898G, A880G, G1558A, A687G, G715A, T13C, C23T, G161A, G25A, T1050C, A1356G, A26G, A1033G, A1441G, A32G, A389C, A32G, A389C, A32G, T1572A, G456A, T1641C, Tl 155C, G1280A, T22C, A106G, A29G, C137T, A14V, D475G, P491Q, A561T, T546T, T300A, T294A, A520T, G239S, S5P, S8F, S54N, D9N, D9G, 1345 V, M481V, E11G, K130T, G9G, R427H, S8P, S36G, D10G, S36G, A51T, C153A, C277T, G201A, G202A, T236A, A103T, A104C, T140C, G138T, T118A, C74T, A179C, S3N, I30V, A465, A46T, I82W, S103P, R119P, C125A, C125L, G1655, Y177K, Y177H, F180L, F1801, F180V, M185L, A187G, F200W, V207P, V209F, M226F, L235R, V240K, F241L, P243K, N258S, M282Q, L296W, L296Y, L296F, M298V, M298A, M298L, P311V, P311I, R315K, T319G, Y327R, Y328V, C340G, C340L, D421H, V436I, M456Y, L470F, S486K, M5031, M503L, V552K, A570T, Q591P, Q591R, R65A, R65E, R95A, R95E, R97A, R97E, R135A, R135E, R161A, R161E, R192A, R192E, R208A, R208E, K176A, K176E, K195A, K195E, S171E, M14V, D270N, 130V, G1655, M282L, M2821, M282V or M282A.

An advantageous Mariner transposon for modifying the genome of an immune cell is a Sleeping Beauty transposon which comprises an ITR with the sequence of SEQ ID NO: 26, a heterologous polynucleotide and a second ITR with the sequence of SEQ ID NO: 27. The ITR may be part of a longer transposon end sequence, for example the transposon may comprise a left end with a sequence at least 95% identical to SEQ ID NO: 24 and a right end with sequence at least 95% identical to SEQ ID NO: 25. This transposon may be transposed by a transposase comprising a sequence at least 90% identical to SEQ ID NO: 28, including hyperactive variants thereof.

An advantageous hAT transposon for modifying the genome of an immune cell is a TcBuster transposon which comprises an ITR with the sequence of SEQ ID NO: 399, a heterologous polynucleotide and a second ITR with the sequence of SEQ ID NO: 400. The ITR may be part of a longer transposon end sequence, for example the transposon may comprise a left end with a sequence at least 95% identical to SEQ ID NO: 397 and a right end with sequence at least 95% identical to SEQ ID NO: 398. This transposon may be transposed by a transposase comprising a sequence at least 90% identical to SEQ ID NO: 401, including hyperactive variants thereof.

A transposase protein can be introduced into a cell as a protein or as a nucleic acid encoding the transposase, for example as a ribonucleic acid, including mRNA or any polynucleotide recognized by the translational machinery of a cell; as DNA, e.g. as extrachromosomal DNA including episomal DNA; as plasmid DNA, or as viral nucleic acid. Furthermore, the nucleic acid encoding the transposase protein can be transfected into a cell as a nucleic acid vector such as a plasmid, or as a gene expression vector, including a viral vector. The nucleic acid can be circular or linear. DNA encoding the transposase protein can be stably inserted into the genome of the cell or into a vector for constitutive or inducible expression. Where the transposase protein is transfected into the cell or inserted into the vector as DNA, the transposase encoding sequence is preferably operably linked to a heterologous promoter. There are a variety of promoters that could be used including constitutive promoters, tissue-specific promoters, inducible promoters, and the like. All DNA or RNA sequences encoding piggyBac-like transposase proteins are expressly contemplated. Alternatively, the transposase may be introduced into the cell directly as protein, for example using cell-penetrating peptides (e.g. as described in Ramsey and Flynn (2015) Pharmacol. Ther. 154: 78-86 “Cell-penetrating peptides transport therapeutics into cells”); using small molecules including salt plus propanebetaine (e.g. as described in Astolfo et al (2015) Cell 161: 674-690); or electroporation (e.g. as described in Morgan and Day (1995) Methods in Molecular Biology 48: 63-71 “The introduction of proteins into mammalian cells by electroporation”).

### Gene Transfer Systems

Gene transfer systems comprise a polynucleotide to be transferred to a host cell. The gene transfer system may comprise any of the transposons or transposases described herein, or it may comprise one or more polynucleotides that have other features that facilitate efficient gene transfer without the need for a transposase or transposon.

When there are multiple components of a gene transfer system, for example the one or more polynucleotides comprising genes for expression in the target cell and optionally comprising transposon ends, and a transposase (which may be provided either as a protein or encoded by a nucleic acid), these components can be transfected into a cell at the same time, or at different times. For example, a transposase protein or its encoding nucleic acid may be transfected into a cell prior to, simultaneously with or subsequently to transfection of a corresponding transposon. Additionally, administration of either component of the gene transfer system may occur repeatedly, for example, by administering at least two doses of this component.

Transposase proteins may be encoded by polynucleotides including RNA or DNA. If the transposase is provided as a gene encoded in DNA, it should preferably be operably linked to a promoter that is active in the target cell. Preferable RNA molecules include those with appropriate substitutions to reduce toxicity effects on the cell, for example substitution of uridine with pseudouridine, and substitution of cytosine with 5-methyl cytosine. Similarly, the transposon or the nucleic acid encoding the transposase of this invention can be transfected into the cell as a linear fragment or as a circularized fragment, either as a plasmid or as recombinant viral DNA.

The components of the gene transfer system may be transfected into one or more cells by techniques such as particle bombardment, electroporation, microinjection, combining the components with lipid nanoparticles or lipid-containing vesicles, such as cationic lipid vesicles, DNA condensing reagents (example, calcium phosphate, polylysine or polyethyleneimine), or inserting the components (that is the nucleic acids) thereof into a viral vector and contacting the viral vector with the cell. Where a viral vector is used, the viral vector can include any of a variety of viral vectors known in the art including viral vectors selected from the group consisting of a retroviral vector, an adenovirus vector or an adeno-associated viral vector. The gene transfer system may be formulated in a suitable manner as known in the art, or as a pharmaceutical composition or kit.

### Promoter Elements

Gene transfer systems for expression of polypeptides in immune cells comprise a polynucleotide to be transferred to a host cell. The polynucleotide comprises a promoter that is active in the immune cell. Examples include promoters from constitutively expressed genes including mammalian glyceraldehyde 3-phosphate dehydrogenase (GAPDH) genes (for example sequences given by SEQ ID NOs: 97-107), mammalian phosphoglycerate kinase (PGK) genes (for example sequences given by SEQ ID NOs: 115-118), mammalian elongation factor 1a (EF1a) genes (for example sequences given by SEQ ID NOs: 94, 96 and 128-146), mammalian elongation factor 2 (EEF2) genes (for example sequences given by SEQ ID NOs: 1108, 109, 114 and 147-154) and ubiquitin genes (for example sequences given by SEQ ID NO: 95 or 125-127). These genes may be used with or without intron sequences including their natural intron sequences. Exemplary intron sequences are given as SEQ ID NOs: 155-159.

### Polyadenylation Elements

Gene transfer systems are useful for introducing genes for expression into eukaryotic cells. Many eukaryotic cells, including animal cells and higher plant cells, process the mRNA transcribed during gene expression. Protein-encoding genes are often polyadenylated, which stabilizes the mRNA within the cell. Polyadenylation signals may also help to terminate transcription. This can be particularly useful when more than one open reading frame is to be expressed from a polynucleotide, as it helps to reduce interference between two promoters. Polyadenylation sequences that are effective at terminating transcription from one promoter, thereby reducing interference with a second promoter located to the 3′ of the first promoter may be designed synthetically. Sequences SEQ ID NOs: 160-217 are all useful for initiating polyadenylation of a transcribed sequence, and in terminating transcription. Polyadenylation sequences SEQ ID NOs: 160-217 may be included in the polynucleotide of a gene transfer system for expression of genes in animal cells including vertebrate or invertebrate cells. Polyadenylation sequences SEQ ID NOs: 160-217 are useful for expressing genes in vertebrate cells including cells from mammals including rodents such as rats, mice, and hamsters; ungulates, such as cows, goats or sheep; swine; cells from human tissues and human stem cells. Polyadenylation sequences SEQ ID NOs: 160-217 are useful in different cell types including immune cells, lymphocytes, hepatocytes, neural cells, muscle cells, blood cells, embryonic stem cells, somatic stem cells, hematopoietic cells, embryos, zygotes and sperm cells (some of which are open to be manipulated in an in vitro setting). Polyadenylation sequences SEQ ID NOs: 160-217 are useful for expressing genes in pluripotent cells (cells whose descendants can differentiate into several restricted cell types, such as hematopoietic stem cells or other stem cells) or totipotent cells (i.e., a cell whose descendants can become any cell type in an organism, e.g., embryonic stem cells). Polyadenylation sequences SEQ ID NOs: 160-217 are useful for expressing genes in culture cells such as Chinese hamster ovary (CHO) cells or Human embryonic kidney (HEK293) cells.

Polyadenylation sequences SEQ ID NOs: 160-217 may be incorporated into a piggyBac-like transposon, or a Mariner transposon such as a Sleeping Beauty transposon, or an hAT transposon such as TcBuster, or in a non-transposon-based gene delivery polynucleotide. Polyadenylation sequences SEQ ID NOs: 160-217 are preferably incorporated into a polynucleotide to the 3′ end of an open reading frame to be expressed. Polyadenylation sequences SEQ ID NOs: 160-217 are useful when placed between two genes to be expressed, to terminate transcription from a first promoter and reduce promoter interference. An advantageous gene transfer system comprises a sequence at least 80% or 90% or 95% or 96% or 97% or 98% or 99% or 100% identical to any of SEQ ID NOs: 160-217.

### Insulator Elements

When a heterologous polynucleotide is integrated into the genome of an immune cell, it is often desirable to prevent genetic elements within the heterologous polynucleotide from influencing expression of endogenous immune cell genes. Similarly, it is often desirable to prevent genes within the heterologous polynucleotide from being influenced by elements in the immune cell genome, for example from being silenced by incorporation into heterochromatin. Insulator elements are known to have enhancer-blocking activity (helping to prevent the genes in the heterologous polynucleotide from influencing the expression of endogenous immune cell genes) and barrier activity (helping to prevent genes within the heterologous polynucleotide from being silenced by incorporation into heterochromatin). Enhancer-blocking activity can result from binding of transcriptional repressor CTCF protein. Barrier activity can result from binding of vertebrate barrier proteins such as USF1 and VEZF1. Useful insulator sequences comprise binding sites for CTCF, USF1 or VEZF1. An advantageous gene transfer system comprises a polynucleotide comprising an insulator sequence comprising a binding site for CTCF, USF1 or VEZF1. More preferably a gene transfer system comprises a polynucleotide comprising two insulator sequences, each comprising a binding site for CTCF, USF1 or VEZF1, wherein the two insulator sequences flank any promoters or enhancers within the heterologous polynucleotide. Advantageous examples of insulator sequences are given as SEQ ID NOs: 87-93.

If a heterologous polynucleotide comprising a promoter or enhancer is integrated into the genome of an immune cell without insulator sequences, there is a risk that either the promoter or enhancer elements within the heterologous polynucleotide will influence expression of endogenous immune cell genes (for example oncogenes), or that promoter or enhancer elements within the heterologous polynucleotide will be silenced by incorporation into heterochromatin. When a heterologous polynucleotide is integrated into a target genome following random fragmentation, some genetic elements are often lost, and others may be rearranged. There is thus a significant risk that, if the heterologous polynucleotide comprises insulator elements flanking enhancer and promoter elements, the insulator elements may be rearranged or lost, and the enhancer and promoter elements may be able to influence and be influenced by the genomic environment into which they integrate. It is therefore advantageous to use a transposon gene transfer system, wherein the entire sequence between the two transposon ITRs is integrated, without rearrangement, into the immune cell genome. Advantageous gene transfer systems for integration into immune cell genomes thus comprise a transposon in which elements are arranged in the following order: left transposon end; a first insulator sequence; sequences for expression within the immune cell; a second insulator sequence; right transposon end. The sequences for expression within the immune cell may include any number of regulatory sequences operably linked to any number of open reading frames. The transposon ends are preferably those of a piggyBac-like transposon or a Mariner transposon such as a Sleeping Beauty transposon, or a hAT transposon such as TcBuster transposon.

### Genetic Elements Useful for Enhancing Immune Cell Survival

For immune cells to respond adequately to threats to the body, they must be able to survive for long enough to attack their targets. For therapies and research that require the ex vivo manipulation of immune cells, it is advantageous for the immune cells to proliferate. However, neither ex vivo culture conditions nor certain in vivo environments (for example the environment within a solid tumor) are optimal for growth of immune cells. For example, T-cells from heavily pre-treated lymphoma patients show lower rates of ex vivo expansion and clinical response when engineered with anti-CD19 chimeric antigen receptor than T-cells from untreated patients. There is therefore a need for methods that enhance the function, persistence and proliferation of human immune cells, particularly under conditions that are naturally hostile to the immune cells.

### T-Cell Transformation Elements

One approach to enhance the persistence and proliferation of human immune cells is to integrate genetic elements to increase growth and/or survival into the genome of the immune cell. Candidate genetic elements for enhancing immune cell survival include genes found to be mutated in immune cell cancers. However, transformation of a cell into a cancer cell is typically thought to require a series of mutations, and the role of each mutation may not be directly related to cell survival or growth. For example, many mutations are known to simply increase the chance that additional mutations will occur. Thus, even though there may be correlations whereby mutations in certain genes often occur in immune cell cancers, it is generally not the case that introducing that same mutated gene into an immune cell will enhance the growth or survival of that cell. Testing can therefore be performed to determine whether integration into the genome of an immune cell, of a heterologous polynucleotide comprising a gene comprising naturally occurring mutations will increase the survival and proliferation of that cell.

We sought to identify genes that can be provided on a heterologous polynucleotide and integrated into the genome of an immune cell, to confer upon that immune cell a growth or survival benefit. To do this we synthesized polynucleotides comprising genes having a sequence encoding a naturally occurring mutant human protein including an activating mutation operably linked to a heterologous promoter effective for expression of the protein in an immune cell, and integrated these heterologous polynucleotides into the genomes of T-cells. We then measured the growth and survival of these T-cells in ex vivo culture, as described in Section 6.2.

### STAT3

The gene encoding STAT3 (signal transducer and activator of transcription 3) is often found to be mutated in large granular lymphocytic leukemia. These activating mutations are frequently in the SH2 domain of STAT3, and include S614R, E616K, G618R, Y640F, N6471, E652K, K658Y, K658R, K658N, K658M, K658R, K658H, K658N, D661Y and D661V. Activating mutations in STAT3 have also been found outside the SH2 domain, for example F174S and H410R. As described in Sections 6.2.1.1 and 6.2.1.5, we have demonstrated that a heterologous polynucleotide encoding an activating mutant of a STAT3 protein may be introduced into an immune cell to enhance its survival or its proliferation; a gene encoding an activating mutant of STAT3 is an immune cell survival-enhancing gene and an immune cell proliferation-enhancing gene as described in Section 6.2. A polynucleotide encoding a protein comprising a modified version of STAT3, e.g., SEQ ID NO: 232), whose sequence comprises one or more mutations selected from F174S, H410R, S614R, E616K, G618R, Y640F, N6471, E652K, K658Y, K658R, K658N, K658M, K658R, K658H, K658N, D661Y and D661V is an embodiment of the invention. Exemplary mutated STAT3 proteins include SEQ ID NOs 246-250. Preferred embodiments comprise a polynucleotide comprising a nucleic acid encoding an STAT3 protein comprising an activating mutation, wherein the nucleic acid is operably linked to a heterologous promoter. Exemplary heterologous promoters that may be operably linked to the nucleic acid encoding an activating mutant of STAT3 include an EF1 promoter, a PGK promoter, a GAPDH promoter, an EEF2 promoter, a ubiquitin promoter, an SV40 promoter or an HSVTK promoter, for example a sequence selected from SEQ ID NOs 94-154. Preferred embodiments comprise a polynucleotide comprising a nucleic acid encoding an activating mutant of STAT3, wherein the nucleic acid is operably linked to a heterologous polyadenylation signal, for example a polyadenylation signal from a virus that infects mammalian cells, a mammalian EF1 polyadenylation signal, a mammalian growth hormone polyadenylation signal or a mammalian globin polyadenylation signal, for example a sequence selected from SEQ ID NOs 160-217. Preferred embodiments comprise a polynucleotide comprising a gene encoding an activating mutant of STAT3, wherein the polynucleotide is part of a piggyBac-like transposon which further comprises sequences with SEQ ID NOs: 6 and 7, or sequences with SEQ ID NOs: 14 and 15, or sequences with SEQ ID NOs: 18 and 19, or sequences with SEQ ID NOs:20 and 21. Preferred embodiments comprise a polynucleotide comprising a gene encoding an activating mutant of STAT3, wherein the polynucleotide is part of a Mariner transposon such as a Sleeping Beauty transposon which further comprises a sequence that is 90% identical to SEQ ID NO: 24 and a sequence that is 90% identical to SEQ ID NO: 25. Preferred embodiments comprise a polynucleotide comprising a gene encoding an activating mutant of STAT3, wherein the polynucleotide is part of an hAT transposon such as a TcBuster transposon which further comprises a sequence that is 90% identical to SEQ ID NO: 397 and a sequence that is 90% identical to SEQ ID NO: 398. The transposon comprising the polynucleotide encoding the activating mutant of STAT3 may be introduced into the immune cell together with a corresponding transposase or a polynucleotide encoding a corresponding transposase. Preferred embodiments comprise a polynucleotide comprising a gene encoding an activating mutant of STAT3, wherein the polynucleotide is part of a lentiviral vector. The lentiviral vector comprising the polynucleotide encoding the mutated STAT3 protein may be packaged and used to infect the immune cell. The immune cell is preferably a T-cell.

One aspect of the present invention is an immune cell whose genome comprises a heterologous polynucleotide comprising a gene encoding a STAT3 protein with an activating mutation. In some embodiments the heterologous polynucleotide comprises a lentiviral vector, or a piggyBac-like transposon, or a Mariner transposon such as a Sleeping Beauty transposon, or an hAT transposon such as a TcBuster transposon. In some embodiments, the immune cell genome comprises 3 copies of the STAT3 gene: two endogenous copies and one heterologous mutant copy.

### CD28

The CD28 (Cluster of Differentiation 28) gene is often found mutated in peripheral T-cell lymphomas. The most common activating mutations are D124E, D124V, T1951 and T195P. As described in Sections 6.2.1.2 and 6.2.1.3, we have demonstrated that a heterologous polynucleotide encoding an activating mutant of a CD28 protein may be introduced into an immune cell to enhance its survival or its proliferation, and to reduce restimulation-induced cell death; a gene encoding an activating mutant of CD28 is an immune cell survival-enhancing gene and an immune cell proliferation-enhancing gene as described in Section 6.2. A polynucleotide encoding a protein comprising a modified version of CD28 (e.g., SEQ ID NO: 233), whose sequence comprises one or more mutations selected from D124E, D124V, T1951 and T195P is an embodiment of the invention. An exemplary mutated CD28 protein is given as SEQ ID NO: 251. The mutated CD28 may further comprise replacement of the secretion signal in the first 18 amino acids of SEQ ID NO: 233 with another functionally active secretion signal. Preferred embodiments comprise a polynucleotide comprising a nucleic acid encoding an activating mutant of CD28, wherein the nucleic acid is operably linked to a heterologous promoter. Exemplary heterologous promoters that may be operably linked to the nucleic acid encoding mutated CD28 include an EF1 promoter, a PGK promoter, a GAPDH promoter, an EEF2 promoter, a ubiquitin promoter, an SV40 promoter or an HSVTK promoter, for example a sequence selected from SEQ ID NOs 94-154. Preferred embodiments comprise a polynucleotide comprising a nucleic acid encoding an activating mutant of CD28, wherein the nucleic acid is operably linked to a heterologous polyadenylation signal, for example a polyadenylation signal from a virus that infects mammalian cells, a mammalian EF1 polyadenylation signal, a mammalian growth hormone polyadenylation signal or a mammalian globin polyadenylation signal, for example a sequence selected from SEQ ID NOs 160-217. Preferred embodiments comprise a polynucleotide comprising a gene encoding an activating mutant of CD28, wherein the polynucleotide is part of a piggyBac-like transposon which further comprises sequences with SEQ ID NOs: 6 and 7, or sequences with SEQ ID NOs: 14 and 15, or sequences with SEQ ID NOs: 18 and 19, or sequences with SEQ ID NOs: 20 and 21. Preferred embodiments comprise a polynucleotide comprising a gene encoding an activating mutant of CD28, wherein the polynucleotide is part of a Mariner transposon such as a Sleeping Beauty transposon which further comprises a sequence that is 90% identical to SEQ ID NO: 24 and a sequence that is 90% identical to SEQ ID NO: 25. Preferred embodiments comprise a polynucleotide comprising a gene encoding an activating mutant of CD28, wherein the polynucleotide is part of an hAT transposon such as a TcBuster transposon which further comprises a sequence that is 90% identical to SEQ ID NO: 397 and a sequence that is 90% identical to SEQ ID NO: 398. The transposon comprising the polynucleotide encoding the activating mutant of CD28 may be introduced into the immune cell together with a corresponding transposase or a polynucleotide encoding a corresponding transposase. Preferred embodiments comprise a polynucleotide comprising a gene encoding an activating mutant of CD28, wherein the polynucleotide is part of a lentiviral vector. The lentiviral vector comprising the polynucleotide encoding the activating mutant of CD28 may be packaged and used to infect the immune cell. The immune cell is preferably a T-cell.

One aspect of the present invention is an immune cell whose genome comprises a heterologous polynucleotide comprising a gene encoding a CD28 protein with an activating mutation. In some embodiments the heterologous polynucleotide comprises a lentiviral vector, or a piggyBac-like transposon, or a Mariner transposon such as a Sleeping Beauty transposon, or an hAT transposon such as a TcBuster transposon. In some embodiments, the immune cell genome comprises 3 copies of the CD28 gene: two endogenous copies and one heterologous mutant copy.

### RhoA

The RhoA small GTPase is frequently mutated in peripheral T-cell lymphomas. The most common lymphoma-associated mutations are G17V and K18N. An activating mutant of a RhoA protein may be introduced into an immune cell to enhance its survival or its proliferation; a gene encoding an activating mutant of RhoA is an immune cell survival-enhancing gene and an immune cell proliferation-enhancing gene as described in Section 6.2. A polynucleotide encoding a protein comprising a modified version of RhoA, e.g., SEQ ID NO: 234, whose sequence comprises a mutation selected from G17V and K18N or a combination thereof is an embodiment of the invention. Exemplary mutated RhoA proteins are given as SEQ ID NOs: 252 and 253. Preferred embodiments comprise a polynucleotide comprising a nucleic acid encoding a mutated RhoA protein, wherein the nucleic acid is operably linked to a heterologous promoter. Exemplary heterologous promoters that may be operably linked to the gene encoding mutated RhoA include an EF1 promoter, a PGK promoter, a GAPDH promoter, an EEF2 promoter, a ubiquitin promoter, an SV40 promoter or an HSVTK promoter, for example a sequence selected from SEQ ID NOs 94-154. Preferred embodiments comprise a polynucleotide comprising a nucleic acid encoding a mutated RhoA protein, wherein the nucleic acid is operably linked to a heterologous polyadenylation signal, for example a polyadenylation signal from a virus that infects mammalian cells, a mammalian EF1 polyadenylation signal, a mammalian growth hormone polyadenylation signal or a mammalian globin polyadenylation signal, for example a sequence selected from SEQ ID NOs 160-217. Preferred embodiments comprise a polynucleotide comprising a gene encoding a mutated RhoA protein, wherein the polynucleotide is part of a piggyBac-like transposon which further comprises sequences with SEQ ID NOs: 6 and 7, or sequences with SEQ ID NOs: 14 and 15, or sequences with SEQ ID NOs: 18 and 19, or sequences with SEQ ID NOs: 20 and 21. Preferred embodiments comprise a polynucleotide comprising a gene encoding a mutated RhoA protein, wherein the polynucleotide is part of a Mariner transposon such as a Sleeping Beauty transposon which further comprises a sequence that is 90% identical to SEQ ID NO: 24 and a sequence that is 90% identical to SEQ ID NO: 25. Preferred embodiments comprise a polynucleotide comprising a gene encoding a mutated RhoA protein, wherein the polynucleotide is part of an hAT transposon such as a TcBuster transposon which further comprises a sequence that is 90% identical to SEQ ID NO: 397 and a sequence that is 90% identical to SEQ ID NO: 398. The transposon comprising the polynucleotide encoding the mutated RhoA protein may introduced into the immune cell together with a polynucleotide encoding a corresponding transposase. Preferred embodiments comprise a polynucleotide comprising a gene encoding a mutated RhoA protein, wherein the polynucleotide is part of a lentiviral vector. The lentiviral vector comprising the polynucleotide encoding the mutated RhoA protein may be packaged and used to infect the immune cell. The immune cell is preferably a T-cell.

One aspect of the present invention is an immune cell whose genome comprises a heterologous polynucleotide comprising a gene encoding a RhoA protein with an activating mutation. In some embodiments the heterologous polynucleotide comprises a lentiviral vector, or a piggyBac-like transposon, or a Mariner transposon such as a Sleeping Beauty transposon, or an hAT transposon such as a TcBuster transposon. In some embodiments, the immune cell genome comprises 3 copies of the RhoA gene: two endogenous copies and one heterologous mutant copy.

### Phospholipase C, Gamma 1

Activating phospholipase C gamma (PLCG) mutations have been associated with cutaneous T-cell lymphomas. The most common lymphoma-associated activating mutations are S345F, S520F and R707Q. As described in Section 6.2.1.5, we have demonstrated that a heterologous polynucleotide encoding an activating mutant of a PLCG protein may be introduced into an immune cell to enhance its survival or its proliferation; a gene encoding an activating mutant of PLCG is an immune cell survival-enhancing gene and an immune cell proliferation-enhancing gene as described in Section 6.2. A polynucleotide encoding a protein comprising a modified version of PLCG, e.g., SEQ ID NO: 235, whose sequence comprises one or more mutations selected from S345F, S520F and R707Q is an embodiment of the invention. An exemplary mutated PLCG protein is given as SEQ ID NO: 254. Preferred embodiments comprise a polynucleotide comprising a gene encoding an activating mutant of PLCG, wherein the nucleic acid is operably linked to a heterologous promoter. Exemplary heterologous promoters that may be operably linked to the gene encoding mutated PLCG include an EF1 promoter, a PGK promoter, a GAPDH promoter, an EEF2 promoter, a ubiquitin promoter, an SV40 promoter or an HSVTK promoter, for example a sequence selected from SEQ ID NOs 94-154. Preferred embodiments comprise a polynucleotide comprising a nucleic acid encoding an activating mutant of PLCG, wherein the nucleic acid is operably linked to a heterologous polyadenylation signal, for example a polyadenylation signal from a virus that infects mammalian cells, a mammalian EF1 polyadenylation signal, a mammalian growth hormone polyadenylation signal or a mammalian globin polyadenylation signal, for example a sequence selected from SEQ ID NOs 160-217. Preferred embodiments comprise a polynucleotide comprising a gene encoding an activating mutant of PLCG, wherein the polynucleotide is part of a piggyBac-like transposon which further comprises sequences with SEQ ID NOs: 6 and 7, or sequences with SEQ ID NOs: 14 and 15, or sequences with SEQ ID NOs: 18 and 19, or sequences with SEQ ID NOs: 20 and 21. Preferred embodiments comprise a polynucleotide comprising a gene encoding an activating mutant of PLCG, wherein the polynucleotide is part of a Mariner transposon such as a Sleeping Beauty transposon which further comprises a sequence that is 90% identical to SEQ ID NO: 24 and a sequence that is 90% identical to SEQ ID NO: 25. Preferred embodiments comprise a polynucleotide comprising a gene encoding a mutated PLCG protein, wherein the polynucleotide is part of an hAT transposon such as a TcBuster transposon which further comprises a sequence that is 90% identical to SEQ ID NO: 397 and a sequence that is 90% identical to SEQ ID NO: 398. The transposon comprising the polynucleotide encoding the mutated PLCG protein may be introduced into the immune cell together with a corresponding transposase or a polynucleotide encoding a corresponding transposase. Preferred embodiments comprise a polynucleotide comprising a gene encoding a mutated PLCG protein, wherein the polynucleotide is part of a lentiviral vector. The lentiviral vector comprising the polynucleotide encoding the mutated PLCG protein may be packaged and used to infect the immune cell. The immune cell is preferably a T-cell.

One aspect of the present invention is an immune cell whose genome comprises a heterologous polynucleotide comprising a gene encoding a PLCG protein with an activating mutation. In some embodiments the heterologous polynucleotide comprises a lentiviral vector, or a piggyBac-like transposon, or a Mariner transposon such as a Sleeping Beauty transposon, or an hAT transposon such as a TcBuster transposon. In some embodiments, the immune cell genome comprises 3 copies of the PLCG gene: two endogenous copies and one heterologous mutant copy.

### STAT5B

The gene encoding STAT5B (signal transducer and activator of transcription 5B) is sometimes found to be mutated in T-cell leukemias. The most common leukemia-associated activating mutation is N642H in the SH2 domain. Other STAT5B activating mutations associated with T-cell cancers include SH2 domain mutations T648S, S652Y and Y665F, as well as P267A outside the SH2 domain. A heterologous polynucleotide encoding an activating mutant of a STAT5B protein may be introduced into an immune cell to enhance its survival or its proliferation; a gene encoding an activating mutant of STAT5B is an immune cell survival-enhancing gene and an immune cell proliferation-enhancing gene as described in Section 6.2. A polynucleotide encoding a protein comprising a modified version of STAT5B (e.g., SEQ ID NO: 236), whose sequence comprises one or more mutations selected from N642H, T648S, S652Y, Y665F and P267A is an embodiment of the invention. An exemplary mutated STAT5B protein is given as SEQ ID NO: 255. Preferred embodiments comprise a polynucleotide comprising a nucleic acid encoding a mutated STAT5B protein, wherein the nucleic acid is operably linked to a heterologous promoter. Exemplary heterologous promoters that may be operably linked to the gene encoding mutated STAT5B include an EF1 promoter, a PGK promoter, a GAPDH promoter, an EEF2 promoter, a ubiquitin promoter, an SV40 promoter or an HSVTK promoter, for example a sequence selected from SEQ ID NOs 94-154. Preferred embodiments comprise a polynucleotide comprising a gene encoding a mutated STAT5B protein, wherein the gene is operably linked to a heterologous polyadenylation signal, for example a polyadenylation signal from a virus that infects mammalian cells, a mammalian EF1 polyadenylation signal, a mammalian growth hormone polyadenylation signal or a mammalian globin polyadenylation signal, for example a sequence selected from SEQ ID NOs 160-217. Preferred embodiments comprise a polynucleotide comprising a gene encoding a mutated STAT5B protein, wherein the polynucleotide is part of a piggyBac-like transposon which further comprises sequences with SEQ ID NOs: 6 and 7, or sequences with SEQ ID NOs: 14 and 15, or sequences with SEQ ID NOs: 18 and 19, or sequences with SEQ ID NOs: 20 and 21. Preferred embodiments comprise a polynucleotide comprising a gene encoding an activating mutant of STAT5B, wherein the polynucleotide is part of a Mariner transposon such as a Sleeping Beauty transposon which further comprises a sequence that is 90% identical to SEQ ID NO: 24 and a sequence that is 90% identical to SEQ ID NO: 25. Preferred embodiments comprise a polynucleotide comprising a gene encoding a mutated STAT5B protein, wherein the polynucleotide is part of an hAT transposon such as a TcBuster transposon which further comprises a sequence that is 90% identical to SEQ ID NO: 397 and a sequence that is 90% identical to SEQ ID NO: 398. The transposon comprising the polynucleotide encoding the mutated STAT5B protein may be introduced into the immune cell together with a corresponding transposase or a polynucleotide encoding a corresponding transposase. Preferred embodiments comprise a polynucleotide comprising a gene encoding a mutated STAT5B protein, wherein the polynucleotide is part of a lentiviral vector. The lentiviral vector comprising the polynucleotide encoding the mutated STAT5B protein may be packaged and used to infect the immune cell. The immune cell is preferably a T-cell.

One aspect of the present invention is an immune cell whose genome comprises a heterologous polynucleotide comprising a gene encoding a STAT5B protein with an activating mutation. In some embodiments the heterologous polynucleotide comprises a lentiviral vector, or a piggyBac-like transposon, or a Mariner transposon such as a Sleeping Beauty transposon, or an hAT transposon such as a TcBuster transposon. In some embodiments, the immune cell genome comprises 3 copies of the STAT5B gene: two endogenous copies and one heterologous mutant copy.

### Survivin

The gene encoding Survivin (a member of the Inhibitor of Apoptosis family of proteins) is sometimes found to be upregulated in T-cell leukemias. As described in Section 6.2.1.3, we have demonstrated that a heterologous polynucleotide encoding a Survivin gene operably linked to a heterologous promoter may be introduced into an immune cell to enhance its survival or its proliferation, and to reduce restimulation-induced cell death; a Survivin gene operably linked to a heterologous promoter is an immune cell survival-enhancing gene and an immune cell proliferation-enhancing gene as described in Section 6.2. A polynucleotide encoding a protein comprising SEQ ID NO: 237 operably linked to a heterologous promoter is an embodiment of the invention. Exemplary heterologous promoters that may be operably linked to the gene encoding Survivin include an EF1 promoter, a PGK promoter, a GAPDH promoter, an EEF2 promoter, a ubiquitin promoter, an SV40 promoter or an HSVTK promoter, for example a sequence selected from SEQ ID NOs 94-154. Preferred embodiments comprise a polynucleotide comprising a gene encoding Survivin, wherein the gene is operably linked to a heterologous polyadenylation signal, for example a polyadenylation signal from a virus that infects mammalian cells, a mammalian EF1 polyadenylation signal, a mammalian growth hormone polyadenylation signal or a mammalian globin polyadenylation signal, for example a sequence selected from SEQ ID NOs 160-217. Preferred embodiments comprise a polynucleotide comprising a gene encoding Survivin, wherein the polynucleotide is part of a piggyBac-like transposon which further comprises sequences with SEQ ID NOs: 6 and 7, or sequences with SEQ ID NOs: 14 and 15, or sequences with SEQ ID NOs: 18 and 19, or sequences with SEQ ID NOs: 20 and 21. Preferred embodiments comprise a polynucleotide comprising a gene encoding Survivin wherein the polynucleotide is part of a Mariner transposon such as a Sleeping Beauty transposon which further comprises a sequence that is 90% identical to SEQ ID NO: 24 and a sequence that is 90% identical to SEQ ID NO: 25. Preferred embodiments comprise a polynucleotide comprising a gene encoding a gene encoding Survivin, wherein the polynucleotide is part of an hAT transposon such as a TcBuster transposon which further comprises a sequence that is 90% identical to SEQ ID NO: 397 and a sequence that is 90% identical to SEQ ID NO: 398. The transposon comprising the polynucleotide encoding Survivin may be introduced into the immune cell together with a corresponding transposase or a polynucleotide encoding a corresponding transposase. Preferred embodiments comprise a polynucleotide comprising a gene encoding Survivin, wherein the polynucleotide is part of a lentiviral vector. The lentiviral vector comprising the polynucleotide encoding Survivin may be packaged and used to infect the immune cell. The immune cell is preferably a T-cell or a B-cell.

One aspect of the present invention is an immune cell whose genome comprises a heterologous polynucleotide comprising a gene encoding Survivin and further comprising a lentiviral vector, or a piggyBac-like transposon, or a Mariner transposon such as a Sleeping Beauty transposon, or an hAT transposon such as a TcBuster transposon. In some embodiments, the immune cell genome comprises 3 copies of the Survivin gene: two endogenous copies and one heterologous copy operably linked to a heterologous promoter.

### Bel-XL

The gene encoding Bcl-XL (an anti-apoptotic protein) is sometimes found to be unregulated in B-cell lymphomas. As described in Section 6.2.1.5 and Section 6.2.1.6, we have demonstrated that a heterologous polynucleotide encoding a Bcl-XL gene operably linked to a heterologous promoter may be introduced into an immune cell to enhance its survival or its proliferation, and to reduce restimulation-induced cell death; a Bcl-XL gene operably linked to a heterologous promoter is an immune cell survival-enhancing gene and an immune cell proliferation-enhancing gene as described in Section 6.2. A polynucleotide encoding a protein comprising SEQ ID NO: 238 operably linked to a heterologous promoter is an embodiment of the invention. Exemplary heterologous promoters that may be operably linked to a nucleic acid encoding Bcl-XL include an EF1 promoter, a PGK promoter, a GAPDH promoter, an EEF2 promoter, a ubiquitin promoter, an SV40 promoter or an HSVTK promoter, for example a sequence selected from SEQ ID NOs 94-154. Preferred embodiments comprise a polynucleotide comprising a nucleic acid encoding Bcl-XL, wherein the nucleic acid is operably linked to a heterologous polyadenylation signal, for example a polyadenylation signal from a virus that infects mammalian cells, a mammalian EF1 polyadenylation signal, a mammalian growth hormone polyadenylation signal or a mammalian globin polyadenylation signal for example a sequence selected from SEQ ID NOs 160-217. Preferred embodiments comprise a polynucleotide comprising a gene encoding Bcl-XL, wherein the polynucleotide is part of a piggyBac-like transposon which further comprises sequences with SEQ ID NOs: 6 and 7, or sequences with SEQ ID NOs: 14 and 15, or sequences with SEQ ID NOs: 18 and 19, or sequences with SEQ ID NOs: 20 and 21. Preferred embodiments comprise a polynucleotide comprising a gene encoding Bcl-XL wherein the polynucleotide is part of a Mariner transposon such as a Sleeping Beauty transposon which further comprises a sequence that is 90% identical to SEQ ID NO: 24 and a sequence that is 90% identical to SEQ ID NO: 25. Preferred embodiments comprise a polynucleotide comprising a gene encoding a gene encoding Bcl-XL, wherein the polynucleotide is part of an hAT transposon such as a TcBuster transposon which further comprises a sequence that is 90% identical to SEQ ID NO: 397 and a sequence that is 90% identical to SEQ ID NO: 398. The transposon comprising the polynucleotide encoding Bcl-XL may be introduced into the immune cell together with a polynucleotide encoding a corresponding transposase. Preferred embodiments comprise a polynucleotide comprising a gene encoding Bcl-XL, wherein the polynucleotide is part of a lentiviral vector. The lentiviral vector comprising the polynucleotide encoding Bcl-XL may be packaged and used to infect the immune cell. The immune cell is preferably a T-cell or a B-cell.

One aspect of the present invention is an immune cell whose genome comprises a heterologous polynucleotide comprising a gene encoding Bcl-XL and further comprising a lentiviral vector, or a piggyBac-like transposon, or a Mariner transposon such as a Sleeping Beauty transposon, or an hAT transposon such as a TcBuster transposon. In some embodiments, the immune cell genome comprises 3 copies of the Bcl-XL gene: two endogenous copies and one heterologous copy operably linked to a heterologous promoter.

### CCND1

The gene encoding CCND1 (cyclin D1) is sometimes found to be mutated in leukemias. CCND1 mutations associated with cancers include E36G, E36Q, E36K, A39S, S41L, S41P, S41T, V42E, V42A, V42L, V42M, Y44S, Y44D, Y44C, Y44H, K46T, K46R, K46N, K46E, C47G, C47R, C47S, C47W, P199R, P199S, P199L, S201F, T2851, T285A, P286L, P286H, P286S, P286T and P286A. A heterologous polynucleotide encoding an activating mutant of a CCND1 protein may be introduced into an immune cell to enhance its survival or its proliferation; a gene encoding an activating mutant of CCND1 is an immune cell survival-enhancing gene and an immune cell proliferation-enhancing gene as described in Section 6.2. A polynucleotide encoding a protein comprising a modified version of CCND1 (e.g., SEQ ID NO: 239), whose sequence comprises one or more mutations selected from E36G, E36Q, E36K, A39S, S41L, S41P, S41T, V42E, V42A, V42L, V42M, Y44S, Y44D, Y44C, Y44H, K46T, K46R, K46N, K46E, C47G, C47R, C47S, C47W, P199R, P199S, P199L, S201F, T2851, T285A, P286L, P286H, P286S, P286T and P286A is an embodiment of the invention. An exemplary mutated CCND1 protein is given as SEQ ID NO: 256. Preferred embodiments comprise a polynucleotide comprising a nucleic acid encoding a mutated CCND1 protein, wherein the nucleic acid is operably linked to a heterologous promoter. Exemplary heterologous promoters that may be operably linked to the nucleic acid encoding mutated CCND1 include an EF1 promoter, a PGK promoter, a GAPDH promoter, an EEF2 promoter, a ubiquitin promoter, an SV40 promoter or an HSVTK promoter, for example a sequence selected from SEQ ID NOs 94-154. Preferred embodiments comprise a polynucleotide comprising a nucleic acid encoding a mutated CCND1 protein, wherein the nucleic acid is operably linked to a heterologous polyadenylation signal, for example a polyadenylation signal from a virus that infects mammalian cells, a mammalian EF1 polyadenylation signal, a mammalian growth hormone polyadenylation signal or a mammalian globin polyadenylation signal, for example a sequence selected from SEQ ID NOs 160-217. Preferred embodiments comprise a polynucleotide comprising a gene encoding a mutated CCND1 protein, wherein the polynucleotide is part of a piggyBac-like transposon which further comprises sequences with SEQ ID NOs: 6 and 7, or sequences with SEQ ID NOs: 14 and 15, or sequences with SEQ ID NOs: 18 and 19, or sequences with SEQ ID NOs: 20 and 21. Preferred embodiments comprise a polynucleotide comprising a gene encoding an activating mutant of CCND1, wherein the polynucleotide is part of a Mariner transposon such as a Sleeping Beauty transposon which further comprises a sequence that is 90% identical to SEQ ID NO: 24 and a sequence that is 90% identical to SEQ ID NO: 25. Preferred embodiments comprise a polynucleotide comprising a gene encoding a gene encoding an activating mutant of CCND1, wherein the polynucleotide is part of an hAT transposon such as a TcBuster transposon which further comprises a sequence that is 90% identical to SEQ ID NO: 397 and a sequence that is 90% identical to SEQ ID NO: 398. The transposon comprising the polynucleotide encoding the mutated CCND1 protein may introduced into the immune cell together with a polynucleotide encoding a corresponding transposase. Preferred embodiments comprise a polynucleotide comprising a gene encoding a mutated CCND1 protein, wherein the polynucleotide is part of a lentiviral vector. The lentiviral vector comprising the polynucleotide encoding the mutated CCND1 protein may be packaged and used to infect the immune cell. The immune cell is preferably a T-cell or a B-cell.

One aspect of the present invention is an immune cell whose genome comprises a heterologous polynucleotide comprising a gene encoding a CCND1 protein with an activating mutation. In some embodiments the heterologous polynucleotide comprises a lentiviral vector, or a piggyBac-like transposon, or a Mariner transposon such as a Sleeping Beauty transposon, or a hAT transposon such as a TcBuster transposon. In some embodiments, the immune cell genome comprises 3 copies of the CCND1 gene: two endogenous copies and one heterologous mutant copy.

### Bcl2

The gene encoding Bcl2 (an anti-apoptotic protein) is sometimes found to be upregulated in B-cell lymphomas. As described in Section 6.2.1.4, we have demonstrated that a heterologous polynucleotide encoding a Bcl2 gene operably linked to a heterologous promoter may be introduced into an immune cell to enhance its survival or its proliferation; a Bcl2 gene operably linked to a heterologous promoter is an immune cell survival-enhancing gene and an immune cell proliferation-enhancing gene as described in Section 6.2. A polynucleotide encoding a protein comprising SEQ ID NO: v270 or 272 operably linked to a heterologous promoter is an embodiment of the invention. Exemplary heterologous promoters that may be operably linked to a nucleic acid encoding Bcl2 include an EF1 promoter, a PGK promoter, a GAPDH promoter, an EEF2 promoter, a ubiquitin promoter, an SV40 promoter or an HSVTK promoter, for example a sequence selected from SEQ ID NOs 94-154. Preferred embodiments comprise a polynucleotide comprising a nucleic acid encoding Bcl2, wherein the nucleic acid is operably linked to a heterologous polyadenylation signal, for example a polyadenylation signal from a virus that infects mammalian cells, a mammalian EF1 polyadenylation signal, a mammalian growth hormone polyadenylation signal or a mammalian globin polyadenylation signal, for example a sequence selected from SEQ ID NOs 160-217. Preferred embodiments comprise a polynucleotide comprising a gene encoding Bcl2, wherein the polynucleotide is part of a piggyBac-like transposon which further comprises sequences with SEQ ID NOs: 6 and 7, or sequences with SEQ ID NOs: 14 and 15, or sequences with SEQ ID NOs: 18 and 19, or sequences with SEQ ID NOs: 20 and 21. Preferred embodiments comprise a polynucleotide comprising a gene encoding Bcl2 wherein the polynucleotide is part of a Mariner transposon such as a Sleeping Beauty transposon which further comprises a sequence that is 90% identical to SEQ ID NO: 24 and a sequence that is 90% identical to SEQ ID NO: 25. Preferred embodiments comprise a polynucleotide comprising a gene encoding a gene encoding Bcl2, wherein the polynucleotide is part of an hAT transposon such as a TcBuster transposon which further comprises a sequence that is 90% identical to SEQ ID NO: 397 and a sequence that is 90% identical to SEQ ID NO: 398. The transposon comprising the polynucleotide encoding Bcl2 may be introduced into the immune cell together with a polynucleotide encoding a corresponding transposase. Preferred embodiments comprise a polynucleotide comprising a gene encoding Bcl2, wherein the polynucleotide is part of a lentiviral vector. The lentiviral vector comprising the polynucleotide encoding Bcl2 may be packaged and used to infect the immune cell. The immune cell is preferably a T-cell or a B-cell.

One aspect of the present invention is an immune cell whose genome comprises a heterologous polynucleotide comprising a gene encoding Bcl2 and further comprising a lentiviral vector, or a piggyBac-like transposon, or a Mariner transposon such as a Sleeping Beauty transposon, or an hAT transposon such as a TcBuster transposon. In some embodiments, the immune cell genome comprises 3 copies of the Bcl2 gene: two endogenous copies and one heterologous copy operably linked to a heterologous promoter.

### Bcl6

The gene encoding Bcl6 (an anti-apoptotic protein) is sometimes found to be unregulated in B-cell lymphomas. As described in Section 6.2.1.4, we have demonstrated that a heterologous polynucleotide encoding a Bcl6 gene operably linked to a heterologous promoter may be introduced into an immune cell to enhance its survival or its proliferation; a Bcl6 gene operably linked to a heterologous promoter is an immune cell survival-enhancing gene and an immune cell proliferation-enhancing gene as described in Section 6.2. A polynucleotide encoding a protein comprising SEQ ID NO: 271 or 272 operably linked to a heterologous promoter is an embodiment of the invention. Exemplary heterologous promoters that may be operably linked to a nucleic acid encoding Bcl6 include an EF1 promoter, a PGK promoter, a GAPDH promoter, an EEF2 promoter, a ubiquitin promoter, an SV40 promoter or an HSVTK promoter, for example a sequence selected from SEQ ID NOs 94-154. Preferred embodiments comprise a polynucleotide comprising a nucleic acid encoding Bcl6, wherein the nucleic acid is operably linked to a heterologous polyadenylation signal, for example a polyadenylation signal from a virus that infects mammalian cells, a mammalian EF1 polyadenylation signal, a mammalian growth hormone polyadenylation signal or a mammalian globin polyadenylation signal for example a sequence selected from SEQ ID NOs 160-217. Preferred embodiments comprise a polynucleotide comprising a gene encoding Bcl6, wherein the polynucleotide is part of a piggyBac-like transposon which further comprises sequences with SEQ ID NOs: 6 and 7, or sequences with SEQ ID NOs: 14 and 15, or sequences with SEQ ID NOs: 18 and 19, or sequences with SEQ ID NOs: 20 and 21. Preferred embodiments comprise a polynucleotide comprising a gene encoding Bcl6 wherein the polynucleotide is part of a Mariner transposon such as a Sleeping Beauty transposon which further comprises a sequence that is 90% identical to SEQ ID NO: 24 and a sequence that is 90% identical to SEQ ID NO: 25. Preferred embodiments comprise a polynucleotide comprising a gene encoding a gene encoding Bcl6, wherein the polynucleotide is part of an hAT transposon such as a TcBuster transposon which further comprises a sequence that is 90% identical to SEQ ID NO: 397 and a sequence that is 90% identical to SEQ ID NO: 398. The transposon comprising the polynucleotide encoding Bcl6 may be introduced into the immune cell together with a corresponding transposase or a polynucleotide encoding a corresponding transposase. Preferred embodiments comprise a polynucleotide comprising a gene encoding Bcl6, wherein the polynucleotide is part of a lentiviral vector. The lentiviral vector comprising the polynucleotide encoding Bcl6 may be packaged and used to infect the immune cell. The immune cell is preferably a T-cell or a B-cell.

One aspect of the present invention is an immune cell whose genome comprises a heterologous polynucleotide comprising a gene encoding Bcl6 and further comprising a lentiviral vector or a piggyBac-like transposon. In some embodiments, the immune cell genome comprises 3 copies of the Bcl6 gene: two endogenous copies and one heterologous copy operably linked to a heterologous promoter.

### Enhanced Signalling Receptors

Immune cells such as T-cells express membrane proteins that comprise an extracellular domain that binds to naturally occurring and synthetic ligands, a transmembrane domain and an intracellular domain that interacts with intracellular signaling pathways. We have designed, synthesized and tested a set of chimeric receptors, which we call Enhanced Signaling Receptors (ESRs), which comprise an extracellular domain derived from a first protein, a transmembrane domain and an intracellular domain derived from a receptor that transmits a stimulatory or co-stimulatory signal to an immune cell. Unlike chimeric antigen receptors, however, ESRs do not comprise a sequence comprising the intracellular portion of the CD3 zeta chain. One function of ESRs is to enhance immune cell survival. Another function of ESRs is to counteract the engagement of T-cell inhibitory pathways, for example by tumor cells acting on inhibitory receptors (Tay et al, 2017. Immunotherapy 9, 1339-1349). For ESRs to function effectively, they must be expressed at high enough levels to compete with the natural inhibitory receptor for the inhibitory ligand being presented within the tumor microenvironment.

In one embodiment, the extracellular domain of the ESR may be derived from the extracellular ligand binding domain of a receptor that naturally transmits an inhibitory signal to an immune cell: in this case an ESR receives what is normally interpreted as an inhibitory signal and transduces it as stimulatory signal. For example the extracellular domain of an ESR may comprise a sequence derived from the extracellular domain of a protein selected from TNFRSF1A, TNFRSF3 (LTRβ), TNFRSF6 (Fas), TNFRSF8 (CD30), TNFRSF10A (DR4), TNFRSF10B (DR5), TNFRSF19 (TROY), TNFRSF21 (DR6) and CTLA4; preferably the extracellular domain is derived from a human protein. In some embodiments of the invention the extracellular domain of an ESR comprises a polypeptide whose sequence is at least 90% identical, or at least 95% identical, or at least 96% identical or at least 97% identical to or at least 98% identical to or at least 99% or 100% identical to a sequence selected from SEQ ID NOs: 322-330.

In another embodiment, the extracellular domain of the ESR may be derived from a protein that binds to a protein expressed on the surface of an immune cell, preferably a protein whose normal function is to stimulate immune function: in this case an ESR transmits a stimulatory signal to another immune cell and transduces a stimulatory signal to the immune cell in which it is expressed. For example the ESR extracellular domain may comprise the variable domain of an antibody, a single chain antibody, a single domain antibody, a nanobody, a V_{H}H fragment or a V_{NAR} fragment that binds to the extracellular domain of a protein selected from TNFRSF4 (OX40), TNFRSF5 (CD40), TNFRSF7 (CD27), TNFRSF9 (4-1BB), TNFRSF11A (RANK), TNFRSF13B (TACI), TNFRSF13C (BAFF-R), TNFRSF14 (HVEM), TNFRSF17 (CD269), TNFRSF18 (GITR), CD28, CD28H (TMIGD2), Inducible T-cell Costimulator (ICOS/CD278), DNAX Accessory Molecule-1 (DNAM-1/CD226), Signaling Lymphocytic Activation Molecule (SLAM/CD150), T-cell Immunoglobulin and Mucin domain (TIM-1/HAVcr-1), interferon receptor alpha chain (IFNAR1), interferon receptor beta chain IFNAR2), interleukin-2 receptor beta subunit (IL2RB) and interleukin-2 receptor gamma subunit (IL2RG). An exemplary single chain anti-CD28 antibody is TGN1412, with sequence SEQ ID NO: 340.

In another embodiment, the extracellular domain of the ESR may be derived from a ligand that binds to a receptor expressed on the surface of an immune cell, preferably a receptor whose normal function is to transduce a stimulatory or co-stimulatory signal in the immune cell: in this case an ESR transmits a stimulatory signal to another immune cell and transduces a stimulatory signal to the immune cell in which it is expressed. For example, the ESR extracellular domain may comprise a sequence derived from the extracellular domain of a protein selected from TNFSF4 (OX40 ligand), TNFSF5 (CD40 ligand), TNFSF9 (4-1BB ligand), TNFSF11 (RANKL), TNFSF14 (HVEM ligand), TNFSF13B, CD80, CD86 and ICOS ligand; preferably the extracellular domain is derived from a human protein. In some embodiments of the invention the extracellular domain of an ESR comprises a polypeptide whose sequence is at least 90% identical, or at least 95% identical, or at least 96% identical or at least 97% identical to or at least 98% identical to or at least 99% identical to a sequence selected from SEQ ID NOs: 331-339.

In some embodiments of the invention, an Enhanced Signaling Receptor comprises a sequence derived from the intracellular domain of a member of the Tumor Necrosis Factor Receptor Superfamily (TNFRSF) or another immune cell receptor that normally transmits a stimulatory signal to an immune cell; in some embodiments of the invention the ESR comprises a sequence derived from the intracellular domain of a protein selected from TNFRSF4 (OX40), TNFRSF5 (CD40), TNFRSF7 (CD27), TNFRSF9 (4-1BB), TNFRSF (RANK), TNFRSF13B (TACI), TNFRSF13C (BAFF-R), TNFRSF14 (HVEM), TNFRSF17 (CD269), TNFRSF18 (GITR), CD28, CD28H (TMIGD2), Inducible T-cell Costimulator (ICOS/CD278), DNAX Accessory Molecule-1 (DNAM-1/CD226), Signaling Lymphocytic Activation Molecule (SLAM/CD150), T-cell Immunoglobulin and Mucin domain (TIM-1/HAVcr-1), interferon receptor alpha chain (IFNAR1), interferon receptor beta chain IFNAR2), interleukin-2 receptor beta subunit (IL2RB), interleukin-2 receptor gamma subunit (IL2RG), Tumor Necrosis Factor Superfamily 14 (TNFSF14/LIGHT), Natural Killer Group 2 member D (NKG2D/CD314) and CD40 ligand (CD40L); preferably the intracellular domain is of a human protein. In some embodiments of the invention the ESR comprises a polypeptide whose sequences is at least 90% identical, or at least 95% identical, or at least 96% identical or at least 97% identical to or at least 98% identical to or at least 99% or 100% identical to a sequence selected from SEQ ID NOs: 341-364.

In some embodiments of the invention, an Enhanced Signaling Receptor comprises a sequence derived from the transmembrane domain of a member of the Tumor Necrosis Factor Receptor Superfamily (TNFRSF) or another immune cell receptor that normally transmits an inhibitory or stimulatory signal to an immune cell; in some embodiments of the invention the ESR comprises a sequence derived from the transmembrane domain of a protein selected from TNFRSF1A, TNFRSF1B, TNFRSF3 (LTRβ), TNFRSF6 (Fas), TNFRSF8 (CD30), TNFRSF10A (DR4), TNFRSF10B (DR5), TNFRSF19 (TROY), TNFRSF21 (DR6), CTLA4, TNFRSF4 (OX40), TNFRSF5 (CD40), TNFRSF7 (CD27), TNFRSF9 (4-1BB), TNFRSF11A (RANK), TNFRSF13B (TACI), TNFRSF13C (BAFF-R), TNFRSF14 (HVEM), TNFRSF17 (CD269), TNFRSF18 (GITR), CD28, CD28H (TMIGD2), Inducible T-cell Costimulator (ICOS/CD278), DNAX Accessory Molecule-1 (DNAM-1/CD226), Signaling Lymphocytic Activation Molecule (SLAM/CD150), T-cell Immunoglobulin and Mucin domain (TIM-1/HAVcr-1), interferon receptor alpha chain (IFNAR1), interferon receptor beta chain IFNAR2), interleukin-2 receptor beta subunit (IL2RB), interleukin-2 receptor gamma subunit (IL2RG), Tumor Necrosis Factor Superfamily 14 (TNFSF14/LIGHT), Natural Killer Group 2 member D (NKG2D/CD314) and CD40 ligand (CD40L); preferably the transmembrane domain is of a human protein; in some embodiments of the invention the ESR comprises a polypeptide whose sequences is at least 90% identical, or at least 95% identical, or at least 96% identical or at least 97% identical to or at least 98% identical to or at least 99% or 100% identical to a sequence selected from SEQ ID NOs: 365-396.

In some embodiments of the invention, an Enhanced Signaling Receptor comprises a sequence at least 90% identical, or at least 95% identical, or at least 96% identical or at least 97% identical to or at least 98% identical to or at least 99% or 100% identical to a sequence selected from SEQ ID NOs: 274-318. These sequences comprise an N-terminal secretion signal (for example MLGIWTLLPLVLTSVARLSSKSVNA, MEQRPRGCAAVAAALLLVLLGARAQG, MGLSTVPDLLLPLVLLELLVGIYPSGVIG, MGTSPSSSTALASCSRIARRATATMIAGSLLLLGFLSTTTA, MEQRGQNAPAASGARKRHGPGPREARGARPGPRVPKTLVLVVAAVLLLVSAES and MAVMAPRTLVLLLSGALALTQTWA are signal sequences for these ESRs). Signal sequences function to translocate the ESR into the membrane. The signal sequence of an ESR is removed by a signal peptidase and does not form a part of the final receptor, so any functional secretion signal may be replaced by another functional secretion signal without altering the activity of the ESR. Such replacements are expressly contemplated. An Enhanced Signaling Receptor comprises a sequence at least 90% identical, or at least 95% identical, or at least 96% identical or at least 97% identical to or at least 98% identical to or at least 99% or 100% identical to the non-signal sequence portion of a sequence selected from SEQ ID NOs: 274-318.

In some embodiments of the invention, a gene encoding an ESR is expressed in an immune cell, for example a T-cell, and increases the survival or the proliferation of the immune cell, or the ability of a T-cell to kill a cell within a tumor microenvironment. An immune cell whose genome comprises a gene encoding an ESR that increases the survival or the proliferation of the immune cell or the ability of a T-cell to kill a cell within a tumor microenvironment is an aspect of the invention.

Preferred embodiments comprise a polynucleotide comprising a gene encoding an ESR, wherein the gene is operably linked to a heterologous promoter. Exemplary heterologous promoters that may be operably linked to the gene encoding an ESR include an EF1 promoter, a PGK promoter, a GAPDH promoter, an EEF2 promoter, a ubiquitin promoter, an SV40 promoter or an HSVTK promoter, for example a sequence selected from SEQ ID NOs 94-154. Preferred embodiments comprise a polynucleotide comprising a gene encoding an ESR, wherein the gene is operably linked to a heterologous polyadenylation signal, for example a polyadenylation signal from a virus that infects mammalian cells, a mammalian EF1 polyadenylation signal, a mammalian growth hormone polyadenylation signal or a mammalian globin polyadenylation signal, for example a sequence selected from SEQ ID NOs 160-217. Some embodiments comprise a polynucleotide comprising a gene encoding an ESR, wherein the polynucleotide is part of a lentiviral vector. The lentiviral vector comprising the polynucleotide encoding the ESR may be packaged and used to infect the immune cell. More preferably the ESR is encoded on a gene transfer polynucleotide that is part of a piggyBac-like transposon, for example a polynucleotide which further comprises sequences with SEQ ID NOs: 6 and 7, or sequences with SEQ ID NOs: 14 and 15, or sequences with SEQ ID NOs: 18 and 19, or sequences with SEQ ID NOs: 20 and 21. The ESR may be encoded on a gene transfer polynucleotide that is part of a Mariner transposon such as a Sleeping Beauty transposon which further comprises a sequence that is 90% identical to SEQ ID NO: 24 and a sequence that is 90% identical to SEQ ID NO: 25. The ESR may be encoded on a gene transfer polynucleotide that is part of an hAT transposon such as a TcBuster transposon which further comprises a sequence that is 90% identical to SEQ ID NO: 397 and a sequence that is 90% identical to SEQ ID NO: 398. The gene transfer polynucleotide comprising the transposon plus the polynucleotide encoding the ESR may further comprise a gene encoding a chimeric antigen receptor. The gene transfer polynucleotide may be introduced into the immune cell together with a corresponding transposase, which may be provided as a polynucleotide encoding the transposase. The immune cell is preferably a T-cell.

One aspect of the present invention is an immune cell whose genome comprises a heterologous polynucleotide comprising a gene encoding an ESR. In some embodiments the heterologous polynucleotide comprises a lentiviral vector, or a piggyBac-like transposon, or a Mariner transposon such as a Sleeping Beauty transposon, or a hAT transposon such as a TcBuster transposon.

In some embodiments a second gene expressed in the immune cell potentiates the effect of the ESR to increase the survival or proliferation of the immune cell. An immune cell whose genome comprises a gene encoding an ESR and a second gene that potentiates the activity of the ESR in increasing the survival or the proliferation of the immune cell (the ESR potentiating gene) is an aspect of the invention. In some embodiments the second gene is operably linked to a heterologous promoter; in some embodiments the second gene encodes an inhibitor of the apoptotic pathway; in some embodiments the inhibitor of the apoptotic pathway is a dominant negative gene in the caspase pathway for example a dominant negative mutant of Caspase 3, Caspase 7, Caspase 8, Caspase 9, Caspase 10 or CASP8 and FADD-like apoptosis regulator (CFLAR); in some embodiments the inhibitor of the apoptotic pathway comprises a dominant negative mutant of a sequence selected from among SEQ ID NO: 240-245; in some embodiments the inhibitor of the apoptotic pathway comprises a sequence selected from among SEQ ID NO: 237, 238 or 261-272.

Preferably the half-life of immune cells expressing an ESR and an ESR-potentiating gene is increased by at least 5%, or at least 10%, or at least 15%, or at least 20%, or at least 25%, or at least 30%, or at least 35%, or at least 40%, or at least 45%, or at least 50%, or at least 55%, or at least 60%, or at least 65%, or at least 70%, or at least 75%, or at least 80%, or at least 85%, or at least 90%, or at least 95%, or at least 100% relative to the half-life of immune cells that are not expressing an immune cell survival-enhancing gene. Preferably the maximum life span of immune cells expressing an ESR and an ESR-potentiating gene is increased by at least 5%, or at least 10%, or at least 15%, or at least 20%, or at least 25%, or at least 30%, or at least 35%, or at least 40%, or at least 45%, or at least 50%, or at least 55%, or at least 60%, or at least 65%, or at least 70%, or at least 75%, or at least 80%, or at least 85%, or at least 90%, or at least 95%, or at least 100% relative to the maximum life span of immune cells that are not expressing an immune cell survival-enhancing gene. Preferably the doubling time of immune cells not expressing an ESR and an ESR-potentiating gene is greater by at least 5%, or at least 10%, or at least 15%, or at least 20%, or at least 25%, or at least 30%, or at least 35%, or at least 40%, or at least 45%, or at least 50%, or at least 55%, or at least 60%, or at least 65%, or at least 70%, or at least 75%, or at least 80%, or at least 85%, or at least 90%, or at least 95%, or at least 100% relative to the doubling time of immune cells that are expressing an ESR and an ESR-potentiating gene. Preferably the proliferation rate of immune cells expressing an ESR and an ESR-potentiating gene is increased by at least 5%, or at least 10%, or at least 15%, or at least 20%, or at least 25%, or at least 30%, or at least 35%, or at least 40%, or at least 45%, or at least 50%, or at least 55%, or at least 60%, or at least 65%, or at least 70%, or at least 75%, or at least 80%, or at least 85%, or at least 90%, or at least 95%, or at least 100% relative to the proliferation rate of immune cells that are not expressing an ESR and an ESR-potentiating gene.

Preferred embodiments comprise a polynucleotide comprising a nucleic acid encoding an inhibitor of apoptosis, wherein the nucleic acid is operably linked to a heterologous promoter. Exemplary heterologous promoters that may be operably linked to the gene encoding an inhibitor of apoptosis include an EF1 promoter, a PGK promoter, a GAPDH promoter, an EEF2 promoter, a ubiquitin promoter, an SV40 promoter or an HSVTK promoter, for example a sequence selected from SEQ ID NOs 94-154. Preferred embodiments comprise a polynucleotide comprising a nucleic acid encoding an inhibitor of apoptosis, wherein the nucleic acid is operably linked to a heterologous polyadenylation signal, for example a polyadenylation signal from a virus that infects mammalian cells, a mammalian EF1 polyadenylation signal, a mammalian growth hormone polyadenylation signal or a mammalian globin polyadenylation signal, for example a sequence selected from SEQ ID NOs 160-217. Preferred embodiments comprise a polynucleotide comprising a gene encoding an inhibitor of apoptosis, wherein the polynucleotide is part of a piggyBac-like transposon which further comprises sequences with SEQ ID NOs: 6 and 7, or sequences with SEQ ID NOs: 14 and 15, or sequences with SEQ ID NOs: 18 and 19, or sequences with SEQ ID NOs: 20 and 21. Preferred embodiments comprise a polynucleotide comprising a gene encoding an inhibitor of apoptosis, wherein the polynucleotide is part of a Mariner transposon such as a Sleeping Beauty transposon which further comprises a sequence that is 90% identical to SEQ ID NO: 24 and a sequence that is 90% identical to SEQ ID NO: 25. Preferred embodiments comprise a polynucleotide comprising a gene encoding an inhibitor of apoptosis, wherein the polynucleotide is part of an hAT transposon such as a TcBuster transposon which further comprises a sequence that is 90% identical to SEQ ID NO: 397 and a sequence that is 90% identical to SEQ ID NO: 398. The piggyBac-like transposon comprising the polynucleotide encoding the inhibitor of apoptosis may introduced into the immune cell together with a polynucleotide encoding a corresponding transposase. Preferred embodiments comprise a polynucleotide comprising a gene encoding an inhibitor of apoptosis, wherein the polynucleotide is part of a lentiviral vector. The lentiviral vector comprising the polynucleotide encoding the inhibitor of apoptosis may be packaged and used to infect the immune cell. The immune cell is preferably a T-cell or a B-cell.

One aspect of the present invention is an immune cell whose genome comprises a heterologous polynucleotide comprising a gene encoding an inhibitor of apoptosis. In some embodiments the heterologous polynucleotide comprises a lentiviral vector, or a piggyBac-like transposon, or a Mariner transposon such as a Sleeping Beauty transposon, or an hAT transposon such as a TcBuste transposon.

Optionally the polynucleotide comprising a gene encoding the ESR further comprises a second gene encoding an inhibitor of apoptosis operably linked to a heterologous promoter.

### FAS/4-1BB

As an exemplary ESR in which the extracellular domain of an inhibitory receptor is fused to the intracellular domain of a co-stimulatory receptor, we designed an ESR comprising the extracellular domain of TNFRSF6 (Fas) (SEQ ID NO: 323), and further comprising the transmembrane domain of TNFRSF6 (Fas) (SEQ ID NO: 387) and further comprising the intracellular domain of TNFRSF9 (4-1BB) (SEQ ID NO: 344). This ESR (Fas/4-1BB) comprising sequence SEQ ID NO: 274 is an immune cell survival-enhancing gene and an immune cell proliferation-enhancing gene as described in Section 6.2. The activity of Fas/4-1BB is potentiated by an inhibitor of apoptosis: a dominant negative version of Casp7: Casp7-DN (SEQ ID NO: 262). The effectiveness of Fas/4-1BB plus Casp7-DN in enhancing immune cell survival and immune cell proliferation is shown in Sections 6.2.1.2 and 6.2.2.2.

A polynucleotide comprising a gene encoding Fas/4-1BB (SEQ ID NO: 274) is an aspect of the invention. An immune cell whose genome comprises a gene encoding Fas/4-1BB is an aspect of the invention. A polynucleotide comprising a gene encoding Fas/4-1BB and further comprising a gene encoding an inhibitor of apoptosis is an aspect of the invention; in some embodiments the inhibitor of apoptosis is a dominant negative mutant of Casp 7, for example SEQ ID NO: 262. Preferably the polynucleotide is a transposon. An immune cell whose genome comprises a gene encoding Fas/4-1BB and a dominant negative inhibitor of apoptosis is an aspect of the invention. Such an immune cell is particularly advantageous for ex-vivo growth in cell culture.

Anti-CD28/OX40 is an ESR with Proliferation-Enhancing Activity

As an exemplary ESR in which the extracellular domain comprises the binding domain from an antibody which is fused to the intracellular domain of a co-stimulatory receptor, we designed an ESR whose extracellular domain comprised the binding domain of CD28 agonist antibody TGN1412 (SEQ ID NO: 340) fused to the transmembrane domain for TNFRSF4 (OX40) (SEQ ID NO: 373) and the intracellular domain for TNFRSF4 (OX40) (SEQ ID NO: 341). The sequence of the anti-CD28/OX40 ESR is given as (SEQ ID NO: 307). The effectiveness of this ESR in promoting T-cell proliferation is described in Section 6.2.2.1.

A polynucleotide comprising a gene encoding an anti-CD28/OX40 ESR (for example SEQ ID NO: ESR34) is an aspect of the invention. An immune cell whose genome comprises a gene encoding an anti-CD28/OX40 ESR is an aspect of the invention. Such an immune cell is particularly advantageous for ex-vivo growth in cell culture.

The present invention also features kits comprising a transposase as a protein or encoded by a nucleic acid, and/or a transposon; or a gene transfer system as described herein comprising a transposase as a protein or encoded by a nucleic acid as described herein, in combination with a transposon; optionally together with a pharmaceutically acceptable carrier, adjuvant or vehicle, and optionally with instructions for use. Any of the components of the inventive kit may be administered and/or transfected into cells in a subsequent order or in parallel, e.g. a transposase protein or its encoding nucleic acid may be administered and/or transfected into a cell as defined above prior to, simultaneously with or subsequent to administration and/or transfection of a transposon. Alternatively, a transposon may be transfected into a cell as defined above prior to, simultaneously with or subsequent to transfection of a transposase protein or its encoding nucleic acid. If transfected in parallel, preferably both components are provided in a separated formulation and/or mixed with each other directly prior to administration to avoid transposition prior to transfection. Additionally, administration and/or transfection of at least one component of the kit may occur in a time staggered mode, e.g. by administering multiple doses of this component.

### EXAMPLES

The following examples illustrate the methods, compositions and kits disclosed herein and should not be construed as limiting in any way. Various equivalents will be apparent from the following examples; such equivalents are also contemplated to be part of the invention disclosed herein.

### Gene Transfer System Elements for Expression in Immune Cells

### Transposon Elements in a Human T-Cell Line

Jurkat cells are an immortalized line of human T-cells, they are useful for testing gene transfer systems for their effectiveness in human immune cells, particularly T-cells. We tested the ability of Xenopus and Bombyx piggyBac-like transposases to transpose their corresponding transposons into the genome of the Jurkat human T-cell line.

A polynucleotide (CD19-GFP-LPN1, with nucleotide sequence given by SEQ ID NO: 223) comprising a Xenopus transposon was constructed in which a nucleic acid encoding CD19 (with amino acid sequence given by SEQ ID NO: 228) was operably linked to an EF1 promoter with sequence given by SEQ ID NO: 94 and a bovine growth hormone polyadenylation signal sequence with SEQ ID NO: 174. The CD19 gene was flanked on one side by an HS4 insulator (SEQ ID NO: 92), and on the other by a D4Z4 insulator (SEQ ID NO: 88). The gene transfer polynucleotide further comprised, on the distal side of one insulator, a target sequence 5′-TTAA-3′, immediately followed by a piggyBac-like transposon inverted terminal repeat sequence SEQ ID NO: ITR 8 (which is an embodiment of SEQ ID NO: 6), immediately followed by additional transposon end sequences with SEQ ID NO: 1. The gene transfer polynucleotide further comprised, on the distal side of the other insulator, additional transposon end sequences with SEQ ID NO: 4, immediately followed by a piggyBac-like transposon inverted terminal repeat sequence SEQ ID NO: 9 (which is an embodiment of SEQ ID NO: 7) immediately followed by a target sequence 5′-TTAA-3′. The transposon further comprised a polynucleotide encoding GFP operably linked to a CMV promoter and a bovine growth hormone polyadenylation signal sequence. The CD19 and GFP genes were placed such that transposition of the piggyBac-like Xenopus transposon by its corresponding transposase transposes the CD19 gene, but leaves the GFP gene behind in the plasmid.

A polynucleotide (CD19-RFP-LPN2, with nucleotide sequence given by SEQ ID NO: 224) comprising a Bombyx transposon was constructed in which a gene encoding CD19 (SEQ ID NO: 228) was operably linked to an EF1 promoter with sequence given by SEQ ID NO: 94 and a bovine growth hormone polyadenylation signal sequence with SEQ ID NO: 174. The CD19 gene was flanked on one side by an HS4 insulator (SEQ ID NO: 92), and on the other by a D4Z4 insulator (SEQ ID NO: 88). The gene transfer polynucleotide further comprised, on the distal side of one insulator, a target sequence 5′-TTAA-3′, immediately followed by a piggyBac-like transposon inverted terminal repeat sequence SEQ ID NO: 14, immediately followed by additional transposon end sequences with SEQ ID NO: 12. The gene transfer polynucleotide further comprised, on the distal side of the other insulator, additional transposon end sequences with SEQ ID NO: 13, immediately followed by a piggyBac-like transposon inverted terminal repeat sequence SEQ ID NO: 15 immediately followed by a target sequence 5′-TTAA-3′. The transposon further comprised a polynucleotide encoding RFP operably linked to a CMV promoter and a bovine growth hormone polyadenylation signal sequence. The CD19 and RFP genes were placed such that transposition of the piggyBac-like Bombyx transposon by its corresponding transposase transposes the CD19 gene, but leaves the RFP gene behind in the plasmid.

One sample of Jurkat cells (200,000 cells per transfection) was transfected with 1 ng of CD19-GFP-LPN1 plasmid DNA and 100 ng of transposase mRNA encoding Xenopus transposase with sequence given by SEQ ID NO: 37, using a Neon electroporator according to the manufacturer's instructions. A second sample of Jurkat cells (200,000 cells per transfection) were transfected with 1 μg of CD19-RFP-LPN2 plasmid DNA and 100 ng of Bombyx transposase mRNA encoding transposase with sequence given by SEQ ID NO: 68, using a Neon electroporator according to the manufacturer's instructions. After various times, cells were labeled with an anti-CD19 antibody and the percentage of cells expressing CD19 was determined by flow cytometry. The results are shown in Table 1. Initially about 85% of the transfected cells showed CD19 expression (Table 1 row 1). This corresponds to a combination of expression from plasmid that has been taken up into the cells, and from transposons that have been stably integrated into the T-cell line genome. Over the next 10 days, the percentage of cells expressing CD19 fell to 18% for cells transfected with the Xenopus piggyBac-like transposon and 27% for cells transfected with the Bombyx piggyBac-like transposon (Table 1 row 3). This corresponds to loss of expression from cells in which CD19 genes have not been integrated into the genome. From about 15 days post-transfection until at least 55 days post-transfection, the percentage of cells expressing CD19 remained approximately constant (Table 1 rows 3-6). During all this time cells were growing and dividing. The plasmid has no way to replicate in a human cell, so the only way for a cell to keep expressing CD19 is if the CD19 gene is integrated into the genome which then replicates the CD19 gene at each cell division along with the rest of the genome. The rate of integration through random fragmentation is very low: between 0.01 and 1% of cells might be expected to integrate transfected DNA. The percentage of cells that integrated the CD19 gene was much higher than the frequency that would be expected to result from random integration, but consistent with the frequency that might be expected from transposition. Cells were also analyzed for the expression of GFP and RFP. By day 55 there was no detectable GFP or RFP expression. As described above, the GFP and RFP genes were placed on a part of the gene transfer plasmid that was not transposable by the transposase. GFP and RFP expression would therefore be expected if the gene transfer plasmids had integrated into the cell genome by random fragmentation and integration. However, if the CD19 gene integrated as a result of transposition, the GFP or RFP gene would be left behind in the plasmid and would be gradually degraded over time. The high genomic integration frequency and the lack of expression of non-transposable genes lead us to conclude that gene transfer systems based on the Xenopus and Bombyx piggyBac-like transposon systems are both capable of integrating polynucleotides into human T-cells.

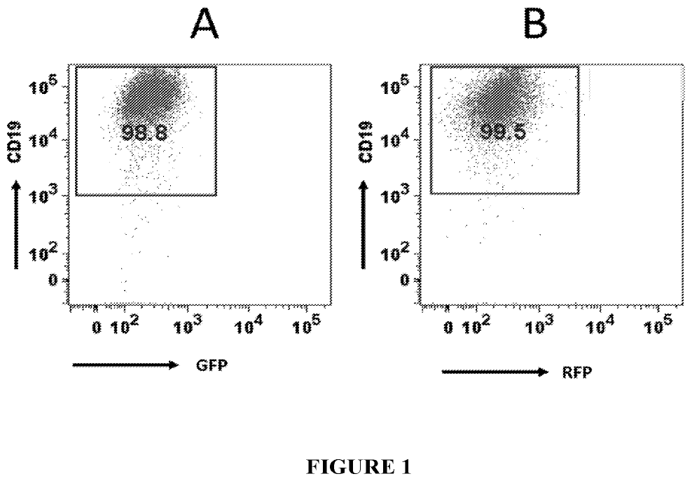

At 70 days post-transfection, we used fluorescence activated cell sorting to sort cells expressing CD19 from those that were not expressing CD19. These cells were then maintained in liquid culture for over 240 days and analyzed at various times to assess the stability of integration. FIG. 1 shows the expression of CD19 on the y-axis, and the expression of the fluorescent protein on the x-axis, 155 days post-transfection and 85 days post-FACS sorting. Essentially all cells were still expressing CD19, and no cells were expressing a fluorescent protein. Identical results were obtained 240 days post-transfection. We conclude that Xenopus and Bombyx piggyBac-like transposons are stably maintained, even in the absence of selective pressure, for at least 240 days. We also note that the maintenance of expression indicates that the gene encoded on the transposon has not been silenced during more than 200 cell generations. Gene transfer systems based on the Xenopus and Bombyx piggyBac-like transposon systems are thus both useful for delivering genes for expression into human T-cells.

### Promoter Elements in T-Cells

Promoter test in Jurkat Cells

Jurkat cells are an immortalized line of human T-cells, they are useful for testing gene transfer systems for their effectiveness in human immune cells, particularly T-cells.

Promoter elements, optionally combined with intron elements, were cloned into a transposon for expression of human CD19, by introducing them between a first polynucleotide with sequence given by SEQ ID NO: 218 and a second polynucleotide with sequence given by SEQ ID NO: 219 to generate a circular plasmid comprising an insulator sequence with sequence given by SEQ ID NO: 90, an insulator sequence with sequence given by SEQ ID NO: 93, and flanked by a pair of transposon ends, one comprising sequence SEQ ID NO: 8 which is an embodiment of SEQ ID NO: 6 and one comprising sequence SEQ ID NO: 9 which is an embodiment of SEQ ID NO: 7. Jurkat cells (200,000 cells per transfection) were transfected with 1 μg of plasmid DNA and 100 ng of transposase mRNA encoding Xenopus transposase with amino acid sequence SEQ ID NO: 37, using a Neon electroporator according to the manufacturer's instructions.

Samples were taken for FACS analysis at various times after transfection, and the fraction of live cells expressing CD19 on their surfaces was counted and is shown in Table 2. Table 2 shows that cells into which CD19 was operably linked to EF1 (e.g. SEQ ID NOs: 94 and 132) and EEF2 (e.g. SEQ ID NO: 108) showed a high initial percentage of CD19-expressing cells (column D), but this was not sustained (e.g. columns F and G). For example, the percentage of cells expressing rat EF1-driven CD19 fell from 87% to 25% between day 2 and day 23, similarly the percentage of cells expressing human EF1-driven CD19 fell from 76% to 37% between day 2 and day 23. In contrast, when CD19 was operably linked to GAPDH, ubiquitin and PGK promoters, the cells showed much more consistently sustained levels of expression, with about 50% of cells expressing CD19 at each sample time between day 2 and day 23 (columns D-G). Column H shows the percentage decline in CD19-expressing cells between day 2 and day 23.

CD19 is a molecule expressed on the cell surface. Substantial over-expression of transmembrane proteins can be toxic. We therefore reasoned that the promoters that showed the most dramatic losses of CD19-expressing cells might be those that were driving the strongest expression. To assess promoter strength, we operably linked each of the promoters to a gene encoding GFP and transfected the genes in triplicate into HEK cells. After 2 days the fluorescence was measured in a fluorimeter. The average fluorescence values are shown in Table 2, column I. The strongest promoters were EF1 and EEF2 (column I, rows 1, 2 and 6), and these were the same promoters that showed the most substantial declines in the percentage of CD19-expressing cells (Table 2 column H). In contrast the PGK, GAPDH and ubiquitin promoters were only 8.6%, 28% and 22% as active as the strongest EF1 promoter, but the percentage of cells expressing CD19 operably linked to these promoters was sustained. Moderately active promoters thus appear advantageous over highly active promoters for the expression of genes encoding transmembrane proteins in T-cells, as they produce high enough levels of transmembrane protein to achieve function without causing toxicity. Transmembrane proteins include T-cell receptors, chimeric antigen receptors and enhanced signaling receptors. Moderately active promoters include phosphoglycerate kinase promoters, glyceraldehyde-3-phosphate dehydrogenase promoters and ubiquitin promoters. They may also include highly active promoters that have been attenuated, for example by removal of an intron or partial deletion of the promoter, such as an attenuated EF1 promoter or an attenuated EEF2 promoter.

This is an unexpected result: most current work in expressing chimeric antigen receptors is performed with strongly active promoters such as CMV or EF1 promoters. In contrast, here we have found that such highly active promoters are disadvantageous when operably linked to a transmembrane protein. An advantageous gene transfer system for expression of genes encoding transmembrane proteins in a T-cell comprises a polynucleotide comprising a gene encoding the transmembrane protein operably linked to a promoter selected from a phosphoglycerate kinase promoter, a glyceraldehyde-3-phosphate dehydrogenase promoter a ubiquitin promoter, an attenuated EF1 promoter or an attenuated EEF2 promoter. Exemplary phosphoglycerate kinase promoter sequences are given as SEQ ID NO: 115-118. Exemplary glyceraldehyde-3-phosphate dehydrogenase promoter sequences are given as SEQ ID NO: 97-107. Exemplary ubiquitin promoter sequences are given as SEQ ID NO: 95 and 125-127. The polynucleotide may further comprise an insulator sequence selected from SEQ ID NO: 87-93. Preferably the gene transfer polynucleotide comprises transposon ends such that it is recognized and transposed by a corresponding transposase, that such transposition may insert the promoter and its operably linked gene into the genome of an immune cell such as a T-cell. The polynucleotide may be part of a piggyBac-like transposon which further comprises sequences with SEQ ID NOs: 6 and 7, or sequences with SEQ ID NOs: 14 and 15, or sequences with SEQ ID NOs: 18 and 19, or sequences with SEQ ID NOs: 20 and 21. The polynucleotide may be part of a Mariner transposon such as a Sleeping Beauty transposon which further comprises a sequence that is 90% identical to SEQ ID NO: 24 and a sequence that is 90% identical to SEQ ID NO: 25. The polynucleotide may be part of an hAT transposon such as a TcBuster transposon which further comprises a sequence that is 90% identical to SEQ ID NO: 397 and a sequence that is 90% identical to SEQ ID NO: 398. The experiment was repeated, and after 8 days, cells were labeled with an anti-CD19 antibody and mean fluorescent intensity of cells expressing CD19 at day 8 was measured by flow cytometry. Mean fluorescent intensity values are shown in Table 3 column D. For comparison, human B-cells which naturally express CD19 at around 22,000 molecules per cell were labeled and mean fluorescent intensity was measured. The mean fluorescent intensity of B cells was used to calculate the number of CD19 molecules expressed on the surfaces of the Jurkat cells, as shown in Table 3 column E. For all promoters tested, the number of CD19 molecules on the surface of each cell was between 2 and 5 times the number naturally found on B-cells. The mean fluorescent intensity of cells transfected with CD19 operably linked to an EF1 promoter was within 20% of the value of cells transfected with CD19 operably linked to a PGK promoter (compare Table 3 rows 5 and 6), even though the PGK promoter is known to be much less active than the EF1 promoter, for example as shown in Table 2 column I rows 5 and 6. We interpret this to be because cells in which expression of CD19 exceeds ˜5 times the number normally found on the surface of B-cells experience toxicity. EF1 is a stronger promoter, so a higher fraction of cells transfected with CD19 operably linked to an EF1 promoter exceed this toxicity limit and die. This results in the loss of CD19-expressing cells observed between days 2 and 8 in Table 2 row 6. Moderately active promoters are thus capable of producing high levels of expression of transmembrane proteins, but the level is less likely to be so high as to be toxic. This is advantageous in transfection of T-cells with transmembrane proteins such as chimeric antigen receptors.

Table 3 shows that promoters with sequences given by SEQ ID NOs 94, 95, 98, 108, 115 and 132 were all effective in driving high levels of CD19 expression in Jurkat immortalized T-cells. An advantageous gene transfer system for expression of genes in a T-cell comprises a polynucleotide comprising a promoter with a sequence selected from SEQ ID NO: 94, 95, 98, 108, 115 and 132. An advantageous gene transfer system for expression of genes in a T-cell comprises a polynucleotide comprising an insulator sequence selected from SEQ ID NO: 87-91, and an insulator sequence selected from SEQ ID NO: 92 and 93. An advantageous gene transfer system for expression of genes in a T-cell comprises a polynucleotide comprising a transposon end comprising sequence SEQ ID NO: 6 and a transposon end comprising sequence SEQ ID NO: 7.

### Promoter Test in Primary T-Cells

Promoter elements were cloned into a transposon for expression of human CD19, by introducing them between a first polynucleotide with sequence given by SEQ ID NO: 220 and a second polynucleotide with sequence given by SEQ ID NO: 221 to generate a circular plasmid comprising an insulator sequence with sequence SEQ ID NO: 88, an insulator sequence with SEQ ID NO: 92, and flanked by a pair of transposon ends, one comprising target site 5′-TTAA-3′ immediately followed by sequence SEQ ID NO: 8 immediately followed by sequence SEQ ID NO: 1 and the other comprising sequence SEQ ID NO: 4 immediately followed by sequence SEQ ID NO: 9, immediately followed by target site 5′-TTAA-3′. Primary T-cells (200,000 cells per transfection) were transfected with 1 μg of plasmid DNA and 100 ng of transposase mRNA encoding Xenopus transposase with polypeptide sequence SEQ ID NO: 37, using a Neon electroporator according to the manufacturer's instructions. After 11 days, cells were labeled with an anti-CD19 antibody and mean fluorescent intensity was measured by flow cytometry.

Table 4 shows that promoters with sequences given by SEQ ID NOs: 97, 98 and 108-114 were all effective in driving high levels of CD19 expression in primary T-cells. It further shows that different levels of expression can be achieved by using different promoters. An advantageous gene transfer system for expression of genes in a T-cell comprises a polynucleotide comprising a promoter with a sequence selected from SEQ ID NO: 97, 98 and 108-114. An advantageous gene transfer system for expression of genes in a T-cell comprises a polynucleotide comprising a transposon end comprising sequence SEQ ID NO: 8 immediately followed by sequence SEQ ID NO: 1 and a transposon end comprising SEQ ID NO: 4 immediately followed by sequence SEQ ID NO: 9.

### Elements for Enhancing Survival and Efficacy of Immune Cells

An aspect of the present invention is the disclosure of sequences that can be used to enhance the survival, proliferation or expansion of immune cells.

Cell survival can be measured as the length of time that it takes for only half of the cells in a population to remain alive (the half-life), or the time it takes all the cells in a population to die (the maximum life span). Immune cells expressing an immune cell survival-enhancing gene will remain alive for longer than immune cells that are not expressing an immune cell survival-enhancing gene. One way of measuring this effect is to integrate a heterologous polynucleotide into the genome of the immune cell, wherein the heterologous polynucleotide comprises the immune cell survival-enhancing gene operably linked to regulatory sequences that cause it to be expressed within the immune cell, in other words is effective for expression in an immune cell. The heterologous polynucleotide further comprises a gene encoding a selectable marker, for example one that can be readily identified such as a fluorescent protein or a cell surface protein. Cells whose genomes comprise the heterologous polynucleotide express the immune cell survival-enhancing gene, and they can be identified by the presence of the selectable marker. Enhancement of survival can be measured as an increase in the half-life of immune cells expressing the immune cell survival-enhancing gene relative to immune cells that are not expressing the immune cell survival-enhancing gene. The half-life of immune cells expressing an immune cell survival-enhancing gene is increased by at least 5%, or at least 10%, or at least 15%, or at least 20%, or at least 25%, or at least 30%, or at least 35%, or at least 40%, or at least 45%, or at least 50%, or at least 55%, or at least 60%, or at least 65%, or at least 70%, or at least 75%, or at least 80%, or at least 85%, or at least 90%, or at least 95%, or at least 100% relative to the half-life of immune cells that are not expressing an immune cell survival-enhancing gene. The increase can be measured by comparing survival in equal size populations of a particular immune cell with and without a survival-enhancing gene. The maximum life span of immune cells expressing an immune cell survival-enhancing gene is increased by at least 5%, or at least 10%, or at least 15%, or at least 20%, or at least 25%, or at least 30%, or at least 35%, or at least 40%, or at least 45%, or at least 50%, or at least 55%, or at least 60%, or at least 65%, or at least 70%, or at least 75%, or at least 80%, or at least 85%, or at least 90%, or at least 95%, or at least 100% relative to the maximum life span of immune cells that are not expressing an immune cell survival-enhancing gene. Percentage changes in maximum lifespan can be measured by comparing equal sized populations of a particular immune cell with and without a survival-enhancing gene

Cell proliferation can be measured as the length of time that it takes the number of cells in a population to double (the doubling time), or as the fraction by which a cell population increases in a unit length of time (the proliferation rate). Immune cells expressing an immune cell proliferation-enhancing gene may divide for longer, or they may divide more rapidly than immune cells that are not expressing an immune cell proliferation-enhancing gene. One way of measuring this effect is to integrate a heterologous polynucleotide into the genome of the immune cell, wherein the heterologous polynucleotide comprises an immune cell proliferation-enhancing gene operably linked to regulatory sequences that cause it to be expressed within the immune cell. The heterologous polynucleotide further comprises a gene encoding a selectable marker, for example one that can be readily identified such as a fluorescent protein or a cell surface protein. Cells whose genomes comprise the heterologous polynucleotide express the immune cell proliferation-enhancing gene, and they can be identified by the presence of the selectable marker. Enhancement of proliferation can be measured as a decrease in the doubling time of immune cells expressing the immune cell proliferation-enhancing gene relative to immune cells that are not expressing the immune cell proliferation-enhancing gene. The doubling time of immune cells not expressing an immune cell proliferation-enhancing gene is greater by at least 5%, or at least 10%, or at least 15%, or at least 20%, or at least 25%, or at least 30%, or at least 35%, or at least 40%, or at least 45%, or at least 50%, or at least 55%, or at least 60%, or at least 65%, or at least 70%, or at least 75%, or at least 80%, or at least 85%, or at least 90%, or at least 95%, or at least 100% relative to the doubling time of immune cells that are expressing an immune cell proliferation-enhancing gene. The proliferation rate of immune cells expressing an immune cell proliferation-enhancing gene is increased by at least 5%, or at least 10%, or at least 15%, or at least 20%, or at least 25%, or at least 30%, or at least 35%, or at least 40%, or at least 45%, or at least 50%, or at least 55%, or at least 60%, or at least 65%, or at least 70%, or at least 75%, or at least 80%, or at least 85%, or at least 90%, or at least 95%, or at least 100% relative to the proliferation rate of immune cells that are not expressing an immune cell proliferation-enhancing gene. The proliferation rate or the doubling time may be measured at various times after the immune cell has begun expressing the immune cell proliferation-enhancing gene. The proliferation rate of immune cells expressing an immune cell proliferation-enhancing gene may be increased relative to the proliferation rate of the same immune cells that are not expressing an immune cell proliferation-enhancing gene 5 days after, or 10 days after, or 15 days after, or 20 days after, or 25 days after, or 30 days after, or 35 days after, or 40 days after, or 45 days after, or 50 days after, or 55 days after, or 60 days after the heterologous polynucleotide is integrated into the genome of the immune cells, or after the immune cells begin expressing the immune cell proliferation-enhancing gene.

Another aspect of the present invention is the disclosure of sequences that can be used to increase the length of time that immune cells can remain effective under conditions that reduce the efficacy of normal immune cells. Normal T-cells undergo apoptosis when repeatedly exposed to an antigen (“restimulation-induced cell death”), and those that do not die become unable to kill cells expressing the antigen (Voss et. al. (2017) Cancer Lett. 408: 190-196. “Metabolic reprogramming and apoptosis sensitivity: defining the contours of a T cell response”). Although this helps to reduce auto-immunity, it has been a contributing factor in preventing T cells from effectively combatting solid tumors. Under these circumstances it is thus desirable to retain immune cell function and prevent restimulation-induced cell death during repeated antigen exposure. Restimulation-induced cell death may be measured by counting the number of T-cells surviving after 2, 3, 4 or more exposures to an antigen, for example an antigen on a tumor cell. The ability of a heterologously expressed sequence to prevent restimulation-induced cell death may be measured by comparing the survival of T-cells expressing the sequence with the survival of T-cells that are not expressing the sequence, when both populations have the same extent and frequency of antigen exposure. Enhancement of survival can be measured as an increase in the number of remaining immune cells expressing the immune cell survival-enhancing gene relative to immune cells that are not expressing the immune cell survival-enhancing gene upon repeated exposure to an antigen. The number of surviving immune cells expressing an immune cell survival-enhancing gene is increased by at least 10%, or at least 20%, or at least 30%, or at least 40%, or at least 50%, or at least 60%, or at least 70%, or at least 80%, or at least 90%, or at least 100%, or at least 150%, or at least 200%, or at least 250%, or at least 300%, or at least 350%, or at least 400%, or at least 450%, or at least 500% relative to the number of surviving immune cells that are not expressing an immune cell survival-enhancing gene upon repeated exposure to an antigen for example expressed in a tumor cell.

Resistance to restimulation-induced cell death and sustained immune cell efficacy may be measured by counting the ability of T-cells to kill a cell such as a tumor cell after 2, 3, 4 or more exposures to the tumor cell. The ability of a heterologously expressed sequence to sustain immune cell function may be measured by comparing the cell killing activity of T-cells expressing the sequence with the cell killing activity of T-cells that are not expressing the sequence, when both populations have the same extent and frequency of antigen exposure. The cell killing activity of T-cells expressing a T-cell efficacy-enhancing gene is increased by at least 10%, or at least 20%, or at least 30%, or at least 40%, or at least 50%, or at least 60%, or at least 70%, or at least 80%, or at least 90%, or at least 100%, or at least 150%, or at least 200%, or at least 250%, or at least 300%, or at least 350%, or at least 400%, or at least 450%, or at least 500% relative to the number of surviving immune cells that are not expressing the T-cell efficacy-enhancing gene upon repeated exposure to a tumor cell.

### T-Cell Transformation Elements

### Expression of Mutated STAT3 in Primary T-Cells

A gene encoding a mutated version of STAT3: STAT3-Y640F was operably linked to a PGK promoter with sequence given by SEQ ID NO: 115 and a rabbit globin polyadenylation signal with sequence SEQ ID NO: 182 and cloned into a gene transfer polynucleotide. The gene transfer polynucleotide further comprised a GFP reporter (with sequence SEQ ID NO: 222) comprising a gene encoding DasherGFP operably linked to a glyceraldehyde-3-phosphate dehydrogenase (GAPDH) promoter and a bovine growth hormone (BGH) polyadenylation signal sequence. The two open reading frames were configured to be divergently transcribed (the two promoters were adjacent to each other and transcribed in opposite directions). The two open reading frames were flanked on one side by an HS4 insulator (with sequence SEQ ID NO: 92), and on the other by a D4Z4 insulator (with sequence SEQ ID NO: 88). The gene transfer polynucleotide further comprised, on the distal side of one insulator, a target sequence 5′-TTAA-3′, immediately followed by a piggyBac-like transposon inverted terminal repeat sequence SEQ ID NO: 10 (which is an embodiment of SEQ ID NO: 6), immediately followed by additional transposon end sequences SEQ ID NO: 3 (which is >95% identical to SEQ ID NO: 1). The gene transfer polynucleotide further comprised, on the distal side of the other insulator, additional transposon end sequences SEQ ID NO: 5 (which is >95% identical to SEQ ID NO: 4), immediately followed by a piggyBac-like transposon inverted terminal repeat sequence SEQ ID NO: 11 (which is an embodiment of SEQ ID NO: 7) immediately followed by a target sequence 5′-TTAA-3′.

T-cells were prepared from normal donor peripheral blood mononuclear cells (PBMCs) using the EasySep Human CD8 positive selection kit from Stemcell Technologies according to the manufacturer's instructions. T-cells were stimulated for 2-3 days by incubation with irradiated feeder cells to provide secreted CD3, CD28, IL-2, IL-7 and IL-15. Approximately 100,000 T-cells were transfected with 1 μg transposon DNA and 100 ng mRNA encoding transposase with polypeptide sequence SEQ ID NO: 37 using a Neon electroporator according to the manufacturer's instructions. Transfected T-cells were mixed with feeder cells and incubated at 37° C. Samples were taken at various times post-transfection, incubated with a fluorescently-labelled anti-CD8 antibody, and analyzed on a fluorescence-activated cell sorter (FACS) for CD8 and Dasher GFP.

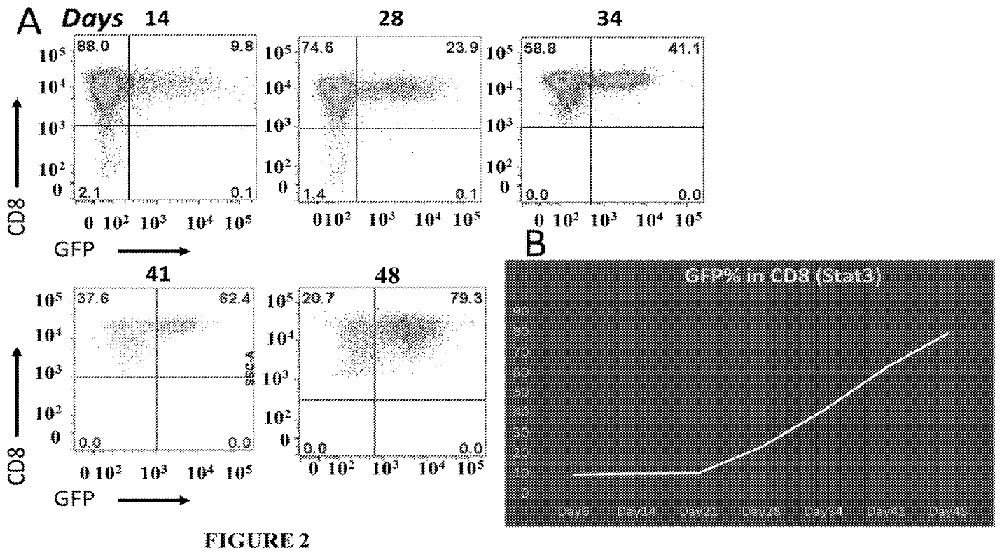

FIG. 2 shows the distribution of cell staining over time. CD8 staining is used as a marker for CD8+ T-cells, and is shown on the y-axis of each of the FACS plots shown in Panel A. GFP fluorescence is shown on the x-axis of each FACS plot; GFP fluorescence indicates that the cell is expressing GFP, and is also used here as a marker to indicate the presence of the gene transfer polynucleotide within the cell. On day 14, approximately 97.8% of the cells showed strong CD8-staining (i.e. they are in the upper half of the FACS plot), and approximately 9.8% of the analyzed cells were both CD8+ and showed GFP fluorescence. The fraction of cells expressing CD8 and exhibiting GFP fluorescence increased over time: 23.9% at day 28, 41.1% at day 34, 62.4% at day 41 and 79.3% at day 48. The increase in the fraction of the T-cell population expressing GFP either indicates that the T-cells whose genomes comprise the gene transfer polynucleotide possess a survival advantage compared with the T-cells whose genomes do not comprise the gene transfer polynucleotide, or it indicates that the T-cells whose genomes comprise the gene transfer polynucleotide possess a proliferation advantage compared with the T-cells whose genomes do not comprise the gene transfer polynucleotide. Such survival or proliferation advantage originates not in the expression of GFP (we see many examples where GFP expression does not correlate with a survival or proliferation advantage), but in the expression of STAT3-Y640F. We conclude that expression of STAT3-Y640F in T-cells provides them with a survival or proliferation advantage, and that a gene encoding an activating mutant of STAT3 is an immune cell survival-enhancing gene and an immune cell proliferation-enhancing gene as described in Section 5.3.1.1.

### Expression of T-Cell Transformation Elements and ESRs in Primary T-Cells

Genes encoding a set of T-cell transformation elements and enhanced signaling receptors were cloned individually into separate gene transfer polynucleotides. In each case the gene was operably linked to a PGK promoter with sequence given by SEQ ID NO: 115 and a rabbit globin polyadenylation signal with sequence SEQ ID NO: 182 and cloned into a gene transfer polynucleotide. The gene transfer polynucleotide further comprised a GFP reporter (with sequence SEQ ID NO: 222) comprising a gene encoding DasherGFP operably linked to a glyceraldehyde-3-phosphate dehydrogenase (GAPDH) promoter and a bovine growth hormone (BGH) polyadenylation signal sequence. The two open reading frames were configured to be divergently transcribed (the two promoters were adjacent to each other and transcribed in opposite directions). The two open reading frames were flanked on one side by an HS4 insulator (with sequence SEQ ID NO: 92), and on the other by a D4Z4 insulator (with sequence SEQ ID NO: 88). The gene transfer polynucleotide further comprised, on the distal side of one insulator, a target sequence 5′-TTAA-3′, immediately followed by a piggyBac-like transposon inverted terminal repeat sequence SEQ ID NO: 10 (which is an embodiment of SEQ ID NO: 6), immediately followed by additional transposon end sequences SEQ ID NO: 3 (which is >95% identical to SEQ ID NO: 1). The gene transfer polynucleotide further comprised, on the distal side of the other insulator, additional transposon end sequences SEQ ID NO: 5 (which is >95% identical to SEQ ID NO: 4), immediately followed by a piggyBac-like transposon inverted terminal repeat sequence SEQ ID NO: 11 (which is an embodiment of SEQ ID NO: 7) immediately followed by a target sequence 5′-TTAA-3′.

T-cells were prepared from normal donor peripheral blood mononuclear cells (PBMCs) using the EasySep Human CD8 positive selection kit from Stemcell Technologies according the manufacturer's instructions. T-cells were stimulated for 2-3 days by incubation with irradiated feeder cells to provide secreted CD3, CD28, IL-2, IL-7 and IL-15. Approximately 100,000 T-cells were transfected with 1 μg transposon DNA and 100 ng mRNA encoding transposase with polypeptide sequence SEQ ID NO: 37 using a Neon electroporator according to the manufacturer's protocol. Transfected T-cells were mixed with feeder cells and incubated at 37° C. Samples were taken at 24 days post-transfection, incubated with a fluorescently-labelled anti-CD8 antibody, and analyzed on a fluorescence-activated cell sorter (FACS) for CD8 and Dasher GFP. The data is shown in Table 5.

As described in Section 6.2.1.1, the enrichment of CD8+ cells expressing GFP is an indicator that the gene transfer polynucleotide comprises a gene that confers a survival or a proliferation advantage to a T-cell, as also described in Section 5.3.1.1. In this set of gene transfer polynucleotides, CD19 was included as a cell-surface marker that is expected to have no effect on T-cell survival, we therefore used the percentage of cells expressing GFP in cells transfected with CD19 (3%, see Table 5 row 10) as a level against which to benchmark the putative survival-enhancing genes. T-cells transfected with a gene encoding CD28 with two activating mutations D124E and T195P had more than 10-times as many cells expressing GFP (Table 5 row 1) than did the cells transfected with CD19. Genes encoding an antibody fragment recognizing the epidermal growth factor receptor (EGFR) fused to CD3e or CD3d appeared to confer a 4-fold increase in the percentage of GFP expressing cells compared with CD19 (Table 5 rows 2 and 3 respectively), although we note that a second measurement of the CD3d fusion showed a much lower percentage of GFP expression (Table 5 row 15). The natural Survivin gene appeared to confer a 3-fold increase in GFP expression compared with CD19 (Table 5 rows 4 and 5). Two ESRs are also shown. The first, comprising an anti-CD28 antibody (with sequence SEQ ID NO: 340) fused to the CD28 transmembrane domain (with sequence SEQ ID NO: 395) and the CD28 intracellular domain comprising the T195P activating mutation (with sequence SEQ ID NO: 352), led to about a 2-fold increase in the number of GFP-expressing cells (Table 5 rows 6 and 7). The second ESR, comprising a TNFRSF1A extracellular domain (with sequence SEQ ID NO: 330) and transmembrane domain (with sequence SEQ ID NO: 394) and the 4-1BB intracellular domain (with sequence SEQ ID NO: 344) resulted in a little less than 2-fold increase in the number of GFP-expressing cells (Table 5 rows 8 and 9). Two co-transfections were particularly active in increasing the percentage of cells expressing GFP. One co-transfection, shown in Table 5 row 16, comprised a first gene encoding an ESR (also described in Section 5.3.2.1) comprising the extracellular domain and transmembrane domain of the Fas receptor (TNFRSF6) (with sequences SEQ ID NOs: 323 and 387 respectively) and the intracellular domain of 4-1BB (TNFRSF9) (with sequence SEQ ID NO: 344), and a second gene encoding a dominant negative mutant of caspase 7 (with sequence SEQ ID NO: 262). The second co-transfection, shown in Table 5 row 17, comprised a first gene encoding STA3-Y640F (with sequence SEQ ID NO: 246) and a second gene encoding PIK3CA-L1001P (with sequence SEQ ID NO: 257). These co-transfections resulted in 51% and 46% respectively of cells expressing GFP after 24 days.

We conclude that expression of CD28-D124E-T195P, or co-expression of ESR Fas/4-1BB plus Casp7-DN, or co-expression of STAT3-Y640F plus PIK3CA-L1001P in T-cells provides them with a survival or proliferation advantage, and that these genes or gene combinations are immune cell survival-enhancing genes and an immune cell proliferation-enhancing genes as described in Section 5.3.1.1.

### Survivin and Activating Mutants of CD28 Enhance T-Cell Function

We found expression of Survivin or the D124E/T195P activated double mutant of CD28 to enhance T-cell growth/survival and/or proliferation, as shown in Section 6.2.1.2 and Table 5. To test whether these genes could also enhance T-cell performance we integrated them into the genomes of T-cells together with a chimeric antigen receptor targeting CD19, an epitope naturally found exclusively on B-cells, and tested the ability of the modified T-cells to kill cells of a transformed B-cell line.

Three gene transfer polynucleotides were constructed: each comprised a GFP reporter (sequence SEQ ID NO: 222) comprising a gene encoding DasherGFP operably linked to a glyceraldehyde-3-phosphate dehydrogenase (GAPDH) promoter and a bovine growth hormone (BGH) polyadenylation signal sequence. The GFP gene was flanked on one side by an HS4 insulator (sequence SEQ ID NO: 92), and on the other by a D4Z4 insulator (sequence SEQ ID NO: 88). The gene transfer polynucleotide further comprised, on the distal side of one insulator, a target sequence 5′-TTAA-3′, immediately followed by a piggyBac-like transposon inverted terminal repeat sequence SEQ ID NO: 10 (which is an embodiment of SEQ ID NO: 6), immediately followed by additional transposon end sequences with SEQ ID NO: 3 (which is >95% identical to SEQ ID NO: 1). The gene transfer polynucleotide further comprised, on the distal side of the other insulator, additional transposon end sequences with SEQ ID NO: 5 (which is >95% identical to SEQ ID NO: 4), immediately followed by a piggyBac-like transposon inverted terminal repeat sequence SEQ ID NO: 11 (which is an embodiment of SEQ ID NO: 7) immediately followed by a target sequence 5′-TTAA-3′. The gene transfer polynucleotide further comprised a gene encoding a CD10-binding chimeric antigen receptor, a polypeptide with sequence given by SEQ ID NO: 229, operably linked to either a PGK promoter or a GAPDH promoter: promoters that appear comparably active in T-cells as described in Section 6.1.2 and shown in Table 3. The chimeric antigen receptor gene was present in the gene transfer polynucleotide such that it was transcribed divergently from the Dasher GFP gene, and such that it was in the part of the gene transfer polynucleotide that was transposable by the transposase. The first gene transfer polynucleotide (346463 with sequence given by SEQ ID NO: 225 comprised no additional transposable genes. The second gene transfer polynucleotide (346776 with sequence given by SEQ ID NO: 226) further comprised an open reading frame encoding Survivin operably linked to a PGK promoter, transcribed in the same direction as the chimeric antigen receptor and also in the part of the gene transfer polynucleotide that was transposable by the transposase. The third gene transfer polynucleotide (346777 with sequence given by SEQ ID NO: 227) comprised the chimeric antigen receptor and further comprised an open reading frame encoding CD28-D124E-T195P operably linked to a PGK promoter, transcribed in the same direction as the chimeric antigen receptor and also in the part of the gene transfer polynucleotide that was transposable by the transposase.

### Survivin and Activated CD28-Enhanced Ex Vivo CAR Cell Killing Test 1

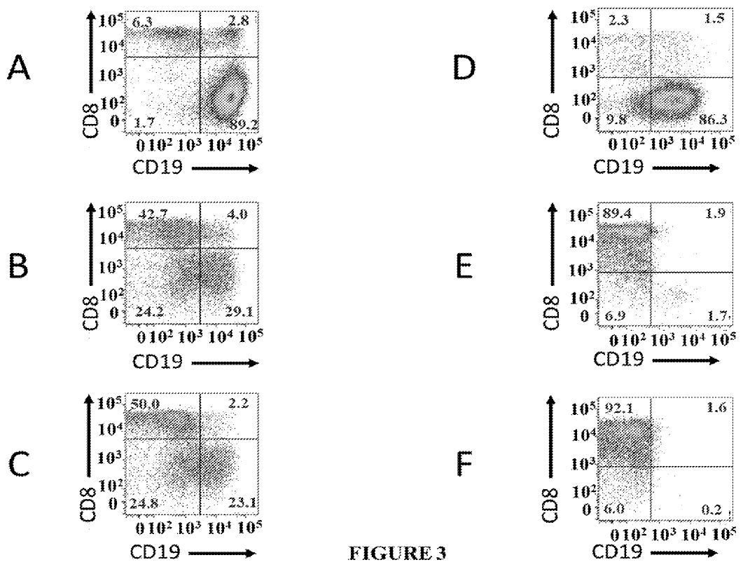

T-cells were prepared from peripheral blood mononuclear cells (PBMCs) from two different normal donors using the EasySep Human CD8 positive selection kit from Stemcell Technologies according the manufacturer's instructions. T-cells were stimulated for 2-3 days by incubation with irradiated feeder cells to provide secreted CD3, CD28, IL-2, IL-7 and IL-15. Approximately 200,000 T-cells were transfected with 1 μg of transposon DNA and 100 ng mRNA encoding transposase with SEQ ID NO: 37 using a Neon electroporator according to the manufacturer's protocol. Transfected T-cells were mixed with feeder cells and incubated at 37° C. Cells were grown in culture for approximately 5 weeks, at which time around 10% of the cells transfected with each gene transfer polynucleotide were expressing GFP. A sample of the T-cells (200,000) were then mixed with an equal number of JY cells: JY is an Epstein-Barr virus-immortalized B-cell lymphoblastoid line that expresses CD19 and is thus a target for the anti-CD19 chimeric antigen receptor. Samples of cells were taken from the cell mixture 3 and 7 days post-mixing, stained with anti-CD8 and anti-CD19 antibodies (to label the T-cells and JY cells respectively). The results are shown in FIG. 3 and Table 6. By the third day post-mixing, T-cells expressing the chimeric antigen receptor alone had largely disappeared, having been overwhelmed by the JY tumor cells: only 8% of the detectable cells were expressing CD8, while 89% were expressing CD19 (FIG. 3 panel A, Table 6 column A rows 3 and 4). By 7 days post-mixing, only 2.3% of the cells were T-cells expressing CD8 (FIG. 3 panel D, Table 6 column A rows 5 and 6). In contrast, T-cells expressing the chimeric receptor plus either Survivin or CD28-D124E-T195P were able to survive in the presence of the JY-tumor cells. After 3 days, 40-50% of the cells were expressing CD8 (the T-cell marker) and only 23-29% were expressing CD19 (the tumor cell marker), as shown in FIG. 3 panels B and C and Table 6 columns B and C, rows 3 and 4. By day 7, the tumor cells had been effectively eliminated, with approximately 90% of all cells expressing CD8 (FIG. 3 panels E and F and Table 6 columns B and C, rows 5 and 6). We conclude that expression of Survivin or the D124E/T195P activated double mutant of CD28 not only enhance T-cell growth/survival and/or proliferation, they can also enhance T-cell performance by enabling T-cells to survive in the presence of tumor cells and remain active to kill the tumor cells.

### Survivin and Activated CD28-Enhanced In Vivo CAR Cell Killing

A second sample of the transfected T-cells were sorted using FACS to select cells expressing GFP (which was an indicator of the presence in the T-cell genome of the transposon). The selected cells were grown in culture for a further week and tested for their ability to kill JY tumor cells in vivo. One million JY cells were administered by intraperitoneal injection to NSG immunocompromised mice. Seven days later, one million GFP-expressing T-cells were administered by intraperitoneal injection to the JY-treated mice. Two mice received an inactive control treatment of phosphate buffered saline (PBS) in place of the T-cells. As shown in Table 7, mice that received the PBS survived for 24 or 25 days after the JY cell injection (Table 7 rows 1 and 2). Administration of T-cells expressing the chimeric antigen receptor extended survival for 5-6 days to 30 days post-JY injection (Table 7 row 3). Administration of T-cells expressing the chimeric antigen receptor plus Survivin or CD28-D124E-T195P extended survival for an additional 4 days to 34 days post-JY injection (Table 7 rows 4 and 5). We conclude that expression of Survivin or the D124E/T195P activated double mutant of CD28 not only enhance T-cell growth/survival and/or proliferation ex-vivo, they can also enhance T-cell performance in vivo by enabling T-cells to survive in the presence of tumor cells and remain active to kill the tumor cells.

### Survivin and Activated CD28-Enhanced Ex Vivo CAR Cell Killing Test 2

We also performed a tumor re-challenge test on T-cells expressing either the anti-CD19 chimeric antigen receptor alone, or the chimeric antigen receptor co-expressed with Survivin or CD28-D124E-T195P. The standard single challenge ex-vivo tumor-lysis assay often over-estimates the true antitumor potential of T-cells due to the relatively short co-culture time and high T-cell to tumor ratio. To determine whether survival-enhancing genes (in this case Survivin and CD28-D124E-T195P) can also enhance T-cell function, we used a recursive high tumor cell load challenge to better mimic the surrounding tumor microenvironment challenging the survival of the T-cells. T-cells (100,000) were challenged with either 1, 2, 3, 4 or 5 consecutive doses of 100,000 NALM6 (which is CD19+, CD20-, CD21-) cells, in a microtiter plate well with a total volume of 200 μl. Each 100,000 cell NALM6 dose was spaced 48 hours apart. For each re-challenge, 100 μl of supernatant was withdrawn, and 100 μl of fresh media containing 100,000 NALM6 cells was added. Twenty-four hours after the last challenge for a sample, NALM6 cell death was measured as a reduction in bioluminescence (see for example Karimi et. al., (2014) Measuring Cytotoxicity by Bioluminescence Imaging Outperforms the Standard Chromium-51 Release Assay. PLoS ONE 9(2): e89357), by addition of D-luciferin and measuring luminescence using a BioTek synergy Neo2 hybrid microplate reader according to the manufacturer's instructions.

Cells transfected with transposons comprising an anti-CD19 CAR and optionally a gene encoding Survivin or a gene encoding activating mutations in CD28, as described in Section 6.2.1.3a, were cultured ex-vivo for 10 months. By this time the cells were >95% GFP-expressing (and by inference also expressing the CAR and, where present, the survival-enhancing genes. It is unusual for T-cells to survive in ex-vivo culture for 10 months. For T-cells expressing the survival genes, we attribute this longevity to expression of Survivin or CD28-D124E-T195P. However, we also observe this long-term survival of T-cells expressing the chimeric antigen receptor alone, although they grew more slowly than the cells also expressing a survival gene. We attribute this to expression of optimal CAR levels when a gene encoding a chimeric antigen receptor is operably linked to a PGK promoter or a GAPDH promoter or a promoter that drives a comparable expression level. In case prolonged ex-vivo culturing had compromised the ability to T-cells to kill tumor cells, we repeated the transfection described in Section 6.2.1.3a into T-cells from a different donor, and cultured the cells ex-vivo for 4 months before testing them with the tumor re-challenge assay. NALM6 cell death results are shown in Table 8.

Table 8 row 1 shows the number of times T-cells were challenged with NALM6 cells. Table 8 rows 2-4 show the NALM6 killing by T-cell populations expressing an anti-CD19 chimeric antigen receptor that were grown ex-vivo for 10 months. T-cells were also expressing either Survivin (row 3) or CD28-D124E-T195P (row 4). Cells expressing the chimeric antigen receptor alone killed 100% of NALM6 cells on the first challenge, but the killing efficiency fell on subsequent challenges: 85% after the second challenge, 47% after the third, 23% after the fourth and only 10% of NALM6 cells were killed after the fifth challenge (see Table 8 row 2). In contrast cells that also expressed Survivin were able to kill 76% of NALM6 cells at the fifth challenge (Table 8 row 3) and cells that also expressed CD28-D124E-T195P were able to kill 82% of NALM6 cells at the fifth challenge (Table 8 row 4). A similar pattern of killing was seen in cells that had been cultured ex-vivo for only 4 months, although the efficiency of killing was generally higher (Table 8 rows 5-7). Cells expressing the chimeric antigen receptor alone killed 100% of NALM6 cells on the first and second challenges, but the killing efficiency fell on subsequent challenges: 51% after the third, 28% after the fourth and 27% of NALM6 cells were killed after the fifth challenge (see Table 8 row 5). In contrast cells that also expressed Survivin were able to kill 90% of NALM6 cells at the fifth challenge (Table 8 row 6) and cells that also expressed CD28-D124E-T195P were able to kill 91% of NALM6 cells at the fifth challenge (Table 8 row 7).

This shows that expression of Survivin or CD28-D124E-T195P by T-cells expressing a chimeric antigen receptor enhances the targeted cell killing by those cells and reduces the rate at which the cells become exhausted. An advantageous T-cell for killing tumor cells comprises a heterologous polynucleotide comprising an expressible Survivin or CD28-D124E-T195P gene.

### Expression of Bcl2 and Bcl6 in Primary T-Cells

An open reading frame encoding Bcl2 and Bcl6 separated by a viral CHYSL (2A) sequence (the sequence of the full open reading frame Bcl2-2A-Bcl6 is given as SEQ ID NO: 272) was operably linked to a PGK promoter with sequence given by SEQ ID NO: 115 and a rabbit globin polyadenylation signal with sequence SEQ ID NO: 182 and cloned into a gene transfer polynucleotide. The gene transfer polynucleotide further comprised a GFP reporter (sequence SEQ ID NO: 222) comprising a gene encoding DasherGFP operably linked to a glyceraldehyde-3-phosphate dehydrogenase (GAPDH) promoter and a bovine growth hormone (BGH) polyadenylation signal sequence. The two open reading frames were configured to be divergently transcribed (i.e. the two promoters were adjacent to each other and transcribed in opposite directions). The two open reading frames were flanked on one side by an HS4 insulator (sequence SEQ ID NO: 92), and on the other by a D4Z4 insulator (sequence SEQ ID NO: 88). The gene transfer polynucleotide further comprised, on the distal side of one insulator, a target sequence 5′-TTAA-3′, immediately followed by a piggyBac-like transposon inverted terminal repeat sequence SEQ ID NO: 10 (which is an embodiment of SEQ ID NO: 6), immediately followed by additional transposon end sequences with SEQ ID NO: 3 (which is >95% identical to SEQ ID NO: 1). The gene transfer polynucleotide further comprised, on the distal side of the other insulator, additional transposon end sequences with SEQ ID NO: 5 (which is >95% identical to SEQ ID NO: 4), immediately followed by a piggyBac-like transposon inverted terminal repeat sequence SEQ ID NO: 11 (which is an embodiment of SEQ ID NO: 7) immediately followed by a target sequence 5′-TTAA-3′.

T-cells were prepared from normal donor peripheral blood mononuclear cells (PBMCs) using the EasySep Human CD8 positive selection kit from Stemcell Technologies according the manufacturer's instructions. T-cells were stimulated for 2-3 days by incubation with irradiated feeder cells to provide secreted CD3, CD28, IL-2, IL-7 and IL-15. Approximately 100,000 T-cells were transfected with 1 μg transposon DNA and 100 ng mRNA encoding transposase with sequence SEQ ID NO: 37 using a Neon electroporator according to the manufacturer's protocol. Transfected T-cells were mixed with feeder cells and incubated at 37° C. Samples were taken at various times post-transfection, incubated with a fluorescently-labelled anti-CD8 antibody, and analyzed on a fluorescence-activated cell sorter (FACS) for CD8 and Dasher GFP.

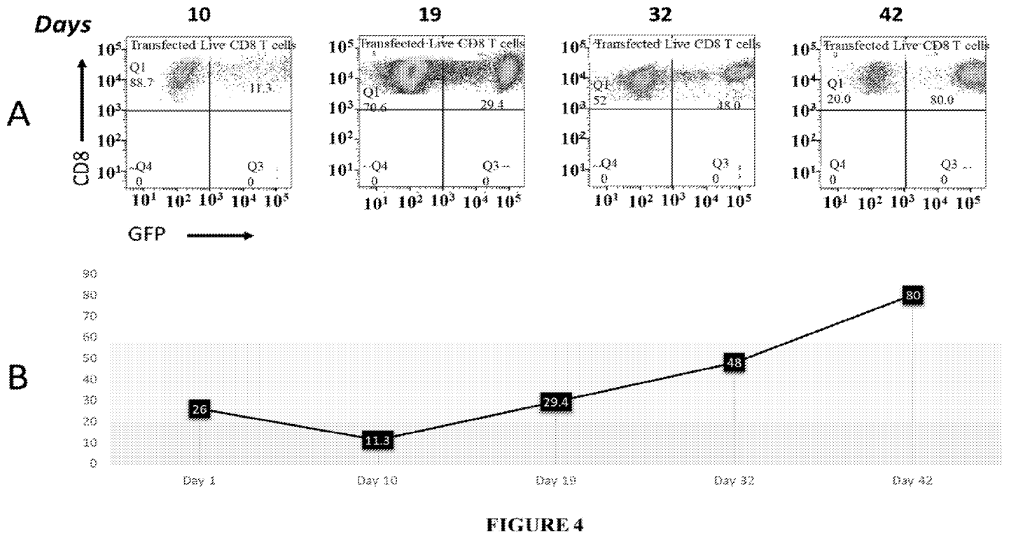

FIG. 4 shows the distribution of cell staining over time. CD8 staining is used as a marker for CD8+ T-cells, and is shown on the y-axis of each of the FACS plots shown in Panel A. GFP fluorescence is shown on the x-axis of each FACS plot; GFP fluorescence indicates that the cell is expressing GFP, it is also used here as a marker to indicate the presence of the gene transfer polynucleotide within the cell. Panel B is a graph showing the percentage of CD8-expressing T-cells that were also expressing GFP. On the first day post-transfection, approximately 26% of CD8-expressing cells were also expressing GFP. By day 10, 88.7% of the cells showed strong CD8-staining but no GFP expression, 11.3% of the CD8-expressing cells also expressed GFP, indicating that they also contained the gene transfer polynucleotide. The fraction of CD8-expressing cells also exhibiting GFP fluorescence increased over time: 29.4% at day 19, 80% at day 42. The increase in the fraction of the T-cell population expressing GFP either indicates that the T-cells whose genomes comprise the gene transfer polynucleotide possess a survival advantage compared with the T-cells whose genomes do not comprise the gene transfer polynucleotide, or it indicates that the T-cells whose genomes comprise the gene transfer polynucleotide possess a proliferation advantage compared with the T-cells whose genomes do not comprise the gene transfer polynucleotide. We conclude that expression of Bcl2 and Bcl6 in T-cells provides them with a survival or proliferation advantage, and that a gene encoding Bcl2 and Bcl6 is an immune cell survival-enhancing gene and an immune cell proliferation-enhancing gene as described in Section 5.3.1.1.

### Expression of T-Cell Transformation Elements and ESRs in Primary T-Cells

Genes encoding a set of T-cell transformation elements and enhanced signaling receptors were cloned individually into separate gene transfer polynucleotides. In each case the gene was operably linked to a PGK promoter with sequence given by SEQ ID NO: 115 and a rabbit globin polyadenylation signal with sequence SEQ ID NO: 182. The gene transfer polynucleotide further comprised a GFP reporter (sequence SEQ ID NO: 222) comprising a gene encoding DasherGFP operably linked to a glyceraldehyde-3-phosphate dehydrogenase (GAPDH) promoter and a bovine growth hormone (BGH) polyadenylation signal sequence. The two open reading frames were configured to be divergently transcribed (i.e. the two promoters were adjacent to each other and transcribed in opposite directions). The two open reading frames were flanked on one side by an HS4 insulator (sequence SEQ ID NO: 92), and on the other by a D4Z4 insulator (sequence SEQ ID NO: 88). The gene transfer polynucleotide further comprised, on the distal side of one insulator, a target sequence 5′-TTAA-3′, immediately followed by a piggyBac-like transposon inverted terminal repeat sequence SEQ ID NO: 10 (which is an embodiment of SEQ ID NO: 6), immediately followed by additional transposon end sequences SEQ ID NO: 3 (which is >95% identical to SEQ ID NO: 1). The gene transfer polynucleotide further comprised, on the distal side of the other insulator, additional transposon end sequences SEQ ID NO: 5 (which is >95% identical to SEQ ID NO: 4), immediately followed by a piggyBac-like transposon inverted terminal repeat sequence SEQ ID NO: 11 (which is an embodiment of SEQ ID NO: 7) immediately followed by a target sequence 5′-TTAA-3′.

T-cells were prepared from the peripheral blood mononuclear cells (PBMCs) of two donors using the EasySep Human CD8 positive selection kit from Stemcell Technologies according to the manufacturer's instructions. T-cells were stimulated for 2-3 days by incubation with irradiated feeder cells to provide secreted CD3, CD28, IL-2, IL-7 and IL-15. Approximately 100,000 T-cells were transfected with 1 μg transposon DNA and 100 ng mRNA encoding transposase with SEQ ID NO: 37 using a Neon electroporator according to the manufacturer's instructions. Transfected T-cells were mixed with feeder cells and incubated at 37° C. Samples were taken at various times post-transfection, incubated with a fluorescently-labelled anti-CD8 antibody, and analyzed on a fluorescence-activated cell sorter (FACS) for CD8 and Dasher GFP. The data is shown in Table 9.

As described in Section 6.2.1.1, the enrichment of CD8+ cells expressing GFP is an indicator that the gene transfer polynucleotide comprises a gene that confers a survival or a proliferation advantage to a T-cell, as described in Section 5.3.1.1. In this set of gene transfer polynucleotides, HSV-TK was included as a control gene that is expected to have no effect on T-cell survival. We therefore used the percentage of cells expressing GFP in cells transfected with HSV-TK as a level against which to benchmark the putative survival-enhancing genes. As seen in Table 9, two independent transfections of T-cells from 2 donors resulted in initial GFP expression (indicating transfection efficiency) in between 7.5% and 15.3% of cells (Table 9 rows 7 and 8). By day 14 these percentages had fallen significantly, and at subsequent times the percentage of cells expressing GFP remained approximately steady or declined. This indicates that, as expected, HSV-TK does not provide T-cells with a growth or proliferation advantage. In contrast, two of the tested gene transfer polynucleotides comprising genes encoding mutants of STAT3: STAT3-D661Y and STAT3-S614R-Y640F showed a progressive increase in the percentage of cells expressing GFP in both donors (Table 9 rows 1 and 3), indicating that these genes do provide T-cells with a growth or proliferation advantage, similar to that seen for STA3-Y640F in Section 6.2.1.1. We conclude that expression of activating STAT3 mutants including STAT3-D661Y and STA3-S614R-Y640F in T-cells provides them with a survival or proliferation advantage, and that a gene encoding an activating mutant of STAT3 is an immune cell survival-enhancing gene and an immune cell proliferation-enhancing gene as described in Section 5.3.1.1.

One of the tested gene transfer polynucleotides comprised a gene encoding the inhibitor of apoptosis Bcl-XL. These cells showed a progressive increase in the percentage of cells expressing GFP in both donors (Table 9 row 2), indicating that expression of Bcl-XL provides T-cells with a growth or proliferation advantage, and that a gene encoding Bcl-XL is an immune cell survival-enhancing gene and an immune cell proliferation-enhancing gene as described in Section 5.3.1.1.

One of the tested gene transfer polynucleotides comprised a gene encoding an activating mutation of phospholipase C: PLCG1-S345F. These cells showed an increase in the percentage of cells expressing GFP in both donors (Table 9 row 6), indicating that expression of PLCG1-S345F provides T-cells with a growth or proliferation advantage, and that a gene encoding PLCG1-S345F is an immune cell survival-enhancing gene and an immune cell proliferation-enhancing gene as described in Section 5.3.1.1.

Two ESRs were also tested in this experiment. One TNFR1/CD27 (with sequence given by SEQ ID NO: 301) comprised an extracellular domain from TNFRSF1A (with sequence given by SEQ ID NO: 330), a transmembrane domain from TNFRSF1A (with sequence given by SEQ ID NO: 394) and an intracellular domain from CD27 (with sequence given by SEQ ID NO: 343). A second TNFR1/4-1BB (with sequence given by SEQ ID NO: 302) also comprised an extracellular domain from TNFRSF1A (with sequence given by SEQ ID NO: 330) and a transmembrane domain from TNFRSF1A (with sequence given by SEQ ID NO: 394) in this case fused to an intracellular domain from 4-1BB (with sequence given by SEQ ID NO: 344). ESR TNFR1/CD27 and ESR TNFR1/4-1BB both resulted in high percentages of cells expressing GFP in one of the donors (Table 9 row 4) indicating that expression of ESR TNFR1/CD27 or ESR TNFR1/4-1BB can provide T-cells with a growth or proliferation advantage, and that a gene encoding ESR TNFR1/CD27 or ESR TNFR1/4-1BB is an immune cell survival-enhancing gene and an immune cell proliferation-enhancing gene as described in Section 5.3.1.1.

### Effect of Bel-XL on Tumor Cell Killing by Primary T-Cells

### Bcl-XL-Enhanced Ex Vivo BiTE Cell Killing Test

A gene encoding Bcl-XL (with polypeptide sequence given by SEQ ID NO: 238) was cloned into a gene transfer polynucleotide. The gene was operably linked to a PGK promoter with sequence given by SEQ ID NO: 115 and a rabbit globin polyadenylation signal with sequence SEQ ID NO: 182. The gene transfer polynucleotide further comprised a GFP reporter (sequence SEQ ID NO: 222) comprising a gene encoding DasherGFP operably linked to a glyceraldehyde-3-phosphate dehydrogenase (GAPDH) promoter and a bovine growth hormone (BGH) polyadenylation signal sequence. The two open reading frames were configured to be divergently transcribed (i.e. the two promoters were adjacent to each other and transcribed in opposite directions). The two open reading frames were flanked on one side by an HS4 insulator (with sequence SEQ ID NO: 92), and on the other by a D4Z4 insulator (with sequence SEQ ID NO: 88). The gene transfer polynucleotide further comprised, on the distal side of one insulator, a target sequence 5′-TTAA-3′, immediately followed by a piggyBac-like transposon inverted terminal repeat sequence SEQ ID NO: 10 (which is an embodiment of SEQ ID NO: 6), immediately followed by additional transposon end sequences SEQ ID NO: 3 (which is >95% identical to SEQ ID NO: 1). The gene transfer polynucleotide further comprised, on the distal side of the other insulator, additional transposon end sequences SEQ ID NO: 5 (which is >95% identical to SEQ ID NO: 4), immediately followed by a piggyBac-like transposon inverted terminal repeat sequence SEQ ID NO: 11 (which is an embodiment of SEQ ID NO: 7) immediately followed by a target sequence 5′-TTAA-3′.

T-cells were prepared from the peripheral blood mononuclear cells (PBMCs) of three donors using the EasySep Human CD8 positive selection kit from Stemcell Technologies according the manufacturer's instructions. T-cells were stimulated for 2-3 days by incubation with irradiated feeder cells to provide secreted CD3, CD28, IL-2, IL-7 and IL-15. Approximately 100,000 T-cells were transfected with 1 μg transposon DNA and 100 ng mRNA encoding transposase with SEQ ID NO: 37 using a Neon electroporator according to the manufacturer's protocol. Transfected T-cells were mixed with feeder cells and incubated at 37° C.

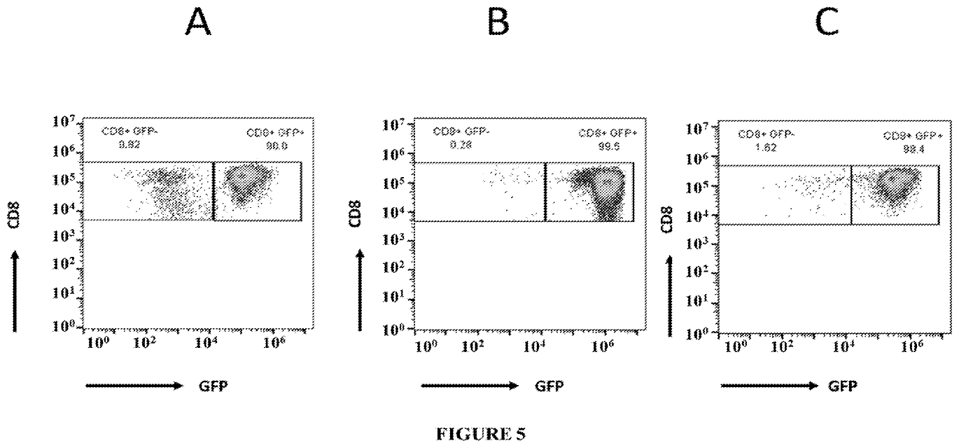

Cells were grown in culture in T-cell media for 240 days. As described in Section 6.2.1.6, and in Table 9, Bcl-XL provides a selective advantage to T-cells. That we were able to culture these cells for 8 months demonstrates that the expression of the Bcl-XL gene in T-cells enhances their survival ex-vivo. In addition to survival, we tested whether these T-cells retained their cytotoxicity by mixing them with a tumor cell line. Prior to testing cytotoxicity, we determined the fraction of cells expressing Bcl_X1 by measuring GFP which is expressed from the same transposon integrated into the T-cell genome. FIG. 5 shows flow cytometry analysis of the T-cells from the three different donors 240 days after they were transfected, with GFP on the x-axis and staining for the T-cell marker CD8 on the y-axis. Panel A shows that over 90% of T-cells from Donor 81 expressed GFP, Panel B B shows that over 99% of T-cells from Donor 82 expressed GFP, Panel C shows that over 98% of T-cells from Donor 84 expressed GFP. Thus after 240 days the great majority of T-cells from each of the donors were expressing GFP, and by inference Bcl-XL.

To measure cytotoxicity, we mixed 100,000 T-cells with 100,000 cells of a B-cell tumor line, NALM6 (which is CD19+, CD20-, CD21-) containing a genomically integrated gene encoding luciferase. A Bi-specific T-cell engager (BiTE) with binding domains against CD3 (on the T-cell surface) and CD19 (on the NALM6 surface) was included in some reactions to bring the T-cell to the tumor target cell. The following day we used a bioluminescence assay (see for example Karimi et. al., (2014) Measuring Cytotoxicity by Bioluminescence Imaging Outperforms the Standard Chromium-51 Release Assay. PLoS ONE 9(2): e89357) to determine the fraction of NALM6 cells that had been lysed.

The cytotoxicity test was performed as a tumor re-challenge. The standard single challenge ex-vivo tumor-lysis assay often over-estimates the true antitumor potential of T-cells due to the relatively short co-culture time and high T-cell to tumor ratio. To determine whether a survival-enhancing gene (in this case Bcl-XL) can also enhance T-cell function, we used a recursive high tumor cell load challenge to better mimic the surrounding tumor microenvironment challenging the survival of the T-cells. T-cells (100,000) were challenged with either 1, 2, 3, 4 or 5 consecutive doses of 100,000 NALM6 cells, in a microtiter plate well with a total volume of 200 μl. Each 100,000 cell NALM6 dose was spaced 48 hours apart. For each re-challenge, 100 μl of supernatant was withdrawn, and 100 μl of fresh media containing 100,000 NALM6 cells was added. Twenty-four hours after the last challenge for a sample, NALM6 cell death was measured as a reduction in bioluminescence, by addition of D-luciferin and measuring luminescence using a BioTek synergy Neo2 hybrid microplate reader according to the manufacturer's instructions. NALM6 cell death results are shown in Table 10.

Table 10 row 1 shows the number of times T-cells were challenged with NALM6 cells. Table 10 rows 2-5 show the NALM6 killing by four different T-cell populations in the absence of any BiTE. Cell killing under these conditions reflects general allogenic killing, there is no specific targeting to a tumor antigen. The killing achieved by the three T-cell populations expressing Bcl-XL (Table 10, rows 2-4) was very comparable to the killing achieved by naïve T-cells (Table 10 row 5). Of note is the fact that the naïve T-cells were cultured for only a few weeks prior to their use in this experiment, in contrast to the Bcl-XL-expressing T-cells which had been cultured for 8 months. This shows that Bcl-XL expression can allow T-cells to grow in culture for 8 months while retaining their cytotoxicity.

A second set of challenges were performed in the presence of a BiTE which targets the CD19 antigen on the surface of the NALM6 cells. Table 10 row 9 shows the NALM6 killing effected by naïve T-cells in the presence of the BiTE. Killing after the first and second challenge was much more efficient than without the BiTE: 88% of NALM6 cells were killed on the first challenge, and 97% were killed after the second challenge. The efficiency of killing then decreased: 72% of the NALM6 cells were killed after the third challenge, 62% after the fourth challenge and 59% after the fifth challenge. This decrease is emblematic of loss of T-cell efficacy seen after long-term tumor re-challenge (see for example Voss et., al. (2017) Cancer Lett. 408: 190-196. “Metabolic reprogramming and apoptosis sensitivity: defining the contours of a T cell response”). The three T-cell populations expressing Bcl-XL were as effective at killing NALM6 after 1 or 2 challenges in the presence of the BiTE as were the naïve T-cells (Table 10 rows 6-8). Unlike naïve T-cells, the NALM6-killing efficiency of Bcl-XL-expressing T-cells did not decrease upon successive challenges. After the fifth challenge, two of the Bcl-XL-expressing T-cell populations (from donors 81 and 84) killed 95% of the NALM6 cells (Table 10 rows 6 and 8), and the third population (from donor 82) killed 94% of the NALM6 cells (Table 10 row 7), compared with 59% killing for the naïve T-cells. This shows that not only are T-cells expressing Bcl-XL that have been grown ex-vivo for 8 months still as capable of killing tumor cells as are naïve T-cells that have only been cultured for a few weeks; they also appear to be less susceptible to factors that reduce T-cell efficacy after repeated exposure to tumor antigen.

### Bcl-XL-Enhanced Ex Vivo CAR Cell Killing Test

We performed a tumor re-challenge test on T-cells expressing the anti-CD19 chimeric antigen receptor as described in Section 6.2.1.3c. Transposons were as described in Section 6.2.1.3 comprising the chimeric antigen receptor co-expressed with Survivin or CD28-D124E-T195P, and an additional transposon was made that comprised the chimeric antigen receptor co-expressed with Bcl-XL. To determine whether survival-enhancing genes (in this case Survivin, CD28-D124E-T195P and Bcl-XL) can also enhance T-cell function, we used a recursive high tumor cell load challenge to better mimic the surrounding tumor microenvironment challenging the survival of the T-cells. T-cells (100,000) were challenged with either 1, 2, 3, 4, 5 or 6 consecutive doses of 100,000 NALM6 (which is CD19+, CD20-, CD21-) cells, in a microtiter plate well with a total volume of 200 μl. Each 100,000 cell NALM6 dose was spaced 48 hours apart. For each re-challenge, 100 μl of supernatant was withdrawn, and 100 μl of fresh media containing 100,000 NALM6 cells was added. Twenty-four hours after the last challenge for a sample, NALM6 cell death was measured as a reduction in bioluminescence (see for example Karimi et. al., (2014) Measuring Cytotoxicity by Bioluminescence Imaging Outperforms the Standard Chromium-51 Release Assay, PLoS ONE 9(2): e89357), by addition of D-luciferin and measuring luminescence using a BioTek synergy Neo2 hybrid microplate reader according to the manufacturer's instructions.

Cells transfected with transposons comprising an anti-CD19 CAR and optionally a gene encoding Survivin or a gene encoding activating mutations in CD28, or a gene encoding Bcl-XL, were cultured ex-vivo for 4 months. By this time the cells were >95% GFP-expressing (and by inference also expressing the CAR and, where present, the survival-enhancing genes. NALM6 cell death results are shown in Table 11.

Table 11 row 1 shows the number of times T-cells were challenged with NALM6 cells. Table 11 row 2 shows the NALM6 killing by T-cell populations expressing an anti-CD19 chimeric antigen receptor. T-cells were also expressing either Survivin (row 3), CD28-D124E-T195P (row 4), or Bcl-XL (row 5). Cells with no chimeric antigen receptor are shown in row 6. Cells expressing the chimeric antigen receptor alone killed 70% of NALM6 cells on the first challenge, the killing efficiency rose to 97% on the second challenge, but then fell again on subsequent challenges: 69% after the third challenge, 59% after the fourth, 58% on the fifth and 53% of NALM6 cells were killed after the sixth challenge (see Table 11 row 2). In contrast, cells that also expressed Survivin were able to kill 85% of NALM6 cells at the sixth challenge (Table 11 row 3); cells that also expressed CD28-D124E-T195P were able to kill 85% of NALM6 cells at the sixth challenge (Table 11 row 4) and cells that also expressed Bcl-XL were able to kill 94% of NALM6 cells at the sixth challenge (Table 11 row 5). This shows that expression of Survivin or CD28-D124E-T195P or Bcl-XL by T-cells expressing a chimeric antigen receptor enhances the targeted cell killing by those cells and reduces the rate at which the cells become unable to kill tumor cells. An advantageous T-cell for killing tumor cells comprises a gene encoding a chimeric antigen receptor and a heterologous polynucleotide comprising an expressible Survivin or CD28-D124E-T195P or Bcl-XL gene.

Inhibitors of apoptosis Survivin, Bcl-XL, Bcl2 and Bcl6 are all shown here to act as immune cell survival genes. Dominant negative gene in the caspase pathway for example a dominant negative mutant of Caspase 3, Caspase 7, Caspase 8, Caspase 9, Caspase 10 or CASP8 and FADD-like apoptosis regulator (CFLAR) are anticipated to have similar effects. In some embodiments of the invention, an immune cell comprises a gene encoding a dominant negative inhibitor of the apoptotic pathway comprising a dominant negative mutant of a sequence selected from among SEQ ID NO: 240-245; in some embodiments the inhibitor of the apoptotic pathway comprises a sequence selected from among SEQ ID NO: 237, 238 or 261-272.

### Enhanced Signaling Receptors

Anti-CD28/OX40 is an ESR with Proliferation-Enhancing Activity

A gene was designed to encode an anti-CD28/OX40 ESR (with sequence given by SEQ ID NO: 307) comprising anti-CD28 antibody TGN1412 (with sequence given by SEQ ID NO: 340) fused to the transmembrane domain for TNFRSF4 (OX40) (with sequence given by SEQ ID NO: 373) and the intracellular domain for TNFRSF4 (OX40) (with sequence given by SEQ ID NO: 341). The gene was operably linked to a PGK promoter with sequence given by SEQ ID NO: 115 and a rabbit globin polyadenylation signal sequence with SEQ ID NO: 182. The gene transfer polynucleotide further comprised a GFP reporter (with sequence SEQ ID NO: 222) comprising a gene encoding DasherGFP operably linked to a glyceraldehyde-3-phosphate dehydrogenase (GAPDH) promoter and a bovine growth hormone (BGH) polyadenylation signal sequence. The two open reading frames were configured to be divergently transcribed (the two promoters were adjacent to each other and transcribed in opposite directions). The two open reading frames were flanked on one side by an HS4 insulator (with sequence SEQ ID NO: 92), and on the other by a D4Z4 insulator (with sequence SEQ ID NO: 88). The gene transfer polynucleotide further comprised, on the distal side of one insulator, a target sequence 5′-TTAA-3′, immediately followed by a piggyBac-like transposon inverted terminal repeat sequence SEQ ID NO: 10 (which is an embodiment of SEQ ID NO: 6), immediately followed by additional transposon end sequences SEQ ID NO: 3 (which is >95% identical to SEQ ID NO: 1). The gene transfer polynucleotide further comprised, on the distal side of the other insulator, additional transposon end sequences SEQ ID NO: 5 (which is >95% identical to SEQ ID NO: 4), immediately followed by a piggyBac-like transposon inverted terminal repeat sequence SEQ ID NO: 11 (which is an embodiment of SEQ ID NO: 7) immediately followed by a target sequence 5′-TTAA-3′.

T-cells were prepared from peripheral blood mononuclear cells (PBMCs) from two different normal donors using the EasySep Human CD8 positive selection kit from Stemcell Technologies according to the manufacturer's instructions. T-cells were stimulated for 2-3 days by incubation with irradiated feeder cells to provide secreted CD3, CD28, IL-2, IL-7 and IL-15. Approximately 100,000 T-cells were transfected with 1 μg transposon DNA and 100 ng mRNA encoding transposase with sequence given by SEQ ID NO: 37 using a Neon electroporator according to the manufacturer's protocol. Transfected T-cells were mixed with feeder cells and incubated at 37° C. Samples were taken at 1, 14 and 28 days post-transfection, incubated with a fluorescently-labelled anti-CD8 antibody, and analyzed on a fluorescence-activated cell sorter (FACS) for CD8 and Dasher GFP. The data is shown in Table 12.

As described in Section 6.2.1.1, the enrichment of CD8+ cells expressing GFP is an indicator that the gene transfer polynucleotide comprises a gene that confers a survival or a proliferation advantage to a T-cell, as described in Section 5.3.1.1. T-cells transfected with the anti-CD28/OX40 ESR gene showed extremely rapid accumulation of GFP. In cells from one donor 94% of CD8+ cells were GFP+ within 14 days. In cells from a second donor, 98% of cells were GFP+ within 28 days. In contrast cells transfected with a control gene, HSV-TK showed comparable initial (day 1) GFP levels, but these levels decreased rather than the GFP expressing cells becoming enriched. This data indicates that expression of the anti-CD28/OX40 ESR provided a very significant growth/proliferation advantage to T-cells that express the ESR.

### ESR FAS/4-1BB Stimulates Proliferation in the Presence of Casp7-DN

A gene was designed to encode an ESR comprising the extracellular domain of TNFRSF6 (Fas) (with sequence given by SEQ ID NO: 323), and further comprising the transmembrane domain of TNFRSF6 (Fas) (with sequence given by SEQ ID NO: 387) and further comprising the intracellular domain of TNFRSF9 (4-1BB) (with sequence given by SEQ ID NO: 344). This ESR (Fas/4-1BB) comprised sequence SEQ ID NO: 274. A second gene encoding an inhibitor of apoptosis: a dominant negative version of Casp7: Casp7-DN (with sequence given by SEQ ID NO: 262) was also designed.

The ESR and Casp7-DN were separately cloned into a transposon-based gene transfer vector. Each gene was operably linked to a PGK promoter with sequence given by SEQ ID NO: 115 and a rabbit globin polyadenylation signal sequence with sequence given by SEQ ID NO: 182. Each gene transfer polynucleotide further comprised a GFP reporter (with sequence SEQ ID NO: 222) comprising a gene encoding DasherGFP operably linked to a glyceraldehyde-3-phosphate dehydrogenase (GAPDH) promoter and a bovine growth hormone (BGH) polyadenylation signal sequence. The two open reading frames were configured to be divergently transcribed (the two promoters were adjacent to each other and transcribed in opposite directions). The two open reading frames were flanked on one side by an HS4 insulator (with sequence SEQ ID NO: 92), and on the other by a D4Z4 insulator (with sequence SEQ ID NO: 88). The gene transfer polynucleotide further comprised, on the distal side of one insulator, a target sequence 5′-TTAA-3′, immediately followed by a piggyBac-like transposon inverted terminal repeat sequence SEQ ID NO: 10 (which is an embodiment of SEQ ID NO: 6), immediately followed by additional transposon end sequences SEQ ID NO: 3 (which is >95% identical to SEQ ID NO: 1). The gene transfer polynucleotide further comprised, on the distal side of the other insulator, additional transposon end sequences SEQ ID NO: 5 (which is >95% identical to SEQ ID NO: 4), immediately followed by a piggyBac-like transposon inverted terminal repeat sequence SEQ ID NO: 11 (which is an embodiment of SEQ ID NO: 7) immediately followed by a target sequence 5′-TTAA-3′.

T-cells were prepared from peripheral blood mononuclear cells (PBMCs) from two different normal donors using the EasySep Human CD8 positive selection kit from Stemcell Technologies according to the manufacturer's instructions. T-cells were stimulated for 2-3 days by incubation with irradiated feeder cells to provide secreted CD3, CD28, IL-2, IL-7 and IL-15. Approximately 100,000 T-cells were transfected with 0.5 μg of each transposon DNA and 100 ng mRNA encoding transposase with sequence given by SEQ ID NO: 37 using a Neon electroporator according to the manufacturer's protocol. Transfected T-cells were mixed with feeder cells and incubated at 37° C. Samples were taken at 1, 7, 28, 48 and 54 days post-transfection, incubated with a fluorescently-labelled anti-CD8 antibody, and analyzed on a fluorescence-activated cell sorter (FACS) for CD8 and Dasher GFP. The data is shown in Table 13.

As described in Section 6.2.1.1, the enrichment of CD8+ cells expressing GFP is an indicator that the gene transfer polynucleotide comprises a gene that confers a survival or a proliferation advantage to a T-cell, as described in Section 5.3.1.1. T-cells co-transfected with genes encoding the ESR FAS/4-1BB plus Casp7-DN gene showed an increase in the percentage of cells expressing GFP over time. One day post-transfection 4.5% of CD8+ cells also expressed GFP. After 7 days, 1.9% of the CD8+ cells were expressing GFP, indicating that less than 2% of the CD8+ T-cells had integrated the gene transfer polynucleotides into their nuclei. By 28 days post-transfection, 31% of CD8+ cells were also expressing GFP, indicating that the genes on the gene transfer polynucleotide were enabling the recipient cells to either survive better or to proliferate more rapidly. By 48 days post-transfection, 50.4% of CD8+ cells were also expressing GFP, and by 54 days post-transfection over 97% of the CD8+ T-cells were also expressing GFP. This data indicates that expression of the ESR FAS/4-1BB plus the anti-apoptotic gene Casp7-DN provided a significant survival/proliferation advantage.

### BRIEF DESCRIPTION OF TABLES

Table 1. Xenopus and Bombyx piggyBac-Like Transposons in the Human Jurkat T-Cell Line

Gene transfer polynucleotides with sequence given by SEQ ID NOs: 223 and 224 comprising piggyBac-like transposons were constructed as described in Section 6.1.1. Jurkat cells (200,000 cells per transfection) were transfected with 1 μg of plasmid DNA and 100 ng of transposase mRNA encoding the corresponding transposase, using a Neon electroporator according to the manufacturer's instructions. After various times (shown in column A), cells were labeled with an anti-CD19 antibody and the population of cells were analyzed by FACS to determine the percentage of the cell population expressing CD19. Column B shows the percentage for the Xenopus transposon contained within gene transfer polynucleotide SEQ ID NO: 223; column C shows the percentage for the Bombyx transposon contained within gene transfer polynucleotide with sequence given by SEQ ID NO: 224.

Table 2. Duration of Heterologous Promoter Activity in Jurkat Cells

Plasmids comprising the promoter named in column A, with sequence given by the SEQ ID NO shown in column B, and optionally an intron with sequence given by the SEQ ID NO shown in column C, and further comprising a polynucleotide with sequence given by SEQ ID NO: 218 to the 5′ and a polynucleotide with sequence given by SEQ ID NO: 219 to the 3′ of the promoter were transfected into Jurkat cells as described in Section 6.1.2.1. Cells were fluorescently labeled with an anti-CD19 antibody and analyzed by flow cytometry at various times after transfection. The percentage of cells expressing CD19 on their surfaces are shown after 2 days (column D), 8 days (column E), 16 days (column F) and 23 days (column G). Column H shows the percentage decline in CD19-expressing cells between day 2 and day 23. The same promoters and introns were also operably linked to a gene encoding GFP and transiently transfected into human embryonic kidney (HEK) cells in triplicate. Cells were counted on a fluorimeter 48 hours post-transfection. The mean fluorescence intensity from the three readings is shown in column I.

Table 3. Heterologous Promoter Activity in Jurkat Cells

Plasmids comprising the promoter named in column A, with sequence given by the SEQ ID NO shown in column B, and optionally an intron with sequence given by the SEQ ID NO shown in column C, and further comprising a polynucleotide with sequence given by SEQ ID NO: 218 to the 5′ and a polynucleotide with sequence given by SEQ ID NO: 219 to the 3′ of the promoter were transfected into Jurkat cells as described in Section 6.1.2.1. Eight days later cells were fluorescently labeled with an anti-CD19 antibody and analyzed by flow cytometry. Column D shows the mean fluorescent intensity. Column E shows the calculated average number of CD19 molecules on the surface of the Jurkat cells.

Table 4. Heterologous Promoter Activity in Primary T-Cells

Plasmids comprising the promoter named in column A, with sequence given by SEQ ID NO shown in column B, further comprising a polynucleotide with sequence given by SEQ ID NO: 218 to the 5′ and a polynucleotide with sequence given by SEQ ID NO: 219 to the 3′ of the promoter were transfected into primary T-cells as described in Section 6.1.2.2. Eleven days later cells were fluorescently labeled with an anti-CD19 antibody and analyzed by flow cytometry. Column C shows the mean fluorescent intensity.

Table 5. Enhanced Survival of Primary T-Cells

Gene transfer polynucleotides comprising piggyBac-like transposons were constructed as described in Section 6.2.1.2. Each transposon comprised one putative survival-enhancing gene. Fifteen samples of human primary T-cells were prepared by co-transfection of 1 μg DNA of a single transposon and 100 ng mRNA encoding transposase with sequence given by SEQ ID NO: 37 (rows 1-15). The name of the gene is given in column A, and the SEQ ID NO of the gene is given in column B. Eight samples of human primary T-cells were prepared by co-transfection of 0.5 μg DNA of two different transposons (differing only in the sequence of the putative survival-enhancing gene) and 100 ng mRNA encoding transposase with sequence given by SEQ ID NO: 37 (rows 16-23). The name of the first gene is given in column A, the SEQ ID NO of the first gene is given in column B, the name of the second gene is given in column C, and the SEQ ID NO of the second gene is given in column D. Cells were cultured for 24 days before being analyzed by FACS for the presence of CD8 as a T-cell marker, and the expression of GFP as an indicator of the presence of the gene transfer polynucleotide in the genome of the T-cell. Column E shows the percentage of analyzed cells that were lymphocytes, column F shows the percentage of analyzed cells that were alive, column G shows the percentage of live cells that expressed CD8 on their surface, and column H shows the percentage of CD8+ cells that were expressing GFP.

Table 6. Ex-Vivo Anti-Tumor Activity of Primary T-Cells Expressing Survivin and CD28-D124E-T195P

A gene transfer polynucleotide encoding an anti-CD19 chimeric antigen receptor was constructed on a piggyBac-like transposon as described in Section 6.2.1.3. Human primary T-cells were co-transfected with a transposase and one of three corresponding transposons comprising a gene encoding a chimeric antigen receptor with sequence given by SEQ ID NO: 229 and a GFP reporter as described in Section 6.2.1.3. One transposon comprised no further genes (column A), one transposon further comprised a gene encoding Survivin (column B) and one transposon further comprised a gene encoding CD28-D124E-T195P (column C). Sequences of the gene transfer polynucleotides are given as the SEQ ID NOs shown in row 1. Cells were cultured for approximately 5 weeks, at which point the percentage of the T-cells expressing GFP were measured using FACS (row 2). At that point 200,000 T-cells (˜20,000 GFP-expressing T-cells) were mixed with 200,000 cells of the JY transformed B-cell line. Three days (rows 3 and 4) or 7 days (rows 5 and 6) post-mixing, cells were labelled with fluorescently-labelled anti-CD8 and anti-CD19 antibodies and analyzed using a fluorescence-activated cell sorter. The percentage of cells expressing CD8 is shown in rows 4 and 6, the percentage of cells expressing CD19 is shown in rows 3 and 5.

Table 7. In Vivo Anti-Tumor Activity of Primary T-Cells Expressing Survivin and CD28-D124E-T195P

Human primary T-cells were co-transfected with a transposase and one of three corresponding gene transfer polynucleotides comprising transposons constructed as described in Section 6.2.1.3 and Table 6. Names of the transposons are shown in column A, sequences of the gene transfer polynucleotides are given as the SEQ ID NOs shown in column B. Cells were cultured after transfection for approximately 5 weeks, and then sorted using FACS to select cells expressing GFP which was an indicator of the presence in the T-cell genome of the transposon. The selected cells were grown in culture for a further week and then 1 million cells were administered by intraperitoneal injection to mice that had received an intraperitoneal injection of 1 million JY cells 7 days previously. The length of time (in days) that the mice lived after administration of the JY cells is shown in column C. Rows 1 and 2 received no T-cells, but instead control injections of phosphate buffered saline (PBS).

Table 8. Enhanced Activity of Primary T-Cells Expressing Survivin and CD28-D124E-T195P

Gene transfer polynucleotides comprising piggyBac-like transposons were constructed and transfected into T-cells from 2 different donors as described in Section 6.2.1.3. Cells from one donor were cultured ex-vivo for 10 months, cells from the second donor were cultured ex-vivo for 4 months. GFP-expressing CD8+ T-cells were sorted by FACS then challenged with a NALM6 B-cell tumor line, as described in Section 6.2.1.3. Column A shows the ex-vivo culture time, column B shows whether the cells were expressing a Survivin gene encoded on a heterologous polynucleotide, column C shows whether the cells were expressing a CD28-D124E-T195P gene encoded on a heterologous polynucleotide. Columns D-H show the % of NALM6 killing observed using a luminescence assay. Column D: cells challenged with NALM6 on day 0 and killing measured on day 1. Column E: cells challenged with NALM6 on day 0 and day 2, killing measured on day 3. Column F: cells challenged with NALM6 on day 0, day 2 and day 4, killing measured on day 5. Column G: cells challenged with NALM6 on day 0, day 2, day 4 and day 6, killing measured on day 7. Column H: cells challenged with NALM6 on day 0, day 2, day 4, day 6 and day 8, killing measured on day 9.

Table 9. Enhanced Survival of Primary T-Cells

Gene transfer polynucleotides comprising piggyBac-like transposons were constructed as described in Section 6.2.1.5. Each transposon comprised one putative survival-enhancing gene, ESR gene or control gene. Eight samples of human primary T-cells from donor 1 (columns C-F) and eight samples of human primary T-cells from donor 2 (columns G-J) were prepared by co-transfection of 1 μg DNA of a single transposon and 100 ng mRNA encoding transposase with sequence given by SEQ ID NO: 37. The name of the gene is given in column A, and the SEQ ID NO of the gene is given in column B. Cells were cultured for 42 days, with samples taken at various times post-transfection for analysis by FACS of the presence of CD8 as a T-cell marker, and the expression of GFP as an indicator of the presence of the gene transfer polynucleotide in the genome of the T-cell. Columns C-J show the percentage of analyzed cells expressing CD8 on their surface (i.e. CD8+ T-cells) that were also expressing GFP, 1 day (columns C and G), 14 days (columns D and H), 28 days (columns E and I) and 42 days (columns F and J) post-transfection.

Table 10. Enhanced Activity of Primary T-Cells Expressing Bcl-XL

Gene transfer polynucleotides comprising piggyBac-like transposons were constructed and transfected into T-cells from 3 different donors as described in Section 6.2.1.6. After 240 days cells were challenged with a NALM6 B-cell tumor line, as described in Section 6.2.1.6. Column A shows the donor ID, column B shows whether the cells contained a transposon comprising a gene encoding Bcl-XL, column C shows whether the culture also contained a CD3/CD19-binding BiTE. Columns D-H show the % of NALM6 killing observed using a luminescence assay. Column D: cells challenged with NALM6 on day 0 and killing measured on day 1. Column E: cells challenged with NALM6 on day 0 and day 2, killing measured on day 3. Column F: cells challenged with NALM6 on day 0, day 2 and day 4, killing measured on day 5. Column G: cells challenged with NALM6 on day 0, day 2, day 4 and day 6, killing measured on day 7. Column H: cells challenged with NALM6 on day 0, day 2, day 4, day 6 and day 8, killing measured on day 9.

Table 11. Enhanced Activity of Primary T-Cells Expressing Survivin and CD28-D124E-T195P

Gene transfer polynucleotides comprising piggyBac-like transposons were constructed and transfected into T-cells as described in Section 6.2.1.6b. GFP-expressing CD8+ T-cells were sorted by FACS then challenged with a NALM6 B-cell tumor line, as described in Section 6.2.1.6b. Column A shows whether the cells were expressing a Survivin gene encoded on a heterologous polynucleotide, column B shows whether the cells were expressing a CD28-D124E-T195P gene encoded on a heterologous polynucleotide, column C shows whether the cells were expressing a Bcl-XL gene encoded on a heterologous polynucleotide. Columns D-I show the % of NALM6 killing observed using a luminescence assay. Column D: cells challenged with NALM6 on day 0 and killing measured on day 1. Column E: cells challenged with NALM6 on day 0 and day 2, killing measured on day 3. Column F: cells challenged with NALM6 on day 0, day 2 and day 4, killing measured on day 5. Column G: cells challenged with NALM6 on day 0, day 2, day 4 and day 6, killing measured on day 7. Column H: cells challenged with NALM6 on day 0, day 2, day 4, day 6 and day 8, killing measured on day 9. Column I: cells challenged with NALM6 on day 0, day 2, day 4, day 6, day 8 and day 10, killing measured on day 11.

Table 12. Proliferation of Primary T-Cells Stimulated by Anti-CD28/OX40 ESR

A gene transfer polynucleotide comprising an anti-CD28/OX40 ESR encoded on a piggyBac-like transposon was constructed as described in Section 6.2.2.1. A control transposon comprised the Herpes Simplex virus thymidine kinase (HSV-TK) gene instead of the ESR. Samples of human primary T-cells from two donors were prepared by co-transfection of 1 μg DNA of the transposon and 100 ng mRNA encoding transposase with sequence given by SEQ ID NO: 37. Cells were cultured for the number of days shown in column A before being analyzed by FACS for the presence of CD8 as a T-cell marker, and the expression of GFP as an indicator of the presence of the gene transfer polynucleotide in the genome of the T-cell. The percentage of CD8-expressing cells that also expressed GFP are shown in columns B-E: donor 1 cells transfected with anti-CD28/OX40 ESR (column B), donor 1 cells transfected with HSV-TK (column B), donor 2 cells transfected with anti-CD28/OX40 ESR (column C), donor 2 cells transfected with HSV-TK (column D). ND=not done.

Table 13. Proliferation of Primary T-Cells Stimulated by ESR FAS/4-1BB Plus Casp7-DN

A gene transfer polynucleotide encoding an ESR in which the extracellular domain of FAS was fused with the transmembrane and intracellular domains of 4-1BB was constructed on a piggyBac-like transposon as described in Section 6.2.2.2. A second gene transfer polynucleotide encoding a dominant negative inhibitor of apoptosis Casp7-DN was constructed on a second piggyBac-like transposon as described in Section 6.2.2.2. Samples of human primary T-cells from two donors were prepared by co-transfection of 0.5 μg DNA of each transposon and 100 ng mRNA encoding transposase with sequence given by SEQ ID NO: 37. Cells were cultured for the number of days shown in column A before being analyzed by FACS for the presence of CD8 as a T-cell marker, and the expression of GFP as an indicator of the presence of the gene transfer polynucleotide in the genome of the T-cell. Column B shows the percentage of CD8-expressing cells that also expressed GFP.

### TABLES

TABLE 1

| | A | B | C |
|---|---|---|---|
| | Day | Xenopus | Bombyx |
| 1 | 5 | 85 | 84 |
| 2 | 10 | 50 | 42 |
| 3 | 15 | 18 | 27 |
| 4 | 22 | 16 | 25 |
| 5 | 34 | 20 | 30 |
| 6 | 55 | 20 | 35 |

TABLE 2

| | A | B | C | D | E | F | G | H | I |
|---|---|---|---|---|---|---|---|---|---|
| | Promoter name | Promoter SEQ ID NO | Intron SEQ ID NO | day 2 | day 8 | day 16 | day 23 | % decline | HEK |
| 1 | EF1 (Rn) | 94 | 155 | 87 | 62 | 24 | 25 | 0.71 | 14,526 |
| 2 | EEF2 (Rn) | 108 | 157 | 54 | 30 | 32 | 30 | 0.44 | 7,475 |
| 3 | Ubb | 95 | 159 | 59 | 51 | 50 | 48 | 0.19 | 3,241 |
| 4 | GAPDH (Rn) | 98 | 156 | 51 | 48 | 51 | 54 | −0.06 | 4,068 |
| 5 | PGKc | 115 | N/A | 61 | 56 | 56 | 57 | 0.07 | 1,255 |
| 6 | EF1 (Hs) | 132 | 158 | 76 | 37 | 36 | 37 | 0.51 | 13,627 |

TABLE 3

| | A | B | C | D | E |
|---|---|---|---|---|---|
| | Promoter | Promoter | Intron | day 8 | day 8 |
| | name | SEQ ID NO | SEQ ID NO | MFI | CD19/cell |
| 1 | EF1 (Rn) | 94 | 157 | 33,766 | 78,294 |
| 2 | EEF2 (Rn) | 108 | 157 | 18,355 | 42,560 |
| 3 | Ubb | 95 | 159 | 39,927 | 92,579 |
| 4 | GAPDH (Rn) | 98 | 156 | 49,764 | 115,389 |
| 5 | PGKc | 115 | N/A | 38,829 | 90,034 |
| 6 | EF1 (Hs) | 132 | 158 | 48,174 | 111,702 |
| 7 | B cells | N/A | N/A | 9,488 | 22,000 |

TABLE 4

| | A | B | C | D |
|---|---|---|---|---|
| | Promoter | Promoter | day 11 | day 11 |
| | name | SEQ ID NO | MFI | CD19% |
| 1 | GAPDH (Hs) | 97 | 5,975 | 9.1 |
| 2 | GADPH (Rn) | 98 | 7,570 | 8.6 |
| 3 | EEF2(Rn) | 108 | 8,490 | 10.8 |
| 4 | EEF2(Mm) | 109 | 6,936 | 3.3 |
| 5 | D4SV40 | 110 | 2,531 | 2.0 |
| 6 | HSVTK-XPRT | 111 | 4,238 | 7.4 |
| 7 | HSVTK | 112 | 5,400 | 5.1 |
| 8 | MC1 | 113 | 10,528 | 6.4 |
| 9 | EEF2(Mm) + intron | 114 | 10,451 | 4.9 |

TABLE 5

| | | B | | D | E | | G | H |
|---|---|---|---|---|---|---|---|---|
| | A | GENE 1 | C | GENE 2 | lymphocyte | F | live | GFP + |
| | GENE 1 | SEQ ID NO | GENE 2 | SEQ ID NO | % | live % | CD8 (%) | CD8 (%) |
| 1 | CD28-D124E + T195P | 251 | none | N/A | 59.5 | 62.8 | 24.2 | 38.5 |
| 2 | VHH-CD3e Fusion | 319 | none | N/A | 54.1 | 69.8 | 8.4 | 12 |
| 3 | VHH-CD3d Fusion | 320 | none | N/A | 47 | 59.7 | 7.2 | 12 |
| 4 | Survivin | 237 | none | N/A | 58 | 75.2 | 7.4 | 9.8 |
| 5 | Survivin | 237 | none | N/A | 50.2 | 67.9 | 6.5 | 9.7 |
| 6 | ESR: Anti-CD28-CD28 T195P | 318 | none | N/A | 54.3 | 71.2 | 5.2 | 7.3 |
| 7 | ESR: Anti-CD28-CD28 T195P | 318 | none | N/A | 49.1 | 67.5 | 4.8 | 7.1 |
| 8 | ESR: TNFR1-TM(TNFR1)-41BB | 302 | none | N/A | 50.4 | 74.7 | 4.5 | 6 |
| 9 | ESR: TNFR1-TM(TNFR1)-41BB | 302 | none | N/A | 60.9 | 14.8 | 0.64 | 4.4 |
| 10 | CD19 | 228 | none | N/A | 55.4 | 75.5 | 2.3 | 3.1 |
| 11 | Survivin | 237 | none | N/A | 28.9 | 72.9 | 2.1 | 3 |
| 12 | Tisagenlecleucel | 229 | none | N/A | 47.9 | 63.7 | 1.8 | 2.9 |
| 13 | Tisagenlecleucel | 229 | none | N/A | 49.3 | 62.1 | 1.2 | 1.9 |
| 14 | Bcl-X1 | 238 | none | N/A | 37.6 | 88.6 | 0.5 | 0.6 |
| 15 | VHH-CD3d Fusion | 320 | none | N/A | 51.1 | 75.5 | 0.3 | 0.4 |
| 16 | Fas/4-1BB | 319 | Casp7-DN | 262 | 14.4 | 35.1 | 17.9 | 51 |
| 17 | STAT3-Y640F | 246 | PIK3CA-L1001P | 257 | 21.3 | 64.8 | 30 | 46.3 |
| 18 | PTEN Antagonist: PAP1 | 258 | PIK3CA-L1001P | 257 | 30.3 | 46.3 | 7.4 | 16 |
| 19 | Bcl2 | 270 | v-src | 260 | 58.9 | 73.4 | 8.3 | 11.3 |
| 20 | CD28-D124E + T195P | 251 | PIK3CA-L1001P | 257 | 46.2 | 79.5 | 1.6 | 2 |
| 21 | Fas/4-1BB | 274 | PIK3CA-L1001P | 257 | 58.4 | 75.9 | 1.3 | 1.7 |
| 22 | RHOA-G17V | 252 | PIK3CA-L1001P | 257 | 39.8 | 51.6 | 0.5 | 0.9 |
| 23 | PTEN Antagonist: PAP4 | 259 | PIK3CA-L1001P | 257 | 46 | 64.9 | 0.3 | 0.5 |

TABLE 6

| | | | B | C |
|---|---|---|---|---|
| | | A | Receptor + | Receptor + |
| | Sample | Receptor | Survivin | CD28 |
| 1 | SEQ ID NO | 225 | 226 | 227 |
| 2 | GFP (%) at seeding | 7.1 | 10.8 | 8.2 |
| 3 | CD19+ at Day 3 | 89.2 | 29.1 | 23.1 |
| 4 | CD8+ at Day 3 | 6.3 | 42.7 | 50 |
| 5 | CD19+ Day 7 | 86.3 | 1.7 | 0.2 |
| 6 | CD8+ at Day 7 | 2.3 | 89.4 | 92.1 |

TABLE 7

| | A | B | C |
|---|---|---|---|
| | T-cell | Transposon | Survival |
| | transposon genes | SEQ ID NO. | length (days) |
| 1 | N/A | N/A | 24 |
| 2 | N/A | N/A | 25 |
| 3 | Receptor | 225 | 30 |
| 4 | Receptor + Survivin | 226 | 34 |
| 5 | Receptor + CD28 | 227 | 34 |

TABLE 8

| | | A | B | C | | | | | |
|---|---|---|---|---|---|---|---|---|---|
| | | ex vivo time (months) | survivin | CD28 mutant | D | E | F | G | H |
| 1 | Number of challenges | | | | 1 | 2 | 3 | 4 | 5 |
| 2 | Car-T + NALM6 | 10 | no | no | 100% | 85% | 47% | 23% | 10% |
| 3 | Car_Suv-T + NALM6 | 10 | yes | no | 100% | 99% | 82% | 80% | 76% |
| 4 | Car_CD28mut-T + NALM6 | 10 | no | yes | 100% | 100% | 84% | 85% | 82% |
| 5 | CAR02R Car-T + NALM6 | 4 | no | no | 100% | 100% | 51% | 28% | 27% |
| 6 | CAR02R Car_Suv-T + NALM6 | 4 | yes | no | 100% | 100% | 96% | 87% | 90% |
| 7 | CAR02R Car_CD28mut-T + NALM6 | 4 | no | yes | 100% | 100% | 97% | 87% | 91% |

TABLE 9

| | | | | C | D | E | F | G | H | I | J |
|---|---|---|---|---|---|---|---|---|---|---|---|
| | | A | B | donor 1 | donor 1 | donor 1 | donor 1 | donor 2 | donor 2 | donor 2 | donor 2 |
| | | Gene Name | Gene SEQ ID NO | 1 day | 14 days | 28 days | 42 days | 1 day | 14 days | 28 days | 42 days |
| 1 | 342140 | STAT3_D661Y | 247 | 6 | 4.7 | 14 | 61.5 | 5.4 | 10.1 | 8.6 | 31.2 |
| 2 | 335791 | Bcl-XI | 238 | 17.5 | 24.5 | 31.4 | 60.7 | 20.6 | 11.6 | 39 | 42 |
| 3 | 342141 | STAT3_S614R_Y640F | 248 | 5.6 | 3.9 | 11.5 | 54.5 | 17.8 | 6.2 | 6.9 | 36.7 |
| 4 | 340909 | ESR: TNFR1-TM-CD27 | 301 | 30.6 | 34.1 | 8.9 | 49.5 | 32.5 | 8.1 | 4.7 | 5.2 |
| 5 | 340911 | ESR: TNFR1-TM-41BB | 302 | 9.6 | 8 | 17 | 33.4 | 8.4 | 10.2 | 7.5 | 6.8 |
| 6 | 335845 | PLCG1 (S345F) | 254 | 7.2 | 5.8 | 5.6 | 25.6 | 11.8 | 5.5 | 33.8 | 2.8 |
| 7 | 335829 | HSV-TK | 231 | 14.3 | 1.4 | 2.4 | 0.6 | 15.3 | 1.7 | 4.6 | 0.8 |
| 8 | 335829 | HSV-TK | 231 | 7.5 | 1.3 | 1.3 | 3.8 | 8 | 4.2 | 2.3 | 0.6 |

TABLE 10

| | | A | B | C | | | | | |
|---|---|---|---|---|---|---|---|---|---|
| | | Donor | Bcl-XL | BiTE | D | E | F | G | H |
| 1 | Number of challenges | | | | 1 | 2 | 3 | 4 | 5 |
| 2 | ND82 Bcl-Xl-T + N6 | 82 | yes | no | 21% | 9% | 24% | 23% | 22% |
| 3 | ND84 Bcl-Xl-T + N6 | 84 | yes | no | 24% | 10% | 27% | 28% | 29% |
| 4 | ND81 Bcl-Xl-T + N6 | 81 | yes | no | 24% | 8% | 24% | 22% | 21% |
| 5 | T + N6 | 81 | no | no | 24% | 23% | 32% | 20% | 39% |
| 6 | ND81 Bcl-Xl-T + N6 + BiTE | 81 | yes | yes | 87% | 98% | 95% | 96% | 95% |
| 7 | ND82 Bcl-Xl-T + N6 + BiTE | 82 | yes | yes | 97% | 98% | 95% | 94% | 94% |
| 8 | ND84 Bcl-Xl-T + N6 + BiTE | 84 | yes | yes | 99% | 98% | 97% | 97% | 95% |
| 9 | T + N6 + BiTE | 81 | no | yes | 88% | 97% | 72% | 62% | 59% |

TABLE 11

| | | A | B | C | | | | | | |
|---|---|---|---|---|---|---|---|---|---|---|
| | | survivin | CD28 mutant | Bcl-XL | D | E | F | G | H | I |
| 1 | Number of challenges | | | | 1 | 2 | 3 | 4 | 5 | 6 |
| 2 | Car-T + NALM6 | no | no | no | 70% | 97% | 69% | 59% | 58% | 53% |
| 3 | Car_Suv-T + NALM6 | yes | no | no | 69% | 98% | 86% | 87% | 84% | 85% |
| | Car_CD28mut- | | | | | | | | | |
| 4 | T + NALM6 | no | yes | no | 68% | 98% | 88% | 87% | 85% | 85% |
| | CAR_Bcl-xL- | | | | | | | | | |
| 5 | T + NALM6 | no | no | yes | 70% | 100% | 99% | 96% | 94% | 94% |
| 6 | Mock-T + NALM6 | no | no | no | 12% | 15% | 15% | 16% | 15% | 13% |

TABLE 12

| | B | C | D | E |
|---|---|---|---|---|
| | Donor 1: | Donor 1: | Donor 2: | Cdonor 2: |
| | ESR | Control | ESR | Control |
| A | CD8+ | CD8+ | CD8+ | CD8+ |
| Days | GFP+ (%) | GFP+ (%) | GFP+ (%) | GFP+ (%) |
| 1 | 18.4 | 14.3 | ND | 15.3 |
| 14 | 94.3 | 1.4 | 13.5 | 1.7 |
| 28 | ND | 2.4 | 98.4 | 4.6 |

TABLE 13

| | CD8+ |
|---|---|
| Day | GFP+ (%) |
| 1 | 4.5 |
| 7 | 1.9 |
| 28 | 31 |
| 48 | 50.4 |
| 54 | 97.6 |

The invention includes the follow numbered embodiments:
1. A polynucleotide comprising an immune cell survival-enhancing gene comprising a nucleic acid encoding a protein operably linked to a heterologous regulatory sequence effective for expression of the protein within an immune cell thereby enhancing survival of the immune cell.
2. The polynucleotide of embodiment 1, wherein the immune cell survival-enhancing gene encodes a naturally occurring protein comprising an activating mutation.
3. The polynucleotide of embodiment 1 or 2, wherein the immune cell survival-enhancing gene encodes a protein selected from STAT3, CD28, RhoA, PLCG, STAT5B or CCND1 comprising an activating mutation.
4. The polynucleotide of embodiment 3, wherein the immune cell survival-enhancing gene encodes STAT3, wherein the STAT3 comprises one or more of the following activating mutations: F174S, H410R, S614R, E616K, G618R, Y640F, N6471, E652K, K658Y, K658R, K658N, K658M, K658R, K658H, K658N, D661Y or D661V.
5. The polynucleotide of embodiment 3 wherein the immune cell survival-enhancing gene encodes CD28, wherein the CD28 comprises one or more of the following activating mutations: D124E, D124V, T1951 or T195P.
6. The polynucleotide of embodiment 3, wherein the immune cell survival-enhancing gene encodes RhoA, wherein the RhoA comprises one or more of the following activating mutations: G17V or K18N.
7. The polynucleotide of embodiment 3, wherein the immune cell survival-enhancing gene encodes PLCG, wherein the PLGC comprises one of the following activating mutations: S345F, S520F or R707Q.
8. The polynucleotide of embodiment 3, wherein the immune cell survival-enhancing gene encodes STAT5B, wherein the STAT5B comprises one or more of the following activating mutations: N642H, T648S, S652Y, Y665F or P267A.
9. The polynucleotide of embodiment 3, wherein the immune cell survival-enhancing gene encodes CCND1, wherein the CCND1 comprises one or more of the following activating mutations: E36G, E36Q, E36K, A39S, S41L, S41P, S41T, V42E, V42A, V42L, V42M, Y44S, Y44D, Y44C, Y44H, K46T, K46R, K46N, K46E, C47G, C47R, C47S, C47W, P199R, P199S, P199L, S201F, T2851, T285A, P286L, P286H, P286S, P286T or P286A.
10. The polynucleotide of embodiment 1, wherein the immune cell survival-enhancing gene encodes a naturally occurring human protein.
11. The polynucleotide of embodiment 10, wherein the immune cell survival-enhancing gene encodes a protein selected from Survivin, Bcl2, Bcl6 or Bcl-XL.
12. The polynucleotide of embodiment 1, wherein the immune cell survival-enhancing gene encodes an inhibitor of apoptosis.
13. The polynucleotide of any preceding embodiment, wherein the heterologous promoter is selected from an EF1 promoter, a PGK promoter, a GAPDH promoter, an EEF2 promoter, a ubiquitin promoter, an SV40 promoter or an HSVTK promoter
14. The polynucleotide of any one of embodiments 1-12, wherein the heterologous promoter is selected from SEQ ID Nos: 94-154.
15. The polynucleotide of any preceding embodiment, wherein the polynucleotide further comprises a pair of sequences selected from SEQ ID NOs: 6 and 7, or SEQ ID NOs: 14 and 15, or SEQ ID NOs: 18 and 19, or SEQ ID NOs: 20 and 21, or SEQ ID NOs: 26 and 27, or SEQ ID NOs: 399 and 400.
16. The polynucleotide of any preceding embodiment, wherein the half-life of an immune cell whose genome comprises the polynucleotide is increased by at least 25% relative to the half-life of an immune cell whose genome does not comprise the polynucleotide.
17. The polynucleotide of any preceding embodiment, wherein the maximum life span of an immune cell whose genome comprises the polynucleotide is increased by at least 25% relative to the maximum life span of an immune cell whose genome does not comprise the polynucleotide.
18. The polynucleotide of any preceding embodiment, wherein the doubling time of an immune cell whose genome does not comprise the polynucleotide is greater by at least 25% relative to the doubling time of an immune cell whose genome comprises does comprise the polynucleotide.
19. The polynucleotide of any preceding embodiment, wherein the proliferation rate of an immune cell whose genome comprises the polynucleotide is increased by at least 25% relative to the proliferation rate of an immune cell whose genome does not comprise the polynucleotide.
20. The polynucleotide of any preceding embodiment, wherein the survival upon repeated antigen challenge of a T-cell whose genome comprises the polynucleotide is increased by at least 25% relative to the survival of an immune cell whose genome does not comprise the polynucleotide.
21. A transposon comprising the polynucleotide of any preceding embodiment.
22. A lentiviral vector comprising the polynucleotide of any one of embodiments 1-20.
23. A method for creating a modified immune cell, the method comprising introducing into an immune cell a polynucleotide encoding an inhibitor of apoptosis operably linked to a heterologous promoter.
24. The method of embodiment 23, wherein the polynucleotide further comprises transposon ends, and wherein the method further comprises introducing into the immune cell a corresponding transposase, such that the polynucleotide encoding the inhibitor of apoptosis is transposed into the genome of the immune cell.
25. The method of embodiment 23 or 24, wherein the transposase is introduced as a nucleic acid encoding the transposase.
26. The method of embodiment 25, wherein the nucleic acid is an mRNA.
27. The method of embodiment 24 or 25, wherein the nucleic acid encoding the transposase is operably linked to a promoter that is active in the immune cell.
28. The method of embodiment 24, wherein the transposon and transposase are introduced into the immune cell at the same time.
29. The method of embodiment 24, wherein the transposon and transposase are introduced into the immune cell at different times.
30. The method of any one of embodiments 23-29, wherein the immune cell is a T-cell, the method further comprising introducing into the immune cell a gene encoding a receptor capable of binding to an antigen, wherein binding of the receptor to a target cell which displays the antigen on its surface causes the T-cell to kill the target cell.
31. The method of any one of embodiments 23-29, wherein the inhibitor of apoptosis is selected from Survivin, Bcl2, Bcl6, Bcl-XL or a dominant negative mutant of Casp3, Casp7, Casp8, Casp9 or Casp10.
32. A method for creating a modified immune cell, the method comprising introducing into an immune cell a polynucleotide encoding a protein selected from STAT3, CD28, RhoA, PLCG, STAT5B or CCND1, wherein the protein comprises an activating mutation operably linked to a heterologous promoter.
33. A method for creating a modified immune cell, the method comprising introducing into an immune cell a polynucleotide encoding a polypeptide comprising
   a. a sequence derived from the extracellular domain of a receptor that normally transmits an inhibitory signal to an immune cell
   b. a sequence derived from the intracellular domain of an intracellular domain of a receptor that transmits a stimulatory signal to an immune cell
   c. a transmembrane domain and wherein the polypeptide does not comprise a CD3 zeta intracellular domain.
34. The method of embodiment 33, wherein the extracellular domain comprises a sequence selected from SEQ ID NOs: 322-340.
35. The method of embodiment 33 or 34, wherein the intracellular domain comprises a sequence selected from SEQ ID NOs: 341-364.
36. The method of embodiment 33, wherein the polypeptide comprises a sequence selected from SEQ ID NOs: 274-318.
37. An immune cell whose genome comprises the polynucleotide of any one of embodiments 1-22.
38. The immune cell of embodiment 37, wherein the half-life of the immune cell is increased by at least 25% relative to the half-life of an immune cell whose genome does not comprise the polynucleotide of embodiment 1.
39. The immune cell of embodiment 37 or 38, wherein the maximum life span of the immune cell is increased by at least 25% relative to the maximum life span of an immune cell whose genome does not comprise the polynucleotide of embodiment 1.
40. The immune cell of any one of embodiments 37-39, wherein the doubling time of an immune cell whose genome does not comprise the polynucleotide of embodiment 1 is increased by at least 25% relative to the half-life of an immune cell whose genome comprises the polynucleotide of embodiment 1.
41. The immune cell of any one of embodiments 37-40, wherein the proliferation rate of the immune cell is increased by at least 25% relative to the proliferation rate of an immune cell whose genome does not comprise the polynucleotide of embodiment 1.
42. The immune cell of any one of embodiments 37-41, wherein the survival upon repeated antigen challenge of a T-cell whose genome comprises the polynucleotide is increased by at least 25% relative to the survival of an immune cell whose genome does not comprise the polynucleotide.
43. The immune cell of any one of embodiments 37-42, wherein the immune cell is a T-cell.
44. The immune cell of any one of embodiments 37-42, wherein the immune cell is a B-cell.
45. The immune cell of any one of embodiments 37-42, wherein the immune cell is a

$$human cell .$$

46. The immune cell of any one of embodiments 37-42, wherein the immune cell is a primate cell, a rodent cell, a cat cell, a dog cell or a horse cell.
47. The polynucleotide of embodiment 1, wherein the immune cell survival-enhancing gene encodes an enhanced signaling receptor (ESR) wherein the ESR comprises
   a. a sequence derived from the extracellular domain of a receptor that normally transmits an inhibitory signal to an immune cell
   b. a sequence derived from the intracellular domain of an intracellular domain of a receptor that transmits a stimulatory signal to an immune cell
   c. a transmembrane domain
   and wherein the ESR does not comprise a CD3 zeta intracellular domain
48. The polynucleotide of embodiment 47, wherein the extracellular domain (a) is from a human protein selected from TNFRSF3 (LTRβ), TNFRSF6 (Fas), TNFRSF8 (CD30), TNFRSF10A (DR4), TNFRSF10B (DR5), TNFRSF19 (TROY), TNFRSF21 (DR6) and CTLA4.
49. The polynucleotide of embodiment 47, wherein the ESR comprises a sequence that is at least 90% identical to a sequence selected from SEQ ID NO: 322-340.
50. The polynucleotide of any one of embodiments 47-49, wherein the intracellular domain (b) is from a human protein selected from TNFRSF4 (OX40), TNFRSF5 (CD40), TNFRSF7 (CD27), TNFRSF9 (4-1BB), TNFRSF11A (RANK), TNFRSF13B (TACI), TNFRSF13C (BAFF-R), TNFRSF14 (HVEM), TNFRSF17 (CD269), TNFRSF18 (GITR), CD28, CD28H (TMIGD2), Inducible T-cell Costimulator (ICOS/CD278), DNAX Accessory Molecule-1 (DNAM-1/CD226), Signaling Lymphocytic Activation Molecule (SLAM/CD150), T-cell Immunoglobulin and Mucin domain (TIM-1/HAVcr-1), interferon receptor alpha chain (IFNAR1), interferon receptor beta chain IFNAR2), interleukin-2 receptor beta subunit (IL2RB), interleukin-2 receptor gamma subunit (IL2RG), Tumor Necrosis Factor Superfamily 14 (TNFSF14/LIGHT), Natural Killer Group 2 member D (NKG2D/CD314) and CD40 ligand (CD40L)
51. The polynucleotide of any one of embodiments 47-50, wherein the ESR comprises a polypeptide whose sequence is at least 90% identical to a sequence selected from SEQ ID NOs: 341-364.
52. The polynucleotide of any one of embodiments 47-51, wherein the ESR comprises a polypeptide whose sequence is at least 90% identical to a sequence selected from SEQ ID NOs: 365-396.
53. The polynucleotide of any one of embodiments 45-52, wherein the ESR comprises a polypeptide whose sequence is at least 90% identical to a sequence selected from SEQ ID NOs: 274-318.
54. The polynucleotide of any one of embodiments 47-53, wherein the polynucleotide further comprises a segment encoding an inhibitor of apoptosis operably linked to a heterologous promoter.
55. An immune cell whose genome comprises the polynucleotide of any one of embodiments 47-54.
56. The immune cell of embodiment 55, wherein the immune cell genome further comprises a segment encoding an inhibitor of apoptosis operably linked to a heterologous promoter.
57. A method for creating a modified immune cell, the method comprising
   a. introducing into the immune cell the polynucleotide of embodiment 47.
   b. introducing into the immune cell a polynucleotide encoding an inhibitor of apoptosis, operably linked to a heterologous promoter.
58. The method of embodiment 57, wherein the two polynucleotides are introduced into the immune cell at the same time.
59. A method of identifying a protein enhancing survival of an immune cell, comprising sequencing nucleic acids encoding proteins from a cancerous immune cell to identify a nucleic acid encoding a protein with a mutation;
   transforming an immune cell with the nucleic acid encoding the protein with the mutation; and determining whether the immune cell has enhanced survival.

### REFERENCES

All references cited herein are incorporated herein by reference in their entirety and for all purposes to the same extent as if each individual publication or patent or patent application was specifically and individually indicated to be incorporated by reference in its entirety for all purposes. If different content is associated with a citation at different times, the content associated with the citation at the priority date of the invention is meant.

Many modifications and variations of this invention can be made without departing from its spirit and scope, as will be apparent to those skilled in the art. The specific embodiments described herein are offered by way of example only, and the invention is to be limited only by the terms of the appended claims, along with the full scope of equivalents to which such claims are entitled.

1.-59. (canceled)

60. A polynucleotide, comprising:
a first nucleotide sequence encoding a chimeric antigen receptor comprising an extracellular domain that specifically binds to a CD19 antigen; and
a second nucleotide sequence encoding either an apoptosis inhibitor selected from the group consisting of SEQ ID NOs: 237, 238, and 271 or an activating mutant consisting of SEQ ID NO. 251.

61. The polynucleotide of claim 60, wherein the second nucleotide sequence encodes an apoptosis inhibitor consisting of SEQ ID NO: 237.

62. The polynucleotide of claim 60, wherein the second nucleotide sequence encodes an apoptosis inhibitor consisting of SEQ ID NO: 238.

63. The polynucleotide of claim 60, wherein the second nucleotide sequence encodes an activating mutant consisting of SEQ ID NO. 251.

64. The polynucleotide of claim 60, further comprising a transposon.

65. The polynucleotide of claim 64, wherein:
(A) the second nucleotide sequence is operably linked to a heterologous promoter; and
(B) the polynucleotide is flanked by a pair of transposon ends.

66. The polynucleotide of claim 60, further comprising a lentiviral vector.

67. A T cell expressing a polynucleotide, the polynucleotide comprising:
a first nucleotide sequence encoding a chimeric antigen receptor comprising an extracellular domain that specifically binds to a CD19 antigen; and
a second nucleotide sequence encoding either an apoptosis inhibitor selected from the group consisting of SEQ ID NOs: 237, 238, and 271 or an activating mutant consisting of SEQ ID NO. 251.

68. The T cell of claim 67, wherein the second nucleotide sequence encodes an apoptosis inhibitor consisting of SEQ ID NO: 237.

69. The T cell of claim 67, wherein the second nucleotide sequence encodes an apoptosis inhibitor consisting of SEQ ID NO: 238.

70. The T cell of claim 67, wherein the second nucleotide sequence encodes an activating mutant consisting of SEQ ID NO. 251.

71. The T cell of claim 67, wherein:
a persistence of the T cell is increased relative to a persistence of a T cell that does not comprise the first and second nucleotide sequences; and/or
a proliferation rate of the T cell is increased relative to a proliferation rate of a T cell that does not comprise the first and second nucleotide sequences.

72. The T cell of claim 67, wherein the polynucleotide is integrated into the genome of the T cell.

73. The T cell of claim 67 for use in the treatment of cancer.

74. The T cell of claim 67 for use in the treatment of a B cell malignancy.

75. A kit, comprising:
(A) a polynucleotide, comprising:
(1) a first nucleotide sequence encoding a chimeric antigen receptor comprising an extracellular domain that specifically binds to a CD19 antigen;
(2) a second nucleotide sequence encoding either an apoptosis inhibitor selected from the group consisting of SEQ ID NOs: 237, 238, and 271 or an activating mutant consisting of SEQ ID NO. 251; and
(3) a transposon; and
(B) a transposase capable of transposing the transposon or a nucleic acid encoding a transposase capable of transposing the transposon.

76. The kit of claim 75, wherein the second nucleotide sequence encodes an apoptosis inhibitor consisting of SEQ ID NO: 237.

77. The kit of claim 75, wherein the second nucleotide sequence encodes an apoptosis inhibitor consisting of SEQ ID NO: 238.

78. The kit of claim 75, wherein the second nucleotide sequence encodes an activating mutant consisting of SEQ ID NO. 251.

79. The kit of claim 75, further comprising an anti-hCD19-CD3 bispecific T cell engager.

80. The kit of claim 75, wherein, when the kit comprises a nucleic acid encoding the transposase, the nucleic acid encoding the transposase is an mRNA.
